(19) United States
(12) Patent Application Publication
(10) Pub. No.: US 2026/0363547 A1
(43) Pub. Date: Nov. 19, 2026

(54) SYSTEMS AND METHODS FOR ANALYZING THE EYE

(71) Applicant: Lightfield Medical Inc., Minneapolis, MN (US)

(72) Inventors: John Stephen Berestka, Minneapolis, MN (US); Jan Bonel, Gava (ES); Dimitrios Christaras, Athens (GR); Alexandros Pennos, Larisa (GR); Harilaos Ginis, Athens (GR)

(21) Appl. No.: 19/725,714

(22) Filed: Jun. 30, 2026

Related U.S. Application Data
(63) Continuation of application No. PCT/US2026/024571, filed on Apr. 21, 2026.
(60) Provisional application No. 63/792,598, filed on Apr. 22, 2025.
(60) Provisional application No. 63/816,666, filed on Jun. 3, 2025.
(60) Provisional application No. 63/898,798, filed on Oct. 14, 2025.

(51) Int. Cl. A61B 3/14; A61B 3/00

(52) U.S. Cl. CPC A61B 3/14; A61B 3/0041; A61B 3/0075

(57) ABSTRACT

Medical devices and methods of use are disclosed to exam an eye of a patient. A medical device may include a digital projector as part of an illumination system. A medical device may include one or more cameras as part of an observation system to capture reflected light from the eye. The observation system may include one or more adjustable optical systems. The digital projector may produce a slit of light to be projected onto the eye. The slit of light and the observation system may be focused at a plurality of focal depths within the eye. The slit of light may be a compound slit.

### RELATED APPLICATIONS

This application is a continuation of PCT Application No. PCT/US2026/024571, filed Apr. 21, 2026, titled SYSTEMS AND METHODS FOR ANALYZING THE EYE which claims the benefit of U.S. Provisional Application No. 63/898,798, filed Oct. 14, 2025, titled SYSTEMS AND METHODS FOR ANALYZING THE EYE; U.S. Provisional Application No. 63/816,666, filed Jun. 3, 2025, titled SYSTEMS AND METHODS FOR ANALYZING THE EYE; and U.S. Provisional Application No. 63/792,598, filed Apr. 22, 2025, titled SYSTEMS AND METHODS FOR ANALYZING THE EYE, the entire disclosures of which are expressly incorporated by reference herein. The present application is further related to U.S. Provisional Application No. 63/547,628, filed Nov. 7, 2023, titled SYSTEMS AND METHODS FOR ANALYZING THE EYE; and PCT Application No. PCT/US2024/054811, filed Nov. 6, 2024, titled SYSTEMS AND METHODS FOR ANALYZING THE EYE, the entire disclosures of which are expressly incorporated by reference herein.

### TECHNICAL FIELD

The present invention relates to one or more imaging systems including an illumination system and a detection system.

### BACKGROUND

Ophthalmologists use a variety of devices for imaging of a patient's eye, including slit-lamps, ophthalmoscopes, fundus cameras, and scanning laser ophthalmoscopes (SLOs). The slit lamp is a versatile instrument used by ophthalmologists for examining a patient's eye. It consists of a microscope, an illumination source, and a mechanical support system to facilitate positioning the illumination source at various angles with respect to the eye. Ophthalmologists and optometrists typically examine the eye by first horizontally scanning across the eye using various slit beam thicknesses and orientations to examine the most anterior structures such as the cornea and conjunctiva. Then the examiner will adjust the focus plane posterior to horizontally scan across the anterior chamber of the eye. The focus is then adjusted more posteriorly to horizontally scan across the iris and anterior crystalline lens. The process is repeated again to examine the posterior aspect of the crystalline lens and anterior vitreous.

FIGS. 1 and 1A show a schematic view of a patient's eye. As shown in FIGS. 1 and 1A, the basic components of the eye 10 include a cornea 12, conjunctiva 14, an iris 16, a pupil 18, a crystalline lens 20, and a retina 22. An anterior chamber 24 is provided behind the cornea 12. A posterior chamber 40 is provided posterior of anterior chamber 24. The posterior chamber 40 includes the lens 20 which is positioned by the suspensory ligaments 34 of the eye. An anterior capsule 31 lines the anterior portion of the crystalline lens 20 and the posterior capsule 30 lines the posterior portion of the crystalline lens 20. The posterior capsule 30 and the suspensory ligaments 34 form the anterior boundary of the vitreous cavity 32. Light enters the front of the eye through the cornea 12, passes through the pupillary aperture 18 and then the lens 20. The curvatures of the cornea and lens invert and focus the light which is then projected onto the retina 22 at the back of the eye. The iris 16 functions as an “aperture” that opens and closes to regulate the amount of light entering the eye. The cornea, iris, pupil and lens are often referred to as the anterior segment of the eye. The retina 22 is a multi-layered structure that converts received light into a neural signal through a process known as “signal transduction.” The photoreceptors on the retina are known as rods and cones. These generate neural signals that are communicated to the brain by ganglion cells that form the optic nerve 24.

### SUMMARY

In an exemplary embodiment of the present disclosure, a method of examining an eye of a patient may be provided. The method may comprises the steps of: positioning an illumination system and an observation system of an examination system in a first spatial configuration relative to the eye; determining that a scan definition defining a plurality of images to be captured by at least one camera of the observation system may be executed with the illumination system and the observation system in the first spatial configuration, the plurality of images including a first image at a first focal plane of the eye and a second image at a second focal plane of the eye; and while maintaining the illumination system and the observation system in the first spatial configuration performing the following steps: (a) projecting a first pattern from the illumination system towards the eye, (b) receiving light reflected by the eye from the projecting step (a) with a first camera of the observation system to capture the first image of the plurality of images, the illumination system and the observation system both focused at the first focal plane relative to the eye during steps (a) and (b); (c) projecting a second pattern from the illumination system towards the eye, (d) receiving light reflected by the eye from the projecting step (c) with the first camera of the observation system to capture the second image of the plurality of images, the illumination system and the observation system both focused at the second focal plane relative to the eye during steps (c) and (d).

In an example thereof, the method may further comprise tracking a position of the eye; determining a first movement of the eye prior to step (c); and adjusting at least one characteristic of the illumination system based on the determined movement of the eye. In a variation thereof, the step of adjusting the at least one characteristic of the illumination system based on the determined first movement of the eye may include adjusting a focal plane of the illumination system. In another variation thereof, the step of adjusting the at least one characteristic of the illumination system based on the determined first movement of the eye may include adjusting a feature of the second pattern. In a further variation thereof, the feature of the second pattern may be a line of an image produced with a digital projector of the illumination system. In yet a further variation thereof, an offset of the line from an edge of the image may be adjusted.

In another example thereof, the method may further comprise tracking a position of the eye; determining a first movement of the eye prior to step (c); and adjusting at least one characteristic of the illumination system based on the determined movement of the eye, the step of adjusting the at least one characteristic of the illumination system based on the determined first movement of the eye includes adjusting an adjustable optical assembly of the illumination system. In a variation thereof, the step of adjusting the adjustable optical assembly of the illumination system may include the step of adjusting a focal length of a tunable lens. In another variation thereof, the step of adjusting the adjustable optical assembly of the illumination system may include the step of adjusting a position of a movable lens.

In yet another example thereof, the method may further comprise tracking a position of the eye; determining a first movement of the eye prior to step (c); adjusting at least one characteristic of the illumination system based on the determined movement of the eye; and adjusting at least one characteristic of the observation system based on the determined first movement of the eye. In a variation thereof, the step of adjusting the at least one characteristic of the observation system based on the determined movement of the eye may include adjusting a focal plane of the observation system. In another variation thereof, the step of adjusting the at least one characteristic of the observation system based on the determined first movement of the eye may include adjusting an adjustable optical assembly of the observation system. In a further variation thereof, the step of adjusting the adjustable optical assembly of the observation system may include the step of adjusting a focal length of a tunable lens. In still another variation thereof, the step of adjusting the adjustable optical assembly of the observation system may include the step of adjusting a position of a movable lens.

In still another example thereof, the method may further comprise the steps of determining a second movement of the eye causes at least a portion of the scan definition to be outside of a scan envelope of the examination system with the illumination system and the observation system in the first spatial configuration and adjusting a position of the illumination system and a position of the observation system of the examination system to a second spatial configuration relative to the eye whereat the portion of the scan definition may be within the scan envelope of the examination system, the second spatial configuration being different than the first spatial configuration. In a variation thereof, the step of adjusting the position of the illumination system and the observation system of the examination system to the second spatial configuration relative to the eye may include the step of adjusting a position of a movable base of the examination system supporting the illumination system and the observation system relative to a patient support of the examination system, the patient support being adapted to support the eye of the patient. In a further variation thereof, the step of adjusting the focal plane of the observation system may include analyzing at least one image captured by a camera with an image sensor tilted relative to a focal plane of the observation system. In still another variation thereof, the step of adjusting the focal plane of the observation system may include analyzing a plurality of images captured by a camera of the observation system, each image being at a respective focal depth and further comprising the step of analyzing a characteristic of each image to determine a position of the examination system relative to the eye.

In another exemplary embodiment of the present disclosure, a medical device for imaging at least a portion of an eye of a patient may be provided. The medical device may comprise an illumination system including a digital projector producing at least one test pattern image to illuminate the eye and a first adjustable optical assembly to project the at least one test pattern image towards the eye and an observation system including a second adjustable optical assembly positioned to receive imaging rays produced by reflection of light from the eye of the patient due to the projection of the at least one test pattern image towards the eye by the illumination system and a first camera configured receive the imaging rays from the second adjustable optical assembly. The illumination system and the observation system may be positioned in a first spatial configuration relative to the eye. The medical device may further comprise a controller operatively coupled to the illumination system and to the observation system, the controller may be configured to adjust the first adjustable optical assembly of the illumination system and the second adjustable optical system of the observation system to a plurality of focal depths relative to the eye while the illumination system and observation system are in the first spatial configuration. The controller may further be configured to (a) determine that a scan definition defining a plurality of images to be captured by at least one camera of the observation system may be executed with the illumination system and the observation system in the first spatial configuration, the plurality of images including a first image at a first focal plane and a second image at a second focal plane, the second focal plane being offset from the first focal plane; and while maintaining the illumination system and the observation system in the first spatial configuration the controller may be further configured to perform the following steps: (b) projecting a first pattern from the illumination system towards the eye with a first test pattern image of the at least one test pattern image produced by the digital projector, (c) receiving light reflected by the eye from the projecting step (b) with the first camera of the observation system to capture the first image of the plurality of images, the illumination system and the observation system both focused at the first focal plane relative to the eye during steps (b) and (c); (d) projecting a second pattern from the illumination system towards the eye with a second test pattern image of the at least one test pattern image produced by the digital projector, and (e) receiving light reflected by the eye from the projecting step (d) with the first camera of the observation system to capture the second image of the plurality of images, the illumination system and the observation system both focused at the second focal plane relative to the eye during steps (d) and (e).

In an example thereof, the controller may be further configured to perform the following steps: tracking a position of the eye; determining a first movement of the eye prior to step (d); and adjusting at least one characteristic of the illumination system based on the determined movement of the eye. In a variation thereof, the adjusting of the at least one characteristic of the illumination system may be based on the determined first movement of the eye includes adjusting a focal plane of the illumination system. In another variation thereof, adjusting the at least one characteristic of the illumination system based on the determined first movement of the eye may include adjusting a feature of the second pattern of the second test pattern image of the at least one test pattern image produced by the digital projector. In a further variation thereof, the feature of the second pattern may be a line of the second test pattern image produced with the digital projector of the illumination system. In still another variation thereof, an offset of the line from an edge of the image may be adjusted.

In another example thereof, the medical device the controller may be further configured to perform the following step of adjusting at least one characteristic of the observation system based on the determined first movement of the eye. In a variation thereof, the step of adjusting the at least one characteristic of the observation system based on the determined movement of the eye includes adjusting a focal plane of the observation system.

In a further example thereof, the controller may be further configured to perform the following steps determining a second movement of the eye causes at least a portion of the scan definition to be outside of a scan envelope of the examination system with the illumination system and the observation system in the first spatial configuration; and adjusting a position of the illumination system and a position of the observation system of the examination system to a second spatial configuration relative to the eye whereat the portion of the scan definition may be within the scan envelope of the examination system, the second spatial configuration being different than the first spatial configuration. In a variation thereof, the medical device may further comprise a movable base supporting the illumination system and the observation system; and a patient support adapted to support the eye of the patient. In another variation thereof, the controller may be further configured to perform the step of adjusting a position of the movable base relative to the patient support to position the illumination system and the observation system in the second spatial configuration relative to the eye.

In a further exemplary embodiment of the present disclosure, a medical device for determining a separation from an eye. The medical device comprising: an optical system positioned to receive reflected light from the eye, the optical system having an optical axis; an image sensor positioned to detect an image formed by the optical system, the image sensor tilted relative to the optical axis of the optical system such that a first portion of the image sensor on a first side of a plane passing through the optical axis may be closer to a rear face of the optical system than a second portion of the image sensor on a second side of the plane passing through the optical axis, the second side of the plane being opposite the first side of the plane; and a controller configured to receive image data of the detected image from the image sensor and to determine the separation from the eye based on analyzing multiple portions of image data including at least one of a first portion of the image data from the first portion of the image sensor and a second portion of the image data from the second portion of the image sensor.

In an example thereof, the controller may further analyze at least a third portion of the image data, the third portion of image data being closer to the plane than the at least one of the first portion of the image data from the first portion of the image sensor and the second portion of the image data from the second portion of the image sensor.

In another example thereof, the controller may analyze both the first portion of the image data from the first portion of the image sensor and the second portion of the image data from the second portion of the image sensor.

In still another example thereof, the controller may analyze a frequency component of the image data to determine the separation from the eye. In a variation thereof, the frequency component may be a frequency domain component.

In yet another example thereof, the controller may be configured to: (a) convert the image data to a grayscale image data; (b) determine a frequency domain of the grayscale image data; (c) apply a first high pass filter to the frequency domain of the grayscale image to produce a high frequency domain image; (d) apply a second high pass filter to the frequency domain of the grayscale image to produce a mid-to-high frequency domain image; (e) determine a first spatial image of the high frequency domain image; (f) determine a second spatial image of the mid-to-high frequency domain image; (g) for each pixel of the image data determine a ratio of the pixel value in the first spatial image to the pixel value in the second spatial image to produce a focus map; (h) determine a centroid of the focus map; and (i) based on a position of the centroid determine the separation from the eye. In a variation thereof, the separation from the eye may be determined by determining a pixel separation of the centroid from the plane. In a further variation thereof, the pixel separation may be converted to a distance.

In yet another exemplary embodiment of the present disclosure, a medical device for determining a separation from an eye may be provided. The medical device may comprise an optical system positioned to receive reflected light from the eye, the optical system having an optical axis and at least one adjustable lens; an image sensor positioned to detect a plurality of images formed by the optical system, each image of the plurality of images at a different focal length of the optical system; and a controller configured to receive image data of the detected plurality of images from the image sensor and to determine the separation from the eye based on analyzing the plurality of images.

In an example thereof, the controller may analyze a frequency component of the image data of each of the plurality of images to determine the separation from the eye. In a variation thereof, the frequency component may be a frequency domain component.

In another example thereof, the controller may be configured to determine a sharpness characteristic for each image of the plurality of images. In a variation thereof, the controller may determine the sharpness characteristic for each image of the plurality of images by: (a) converting the image data to a grayscale image data; (b) determining a frequency domain of the grayscale image data; (c) applying a first high pass filter to the frequency domain of the grayscale image to produce a high frequency domain image; (d) applying a second high pass filter to the frequency domain of the grayscale image to produce a mid-to-high frequency domain image; (e) determining a first spatial image of the high frequency domain image; (f) determining a second spatial image of the mid-to-high frequency domain image; (g) determine a characteristic based on the first spatial image and the second spatial image; and (h) based on the characteristic based on the first spatial image and the second spatial image determining the separation from the eye. In a variation thereof, the characteristic based on the first spatial image and the second spatial image may be a ratio of a summation of each pixel value in the first spatial image to a summation of each pixel value in the second spatial image. In another variation thereof, a first image of the plurality of images may be selected based on having an extreme value of the characteristic based on the first spatial image and the second spatial image and the separation from the eye may be determined based on a characteristic of the at least one adjustable lens corresponding to the first image.

In yet another example thereof, the controller may determine the sharpness characteristic for each image of the plurality of images by: (a) converting the image data to a grayscale image data; (b) normalize the image data; (c) determining a characteristic of the normalized image data; and (d) based on the characteristic of the normalized image data determining the separation from the eye. In a variation thereof, the step of determining the characteristic of the normalized image data may include the steps of: determining a convolution of the image data with an edge detection kernel; and summing the pixel values resulting from the step of determining the convolution of the image data with the edge detection kernel. In another variation thereof, the step of determining the characteristic of the normalized image data may include the step of determining a variance characteristic of the normalized image data. In a further variation thereof, a first image of the plurality of images may be selected based on having an extreme value of the characteristic of the normalized image data and the separation from the eye may be determined based on a characteristic of the at least one adjustable lens corresponding to the first image. In still another variation thereof, the extreme value of the characteristic of the normalized image data may be a maximum value of the characteristic of the normalized image data relative to a remainder of the plurality of images.

In yet still a further example thereof, the controller may be further configured to determine based on the plurality of images a focal depth range of the medical device. In a variation thereof, the focal depth range may be determined by determining a characteristic of each image of the plurality of images and based on the determined characteristic of each image determining the separation from the eye. In another variation thereof, the controller may be further configured to determine a plurality of locations for illumination of the eye by the medical device, the plurality of locations being within the focal depth range. In a further variation thereof, the controller may be further configured to determine a plurality of locations for imaging of the eye by the medical device, the plurality of locations being within the focal depth range.

In another still example thereof, the at least one adjustable lens of the optical system includes at least one tunable lens.

In yet another still example thereof, the at least one adjustable lens of the optical system includes at least one movable lens.

In a further still example thereof, the medical device may further comprise an illumination system including a digital projector producing at least one test pattern image to illuminate the eye and a first adjustable optical assembly to project the at least one test pattern image towards the eye; and an observation system including the optical system which may be positioned to receive imaging rays produced by reflection of light from the eye of the patient due to the projection of the at least one test pattern image towards the eye by the illumination system and a first camera configured receive the imaging rays from the second adjustable optical assembly, the illumination system and the observation system being positioned in a first spatial configuration relative to the eye. In a variation thereof, the at least one test pattern may include a vertical slit and in a first image of the at least one test pattern image the vertical slit may be in a first transverse position and in a second image of the at least one test pattern image the vertical slit may be in a second transverse position, the second traverse position in second image being spaced apart from the first transverse position in the first image. In another variation thereof, the controller may focus the first adjustable optical assembly of the illumination system at a first focal depth of a plurality of focal depths when the first image of the at least one test pattern image may be produced by the digital projector to focus the vertical slit in the first transverse position on a cornea of the eye at a first location offset from an apex of the cornea of the eye and may focus the first adjustable optical assembly of the illumination system at a second focal depth of the plurality of focal depths when the second image of the at least one test pattern image may be produced by the digital projector to focus the vertical slit in the second transverse position on the cornea at a second location offset from the apex of the cornea, the second location on the cornea being offset from the first location on the cornea. In another variation thereof, the controller may further focus the optical system of the observation system at the first focal depth of the plurality of focal depths when the first image of the at least one test pattern image may be produced by the digital projector to focus the vertical slit in the first transverse position on the cornea at the first location offset from the apex of the cornea and may focus the optical system of the observation system at the second focal depth of the plurality of focal depths when the second image of the at least one image may be produced by the digital projector to focus the vertical slit in the second transverse position on the cornea at the second location offset from the apex of the cornea. In still another variation thereof, the controller may be configured to adjust the first adjustable optical assembly of the illumination system and the optical system of the observation system to a plurality of focal depths relative to an apex of a cornea of the eye while the illumination system and observation system are in the first spatial configuration, wherein a first subset of the plurality of focal depths are part of a first contour and a second subset of the plurality of focal depths are part of a second contour, the second contour being offset relative to the first contour, and wherein the digital projector produces the first test pattern image having the vertical slit in the first transverse position for a first focal depth of the first subset of the plurality of focal depths of the first contour and a first focal depth of the second subset of the plurality of focal depths of the second contour and the digital projector produces the second test pattern image having the vertical slit in the second transverse position for a second focal depth of the first subset of the plurality of focal depths and a second focal depth of second subset of the plurality of focal depths. In still another variation thereof, the first contour may have a first shape and the second contour may have a second shape, the second shape being different than the first shape. In a further variation thereof, the second shape may be flatter than the first shape.

In yet a further example thereof, the medical device may further comprise a movable base. The illumination system may be supported by the movable base and the observation system may be supported by the movable base. The illumination system may be movable relative to the movable base. The illumination system may be rotatable relative to the observation system about a first rotation axis. In a variation thereof, the controller may be further configured to determine a movement of the eye during an examination and to alter a position of the movable base based on the movement of the eye. In another variation thereof, the medical device may further comprise a patient support adapted to position the eye of the patient, wherein the movable base may be moveable relative to the patient support.

In still another exemplary embodiment of the present disclosure, a medical device for imaging at least a portion of an eye of a patient may be provided. The medical device comprising: an illumination system including a digital projector producing at least one test pattern image to illuminate the eye and a first adjustable optical assembly to project the at least one test pattern image towards the eye; an observation system including a second adjustable optical assembly positioned to receive imaging rays produced by reflection of light from the eye of the patient due to the projection of the at least one test pattern image towards the eye by the illumination system and a first camera configured receive the imaging rays from the second adjustable optical assembly; a movable base supporting the illumination system and the observation system; a patient support adapted to position the eye of the patient, wherein the movable base may be moveable relative to the patient support; and a controller operatively coupled to the illumination system, the observation system, and the movable base, the controller configured to adjust the first adjustable optical assembly of the illumination system and the second adjustable optical system of the observation system to a plurality of focal depths relative to the eye with the movable base in a first position relative to the patient support and the controller further configured to adjust the second adjustable optical system of the observation system to a second plurality of focal depths with the movable base in a second position relative to the patient support, the second position being different than the first position, the second position being further from the patient support than the first position, the second plurality of focal depths each providing a respective overall image of the eye to be captured by the first camera.

In an example thereof, the first plurality of focal depths may each provide a respective image of a portion of the eye to be captured by the first camera. In a variation thereof, each of the respective overall images of the eye may include a complete extent of the eye.

In a further exemplary embodiment of the present disclosure, a medical device for imaging at least a portion of an eye of a patient is provided. The medical device comprising an illumination system including a digital projector producing at least one test pattern image to illuminate the eye and a first optical assembly to project a first test pattern image of the at least one test pattern image. The first test pattern image may have an overall width and an overall height. The first test pattern image may include a first region having a first intensity, a second region having a second intensity greater than the first intensity, and a third region having a third intensity greater than the second intensity. The medical device may further comprise an observation system including a second optical assembly positioned to receive imaging rays produced by reflection of light from the eye of the patient due to the projection of the first test pattern image towards the eye by the illumination system and a first camera configured receive the imaging rays from the second optical assembly and a controller operatively coupled to the illumination system and to the observation system. The controller may be configured to adjust a focal depth of the illumination system and a focal depth of the observation system.

In an example thereof, the first intensity is zero. In another example thereof, the first intensity is greater than zero.

In a further example thereof, the second intensity of the second region may be less than 75 percent of the third intensity of the third region.

In still another example thereof, the second intensity of the second region may be less than 50 percent of the third intensity of the third region.

In yet another example thereof, the second intensity of the second region may be less than 30 percent of the third intensity of the third region.

In a further example thereof, the first test pattern image may include a fourth region having a fourth intensity value. The fourth region may be positioned between the second region and the third region. The fourth intensity value may be less than the second intensity value. In a variation thereof, the fourth intensity value may be generally equal to the first intensity value.

In still a further example thereof, the first test pattern image may include a fifth region having a fifth intensity value. The second region may be positioned between the third region and the fifth region. The fifth intensity value may be greater than the second intensity value. In a variation thereof, the fifth intensity value may be generally equal to the third intensity value. In another variation thereof, the first test pattern image may include a sixth region having a sixth intensity value. The sixth region may be positioned between the second region and the fifth region. The sixth intensity value may be less than the second intensity value. In a further variation thereof, the sixth intensity value may be generally equal to the first intensity value.

In yet still a further example thereof, the first test pattern image includes a seventh region having a seventh width. The seventh region may be positioned between the first region and the second region. The seventh region having an intensity profile along the seventh width which transitions from the first intensity value of the first region to the second intensity value of the second region. In a variation thereof, the intensity profile of the seventh region may be a linear profile.

In another example thereof, the first region has a first height, the second region has a second height, and the third region has a third height. The second height of the second region may be generally equal to the overall height of the first test pattern image and the third height of the third region may be generally equal to the overall height of the first test pattern image. In a variation thereof, at least one of (a) the second intensity may be generally constant along the second height of the second region and (b) the third intensity may be generally constant along the third height of the third region. In another variation thereof, at least one of (a) the second intensity may be part of a second intensity profile of the second region that has a first intensity value at a first height position along the second height and a second intensity value at a second height position along the second height spaced apart from the first height position, the second intensity value being less than the first intensity value and (b) the third intensity may be part of a third intensity profile of the third region that has a third intensity value at a third height position along the third height and a fourth intensity value at a fourth height position along the third height spaced apart from the third height position, the fourth intensity value being less than the third intensity value. In a further variation thereof, the controller may be further configured to determine a portion of the first test pattern image corresponding to a first anatomical region of the eye. In yet a further variation thereof, at least one of (a) both the first height position along the second height and the second height position along the second height are positioned within or on a boundary of the portion of the first test pattern image corresponding to the first anatomical region of the eye and (b) both the third height position along the third height and the fourth height position along the third height are positioned within or on a boundary of the portion of the first test pattern image corresponding to the first anatomical region of the eye. In another variation thereof, at least one of (a) the first height position along the second height may be positioned within or on a boundary of the portion of the first test pattern image corresponding to the first anatomical region of the eye and the second height position along the second height may be positioned outside of the portion of the first test pattern image corresponding to the first anatomical region of the eye and (b) the third height position along the third height may be positioned within or on a boundary of the portion of the first test pattern image corresponding to the first anatomical region of the eye and the fourth height may be position outside of the portion of the first test pattern image corresponding to the first anatomical region of the eye. In yet another variation thereof, the portion of the first test pattern image corresponds to a region bounded by the limbus of the eye.

In still a further example thereof, at least the second region of the first test pattern image and the third region of the first test pattern image form a compound slit and the controller may be further configured to determine a portion of the first test pattern image corresponding to a region bounded by the limbus of the eye and to limit a height of the compound slit to the portion of the first test pattern image corresponding to the region bounded by the limbus. In a variation thereof, at least one of the fourth region, the fifth region, the sixth region, and the seventh region are part of the compound slit. In another variation thereof, the compound slit has a generally constant width along a height direction.

In a further still example thereof, at least the second region of the first test pattern image and the third region of the first test pattern image may form a compound slit and the controller may be further configured to determine a portion of the first test pattern image corresponding to a region bounded by the limbus of the eye and to conform the compound slit to generally coincide with a portion of a boundary of the region bounded by the limbus such that the compound slit may be generally arcuate along the height direction.

In another still example thereof, the controller (a) projects the first test pattern image with the illumination system focused at a first focal depth and the observation system focused at a first focal depth, (b) captures at least a first image with the observation system while the first test pattern image may be being projected, (c) projects a second test pattern image different from the first test pattern image with the illumination system focused at a second focal depth and the observation system focused at a second focal depth, and (d) captures at least a second image with the observation system while the second test pattern image may be being projected. In a variation thereof, the second test pattern image includes the third region of the first test pattern image and omits the second region of the first test pattern image. In another variation thereof, the controller combines the at least first image and the at least second image to form a compound image.

In yet a further still example thereof, the controller (a) projects the first test pattern image with the illumination system focused at a first focal depth and the observation system focused at a first focal depth, (b) captures at least a first image with the observation system while the first test pattern image may be being projected, (c) projects a second test pattern image different from the first test pattern image with the illumination system focused at a second focal depth and the observation system focused at a second focal depth, (d) captures at least a second image with the observation system while the second test pattern image may be being projected, (e) projects a third test pattern image different from the first test pattern image with the illumination system focused at a third focal depth and the observation system focused at a third focal depth, and (d) captures at least a third image with the observation system while the second test pattern image may be being projected. In a variation thereof, the third test pattern image may be the same as the second test pattern image. In another variation thereof, the second test pattern image may include the third region of the first test pattern image and may omit the second region of the first test pattern image. In still another variation thereof, the controller may combine the at least first image, the at least second image, and the at least third image to form a compound image. In a further variation thereof, the first focal depth of the illumination system may correspond generally to an anterior surface of a lens of the eye, the second focal depth of the illumination system may correspond generally to a cornea of the eye, and the third focal depth of the illumination system may correspond generally to a posterior surface of the lens of the eye.

In yet another example thereof, the first region has a first height, the second region has a second height, and the third region has a third height. The second height of the second region may be generally equal to the overall height of the first test pattern image and the third height of the third region may be generally equal to the overall height of the first test pattern image.

In still another example thereof, the second region has a second width and the third region has a third width. The second width may be about 0.5 millimeters to about 3.0 millimeters.

In yet a further example thereof, the third region has a third width and a third height, the third width may be about 0.04 millimeters to about 0.8 millimeters.

In still yet another example thereof, the second region has a second width and the third region has a third width. The second width of the second region may be greater than the third width of the third region. In a variation thereof, the second width of the second region may be at least twice the third width of the third region. In another variation thereof, the second width of the second region may be at least thrice the third width of the third region. In a further variation thereof, the second width of the second region may be up to fifty times the third width of the third region.

In yet a further example thereof, the first optical assembly of the illumination system may be a first adjustable optical assembly and the second optical assembly of the observation system may be a second adjustable optical assembly. In a variation thereof, the illumination system and the observation system may be positioned in a first spatial configuration relative to the eye. The controller may be configured to adjust the first adjustable optical assembly of the illumination system and the second adjustable optical system of the observation system to a plurality of focal depths relative to the eye while the illumination system and observation system are in the first spatial configuration.

In still another example thereof, for a first focal depth the controller may be configured to focus the third region of the test pattern image at the first focal depth.

In still another exemplary embodiment thereof, a medical device for imaging at least a portion of an eye of a patient is provided. The medical device comprising an illumination system including a digital projector producing at least one test pattern image to illuminate the eye and a first optical assembly to the at least one test pattern image towards the eye; an observation system including a second optical assembly positioned to receive imaging rays produced by reflection of light from the eye of the patient due to the projection of the first test pattern image towards the eye by the illumination system and a first camera configured receive the imaging rays from the second optical assembly; and a controller operatively coupled to the illumination system and to the observation system. The controller may be configured to receive at least one image of the corneal region of the eye from the observation system. The at least one image of the corneal region of the eye corresponding to the eye may be illuminated with a compound slit produced by the digital projector of the illumination system. The compound slit may have a first region with a first intensity and a second region with a second intensity. The second intensity being less than the first intensity. An area of the second region being greater than an area of the first region.

In an example thereof, the controller may be further configured to receive multiple images of the conjunctiva of the eye from the observation system. Each image may correspond to a respective gaze of the eye. In a variation thereof, the controller may be further configured to combine the multiple images of the conjunctiva of the eye and the at least one image of the corneal region of the eye. In another variation thereof, the multiple images of the conjunctiva of the eye may be combined to form an image of the conjunctiva that surrounds the corneal region. In a further variation thereof, the controller may receive an input to alter at least one of (a) a position of the compound slit, (b) a first characteristic of the compound slit; (c) a position of at least one of the illumination system and the observation system to place the illumination system and the observation system in a second spatial configuration; and (d) a focal depth of at least one of the illumination system and the observation system.

In another example thereof, the first region of the compound slit abuts the second region of the compound slit.

In a further example thereof, the first region of the compound slit may be spaced apart from the second region of the compound slit.

In yet another example thereof, each of the first region of the compound slit and the second region of the compound slit may be generally rectilinear in shape.

In still another example thereof, each of the first region of the compound slit and the second region of the compound slit extend outside of the corneal region of the eye.

In yet still another example thereof, each of the first region of the compound slit and the second region of the compound slit may be confined generally to the corneal region of the eye.

In a further still example thereof, for a first focal depth the controller may be configured to focus the first region of the test pattern image at the first focal depth.

In yet still another exemplary embodiment thereof, a medical device for imaging at least a portion of an eye of a patient is provided. The medical device comprising an illumination system including a digital projector producing at least one test pattern image to illuminate the eye and a first optical assembly to the at least one test pattern image towards the eye; an observation system including a second optical assembly positioned to receive imaging rays produced by reflection of light from the eye of the patient due to the projection of the first test pattern image towards the eye by the illumination system and a first camera configured receive the imaging rays from the second optical assembly; and a controller operatively coupled to the illumination system and to the observation system. The controller may be configured to receive a first image of the corneal region of the eye corresponding to the eye being illuminated with a first portion of a compound slit produced by the digital projector of the illumination system, receive a second image of the corneal region of the eye corresponding to the eye being illuminated with a second portion of a compound slit produced by the digital projector of the illumination system, and combine the first image and the second image to produce an image of the eye illuminated by both the first portion of the compound slit and the second portion of the compound slit.

In an example thereof, the first portion of the compound slit corresponds to a first region with a first intensity and the second portion of the compound slit corresponds to a second region with a second intensity. The second intensity may be less than the first intensity. An area of the second region being greater than an area of the first region. In a variation thereof, the first region of the compound slit abuts the second region of the compound slit. In another variation thereof, the first region of the compound slit may be spaced apart from the second region of the compound slit.

In another example thereof, each of the first region of the compound slit and the second region of the compound slit may be generally rectilinear in shape.

In a further example thereof, each of the first region of the compound slit and the second region of the compound slit may extend outside of the corneal region of the eye.

In still a further example thereof, each of the first region of the compound slit and the second region of the compound slit may be confined generally to the corneal region of the eye.

In another example thereof, the medical device may further comprise a secondary illumination system including an illumination source and optics to project an output of the illumination source towards the eye. In a variation thereof, the illumination source of the secondary illumination system may be one of a laser diode, a fiber coupled diode laser, and a light emitting diode. In another variation thereof, the illumination source of the secondary illumination system may emit a white illumination, a red illumination, or a near infrared illumination. In still another variation thereof, a first optical axis directed at the eye of the illumination system and a second optical axis directed at the eye of the secondary illumination system may fixed relative to each other. In yet still another variation thereof, the illumination system and the secondary illumination system may rotate together relative to the eye. In a first rotational position of illumination system and the secondary illumination system the illumination system may be aligned with the eye. In a second rotational position of the illumination system and the secondary illumination system the secondary illumination system may be aligned with eye. In a further variation thereof, the first optical axis directed at the eye of the illumination system and the second optical axis directed at the eye of the secondary illumination system may be angled relative to each other. Optionally, the first optical axis directed at the eye of the illumination system may be angled at about 10 degrees to the second optical axis directed at the eye of the secondary illumination system. In a further still variation thereof, in the second rotational position of the illumination system and the secondary illumination system the illumination system may be outside of a field of view of the first camera and the optics of the secondary illumination system may be within the field of view of the first camera.

In a further still exemplary embodiment of the present disclosure, a medical device for imaging at least a portion of an eye of a patient is provided. The medical device may comprise an illumination system including a digital projector producing at least one test pattern image to illuminate the eye and a first adjustable optical assembly to project the at least one test pattern image towards the eye and a secondary illumination system including an illumination source and optics to project an output of the illumination source towards the eye. The medical device may further comprise an observation system including a second adjustable optical assembly positioned to receive imaging rays produced by reflection of light from the eye of the patient due to the projection of the at least one test pattern image towards the eye by the illumination system when the illumination system, the secondary illumination system, and the observation system are positioned in a first spatial configuration relative to the eye and to receive imaging rays produced by reflection of light from the eye of the patient due to the projection of the output of the illumination source of the secondary illumination system when the illumination system, the secondary illumination system, and the observation system are positioned in a second spatial configuration relative to the eye. The observation system may further include a first camera configured to receive the imaging rays from the second adjustable optical assembly. The medical device may further comprise a controller operatively coupled to the illumination system, the secondary illumination system, and to the observation system. The controller may be configured to adjust the first adjustable optical assembly of the illumination system and the second adjustable optical system of the observation system to a plurality of focal depths relative to the eye while the illumination system, the secondary illumination system, and observation system are in the first spatial configuration.

In an example thereof, the illumination source of the secondary illumination system may be one of a laser diode, a fiber coupled diode laser, and a light emitting diode.

In another example thereof, the illumination source of the secondary illumination system may emit a white illumination, a red illumination, or a near infrared illumination.

In yet another example thereof, a first optical axis directed at the eye of the illumination system and a second optical axis directed at the eye of the secondary illumination system may be fixed relative to each other. In a variation thereof, the illumination system and the secondary illumination system may rotate together relative to the eye. In a first rotational position of illumination system and the secondary illumination system the illumination system may be aligned with the eye. In a second rotational position of the illumination system and the secondary illumination system the secondary illumination system may be aligned with eye. In a further variation thereof, the first optical axis directed at the eye of the illumination system and the second optical axis directed at the eye of the secondary illumination system may be angled relative to each other. Optionally the first optical axis directed at the eye of the illumination system may be angled at about 10 degrees to the second optical axis directed at the eye of the secondary illumination system. In still another variation thereof, in the second rotational position of the illumination system and the secondary illumination system the illumination system may be outside of a field of view of the first camera and the optics of the secondary illumination system may be within the field of view of the first camera.

In another still exemplary embodiment of the present disclosure, a medical device for imaging at least a portion of an eye of a patient is provided. The medical device may comprise a first illumination system including a first illumination source and a second illumination system including a second illumination source. The first illumination system may produce a first output to illuminate the eye. The first illumination system may include a first optical assembly to project the first output towards the eye. The second illumination system may produce a second output to illuminate the eye. The medical device may further comprise an observation system including a second optical assembly positioned to receive imaging rays produced by reflection of light from the eye of the patient due to the first output of the first illumination system when the first illumination system, the second illumination system, and the observation system are positioned in a first spatial configuration relative to the eye and to receive imaging rays produced by reflection of light from the eye of the patient due to the second output of the second illumination system when the first illumination system, the second illumination system, and the observation system are positioned in a second spatial configuration relative to the eye. The second spatial configuration may be different than the first spatial configuration. Both the first illumination system and the second illumination system may be rotatable relative to the observation system. The observation system may further include a first camera configured to receive the imaging rays from the second adjustable optical assembly. The medical device may further comprise a controller operatively coupled to the first illumination system, the second illumination system, and to the observation system. The controller may be configured to selectively illuminate the eye with the first illumination system in the first spatial configuration and to illuminate the eye with the second illumination system in the second spatial configuration.

In an example thereof, a first optical axis directed at the eye of the first illumination system and a second optical axis directed at the eye of the second illumination system may be fixed relative to each other. In a variation thereof, the first optical axis directed at the eye of the first illumination system and the second optical axis directed at the eye of the second illumination system may be angled relative to each other. Optionally the first optical axis directed at the eye of the first illumination system may be angled at about 10 degrees to the second optical axis directed at the eye of the second illumination system. In a further variation thereof, the first illumination system and the second illumination system may rotate together relative to the eye. In still another variation thereof, in the second spatial configuration the first illumination system may be outside of a field of view of the first camera of the observation system and a portion of the second illumination system may be within the field of view of the first camera. In yet still another variation thereof, the first illumination system may include a first adjustable optical assembly and the observation system may include a second adjustable optical assembly. The controller may be configured to adjust the first adjustable optical assembly of the first illumination system and the second adjustable optical system of the observation system to a plurality of focal depths relative to the eye while the first illumination system, the second illumination system, and observation system are in the first spatial configuration. In still another variation thereof, the second illumination source of the second illumination system may be one of a laser diode, a fiber coupled diode laser, and a light emitting diode. In yet still another example thereof, the second illumination source of the second illumination system may emit a white illumination, a red illumination, or a near infrared illumination.

In still yet another exemplary embodiment of the present disclosure, a medical device for imaging at least a portion of an eye of a patient is provided. The medical device may comprise an observation system including a first camera and an illumination system including a first illumination source. The observation system may image the eye along a first axis. The first axis may intersect the eye at a first location. The first illumination system may produce a first output to illuminate the eye and a first optical assembly to project the first output towards the eye. The first output may be projected to intersect the eye at a second location spaced apart from the first location. A portion of the first illumination system may be within a field of view of the first camera of the observation system.

In an example thereof, the portion may be a first portion of the first optical assembly of the illumination system. In a variation thereof, the first optical assembly may expand an output of the first illumination source and may focus the expanded output at the second location. In another variation thereof, the first location may correspond to an apex of a cornea of the eye and the second location may correspond to a portion of the cornea of the eye offset from the first axis. In a further variation thereof, the first output may form an illuminated area on a retina of the eye, the illuminated area may include the first axis. In still another variation thereof, the second location may be offset from the first location by about 1 millimeter. In yet another variation thereof, the first output may be angled relative to the first axis. In still a further variation thereof, the first output may be angled relative to the first axis by about 1 to about 3 degrees. In still yet a further variation thereof, the first illumination source may be one of a laser diode, a fiber coupled diode laser, and a light emitting diode. In another still variation thereof, the first illumination source may emit a white illumination, a red illumination, or a near infrared illumination.

In another still exemplary embodiment of the present disclosure, a medical device for imaging at least a portion of an eye of a patient is provided. The medical device may comprise an observation system including a first camera and an illumination system may include a first illumination source. The observation system may image the eye along a first axis. The first axis may intersect the eye at a first location corresponding to an apex of a cornea of the eye. The first illumination system may produce a first output to illuminate the eye and a first optical assembly to project the first output towards the eye. The first output may project to intersect the eye at a second location spaced apart from the first location. The first output may be focused at the second location to form an illuminated area on a retina of the eye. The illuminated area may include the first axis.

In an example thereof, the second location is offset from the first location by about 1 millimeter. In a variation thereof, the first output may be angled relative to the first axis. In another variation thereof, the first output may be angled relative to the first axis by about 1 to about 3 degrees. In a further variation thereof, the first illumination source may be one of a laser diode, a fiber coupled diode laser, and a light emitting diode. In another still variation thereof, the first illumination source may emit a white illumination, a red illumination, or a near infrared illumination.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive. Other aspects and optional and/or preferred embodiments will become apparent from the following description provided below with reference to the accompanying drawings.

### BRIEF DESCRIPTION OF THE DRAWINGS

The above-mentioned and other features and advantages of this disclosure, and the manner of attaining them, will become more apparent and will be better understood by reference to the following description of exemplary embodiments taken in conjunction with the accompanying drawings, wherein:

FIG. 1 illustrates a schematic view showing the basic components of the human eye;

FIG. 1A illustrates an enlarged view of a portion of FIG. 1;

FIG. 2 illustrates an exemplary examination system of the present disclosure with an optical axis of an illumination system being angled relative to an optical axis of an observation system;

FIG. 3 illustrates the examination system of FIG. 2 with the optical axis of the illumination system being generally aligned with the optical axis of the observation system;

FIG. 4 illustrates a side view of an exemplary embodiment of the examination system of FIG. 2;

FIG. 5 illustrates a top view of the examination system of FIG. 4;

FIG. 6 illustrates an exemplary illumination system of FIG. 2;

FIG. 7 illustrates an exemplary observation system of FIG. 2 including a first camera and a second camera;

FIG. 7A illustrates another exemplary observation system of FIG. 2 including a first camera, a second camera, and a third camera;

FIG. 8 illustrates a representation of the imaging axes of the first camera and the second camera of FIG. 7;

FIG. 9A illustrates an exemplary image for projection by a projector of an embodiment of the examination system of FIG. 6;

FIG. 9B illustrates the image of FIG. 9A projected onto an eye under examination;

FIG. 10 illustrates an exemplary method or processing sequence of the present disclosure;

FIG. 11A illustrates an image of the first camera of FIG. 7 of the image of FIG. 9A projected onto the eye under examination as shown FIG. 9B;

FIG. 11B illustrates an image of the second camera of FIG. 7 of the image of FIG. 9A projected onto the eye under examination as shown FIG. 9B;

FIG. 12 illustrates a first portion of the image of FIG. 11A and a second portion of the image of FIG. 11B when the optical axis of the first camera of FIG. 7 and the second camera of FIG. 7 intersect in front of a surface of a cornea of the eye under examination;

FIG. 13 illustrates the first portion of the image of FIG. 11A and the second portion of the image of FIG. 11B when the optical axis of the first camera of FIG. 7 and the second camera of FIG. 7 intersect behind the surface of the cornea of the eye under examination;

FIG. 14 illustrates the first portion of the image of FIG. 11A and the second portion of the image of FIG. 11B when the optical axis of the first camera of FIG. 7 and the second camera of FIG. 7 intersect at the surface of the cornea of the eye under examination;

FIG. 15 illustrates an exemplary processing sequence of the present disclosure;

FIG. 16A illustrates an exemplary image for projection by a projector of an embodiment of the examination system of FIG. 6 of a first position of a single vertical slit;

FIG. 16B illustrates the image of FIG. 16A projected onto an eye under examination and imaged by a camera of the FIG. 7;

FIG. 17A illustrates an exemplary image for projection by a projector of an embodiment of the examination system of FIG. 6 of a second position of a single vertical slit;

FIG. 17B illustrates the image of FIG. 17A projected onto an eye under examination and imaged by a camera of the FIG. 7;

FIG. 18A illustrates an exemplary image for projection by a projector of an embodiment of the examination system of FIG. 6 of a third position of a single vertical slit;

FIG. 18B illustrates the image of FIG. 18A projected onto an eye under examination and imaged by a camera of the FIG. 7;

FIG. 19A illustrates an exemplary image for projection by a projector of an embodiment of the examination system of FIG. 6 of a first position of a double vertical slit;

FIG. 19B illustrates the image of FIG. 19A projected onto an eye under examination and imaged by a camera of the FIG. 7;

FIG. 20A illustrates an exemplary image for projection by a projector of an embodiment of the examination system of FIG. 6 of a first position of a single vertical slit and additional indicia;

FIG. 20B illustrates the image of FIG. 20A projected onto an eye under examination and imaged by a camera of the FIG. 7;

FIGS. 21 and 22 illustrate the focus depths of the illumination system and observation systems for various positions of a vertical slit.

FIG. 22A illustrate focal depths for multiple exemplary contours;

FIGS. 23 and 24 illustrate multiple exemplary contours for an eye examination;

FIG. 25 illustrates multiple exemplary test locations for a plurality of contours for an eye exam;

FIG. 26, illustrates multiple locations of a vertical slit to be projected by an exemplary illumination system during an eye examination;

FIG. 27 illustrates relative positioning of a plurality of test locations on a contour;

FIG. 27A illustrates relative positioning of a plurality of test locations on a contour wherein the contour has moved due to an eye movement;

FIG. 28 illustrates a ray equation corresponding to a projection direction of a vertical slit of an exemplary illumination system and an eye scan contour;

FIG. 29 illustrates an exemplary curvature value for a plurality of contours;

FIG. 30 illustrates a numerical solution to determining a transverse coordinate corresponding to an intersection point of the ray equation and eye scan contour of FIG. 28;

FIGS. 31 and 32 illustrate an exemplary limbus detection method;

FIGS. 33A-B illustrate an exemplary eye movement detection method;

FIG. 34 is a front perspective view of an exemplary examination system;

FIG. 35 is a side view of the exemplary examination system of FIG. 34;

FIG. 36 is a rear perspective view of the exemplary examination system of FIG. 34;

FIG. 37 is a top view of the exemplary examination system of FIG. 34 with the illumination system and observation system at different angular locations;

FIG. 38 is a second top view of the exemplary examination system of FIG. 34 with the illumination system and observation system at the same angular location;

FIG. 39 is a third top view of the exemplary examination system of FIG. 34 with the illumination system and observation system at different angular locations;

FIG. 40 is a cross-sectional view of the exemplary examination system of FIG. 34 along lines 40-40 in FIG. 37;

FIG. 41 is a detail view of a portion of the exemplary examination system of FIG. 34 and the eye of a patient;

FIG. 41A is a representative view of a portion of the exemplary examination system of FIG. 34 with the illumination system axis tilted relative to an observation axis;

FIG. 41B is a representative view of a portion of the exemplary examination system of FIG. 34 with the illumination system having a first illumination source for examining the eye and a second illumination system for retroillumination imaging;

FIG. 41C is a representative view of the second illumination system for retroillumination imaging;

FIG. 41D is an exemplary image of the eye illuminated by the second illumination system for retroillumination imaging;

FIG. 42 is a perspective view of the exemplary examination system of FIG. 34 illustrating a mounting system of the illumination system.

FIG. 43 is an exemplary processing sequence of the present disclosure for performing a scan;

FIG. 44 is a representative view of a scan definition and a scan envelope of an examination system with the examination system aligned relative to an eye of a patient;

FIG. 45 is a representative view of a scan definition and a scan envelope of an examination system with the examination system misaligned relative to an eye of a patient but with all of the scan locations within the scan envelope;

FIG. 46 is a representative view of a scan definition and a scan envelope of an examination system with the examination system misaligned relative to an eye of a patient with only a portion of the scan locations within the scan envelope;

FIG. 47 is another exemplary processing sequence of the present disclosure for performing a scan;

FIGS. 48A and 48B are a further exemplary processing sequence of the present disclosure for performing a scan;

FIGS. 49A and 49B are a yet further exemplary processing sequence of the present disclosure for performing a scan;

FIG. 50 is a representative view of a system for detecting a separation between an object and a medical device, such as the disclosed examination systems;

FIG. 51 is another still processing sequence of the present disclosure;

FIG. 52 is a first image of an eye captured by an image sensor of the system in FIG. 50;

FIG. 53 is a second image of an eye captured by an image sensor of the system in FIG. 50;

FIG. 54 is a third image of an eye captured by an image sensor of the system in FIG. 50;

FIG. 55 is still another processing sequence of the present disclosure;

FIG. 56 is yet still another processing sequence of the present disclosure;

FIG. 57 is a representative view of a portion of an examination system showing a plurality of focal planes at various focal depths;

FIGS. 58A-D are representative images captured by the portion of the examination system at the plurality of focal planes in FIG. 57;

FIG. 59 is an exemplary processing sequence to determine a separation between an eye and a medical device;

FIG. 60 is another exemplary processing sequence to determine a separation between an eye and a medical device;

FIG. 61 is a representative view of a portion of an examination system showing a plurality of focal planes at various focal depths;

FIG. 62 are representative images captured by the portion of the examination system at the plurality of focal planes in FIG. 61;

FIG. 63 is a representative view of a methodology to determine a separation between an eye and a medical device and a focal depth range of the medical device;

FIG. 64 is an exemplary processing sequence to determine a separation between an eye and a medical device;

FIG. 64A is another exemplary processing sequence to determine a separation between an eye and a medical device and a focal depth range of the medical device;

FIG. 65 is a representative view of a portion of an examination system at a first separation relative to the eye;

FIG. 66 is a representative view of a portion of an examination system at a second separation relative to the eye;

FIG. 67 is an exemplary processing sequence for switching between the arrangement of FIG. 65 and the arrangement of FIG. 66;

FIG. 68A illustrates an exemplary test pattern image to be projected by the display area of the projector having a first slit positioned in a first transverse position and having a height limited based on a determined location of an anatomical feature of the eye;

FIG. 68B illustrates the test pattern image of FIG. 68A projected onto the eye;

FIG. 68C illustrates a second exemplary test pattern image to be projected by the display area of the projector having the first slit of FIG. 68A positioned in a second transverse position and having a revised height limit based on the determined location of the anatomical feature of the eye;

FIG. 68D illustrates the test pattern image of FIG. 68C projected onto the eye;

FIG. 69A illustrates an exemplary test pattern image to be projected by the display area of the projector having a first slit positioned in a first transverse position;

FIG. 69B illustrates the test pattern image of FIG. 69A projected onto the eye;

FIG. 69C illustrates an intensity profile of the first slit of FIG. 69A along lines 69C-69C in FIG. 69A;

FIG. 70A illustrates an exemplary test pattern image to be projected by the display area of the projector having a first ring;

FIG. 70B illustrates the test pattern image of FIG. 70A projected onto the eye;

FIG. 71A illustrates an exemplary test pattern image to be projected by the display area of the projector having a first partial ring;

FIG. 71B illustrates the test pattern image of FIG. 71A projected onto the eye;

FIG. 71C illustrates a test pattern image with a partial ring projected onto the eye;

FIG. 72A illustrates an exemplary test pattern image to be projected by the display area of the projector having a first exemplary compound slit in a first transverse position;

FIG. 72B illustrates an intensity profile of the first exemplary compound slit of FIG. 72A along lines 72B-72B in FIG. 72A;

FIG. 72C illustrates a test pattern image with having a first exemplary compound slit projected onto the eye;

FIG. 72D illustrates an exemplary test pattern image to be projected by the display area of the projector having the first exemplary compound slit of FIG. 72A positioned in a first transverse position and having a height limited based on a determined location of an anatomical feature of the eye;

FIG. 73A illustrates an exemplary test pattern image to be projected by the display area of the projector having a second exemplary compound slit in a first transverse position;

FIG. 73B illustrates an intensity profile of the second exemplary compound slit of FIG. 73A along lines 73B-73B in FIG. 73A;

FIG. 74A illustrates an exemplary test pattern image to be projected by the display area of the projector having a third exemplary compound slit in a first transverse position;

FIG. 74B illustrates an intensity profile of the third exemplary compound slit of FIG. 74A along lines 74B-74B in FIG. 74A;

FIG. 75 illustrates an intensity profile of a fourth exemplary compound slit;

FIG. 76 illustrates an intensity profile of a fifth exemplary compound slit;

FIG. 77 illustrates an intensity profile of a sixth exemplary compound slit;

FIG. 78 illustrates an intensity profile of a seventh exemplary compound slit;

FIG. 79 illustrates an exemplary processing sequence to emulate a compound slit;

FIG. 80 illustrates an exemplary processing sequence of the present disclosure;

FIG. 81 illustrates another exemplary processing sequence of the present disclosure;

FIG. 82 illustrates a further exemplary processing sequence of the present disclosure; and

FIG. 83 illustrates yet another exemplary processing sequence of the present disclosure.

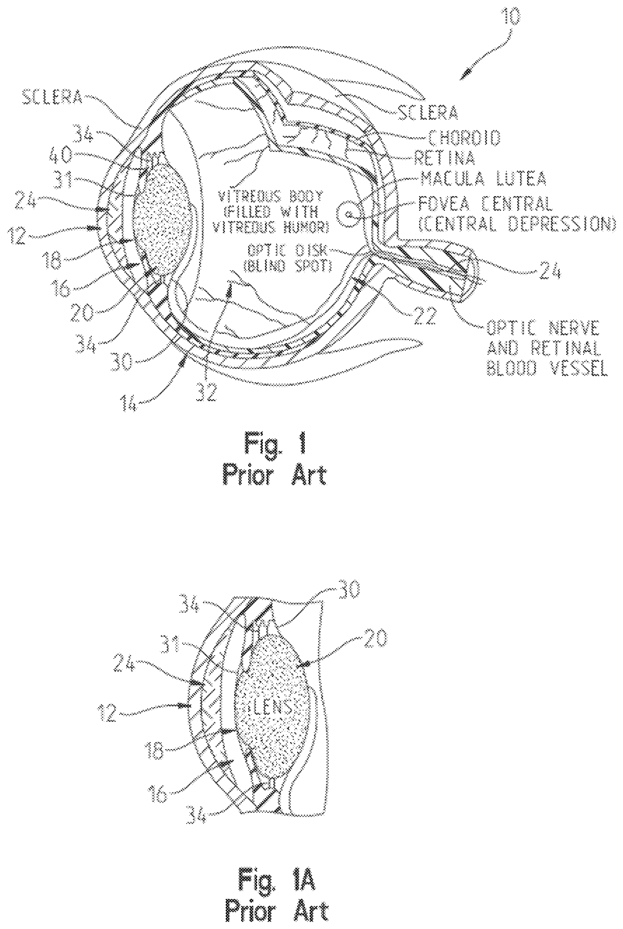

Corresponding reference characters indicate corresponding parts throughout the several views.

While the present disclosure is amenable to various modifications and alternative forms, specific embodiments have been shown by way of example in the drawings and are described in detail below. The intention, however, is not to limit the disclosure to the particular embodiments described.

### DETAILED DESCRIPTION OF THE DRAWINGS

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The terms “about”, “substantially”, “approximately”, “generally” and other terms of approximation include the stated value or condition plus/minus ten percent of the stated value or condition. For example, “about 10 centimeters” should be interpreted to be 9-11 centimeters. As another example, confined generally to a region should be interpreted to mean that region and up to 10% outside of that region.

The term “logic” or “control logic” as used herein may include software and/or firmware executing on one or more programmable processors, application-specific integrated circuits (ASICs), field-programmable gate arrays (FPGAs), digital signal processors (DSPs), hardwired logic, or combinations thereof. Therefore, in accordance with the embodiments, various logic may be implemented in any appropriate fashion and would remain in accordance with the embodiments herein disclosed.

The terms “couples”, “coupled”, “coupler” and variations thereof are used to include both arrangements wherein the two or more components are in direct physical contact and arrangements wherein the two or more components are not in direct contact with each other (e.g., the components are “coupled” via at least a third component), but yet still cooperate or interact with each other.

In some instances throughout this disclosure and in the claims, numeric terminology, such as first, second, third, and fourth, is used in reference to various components or features. Such use is not intended to denote an ordering of the components or features. Rather, numeric terminology is used to assist the reader in identifying the component or features being referenced and should not be narrowly interpreted as providing a specific order of components or features.

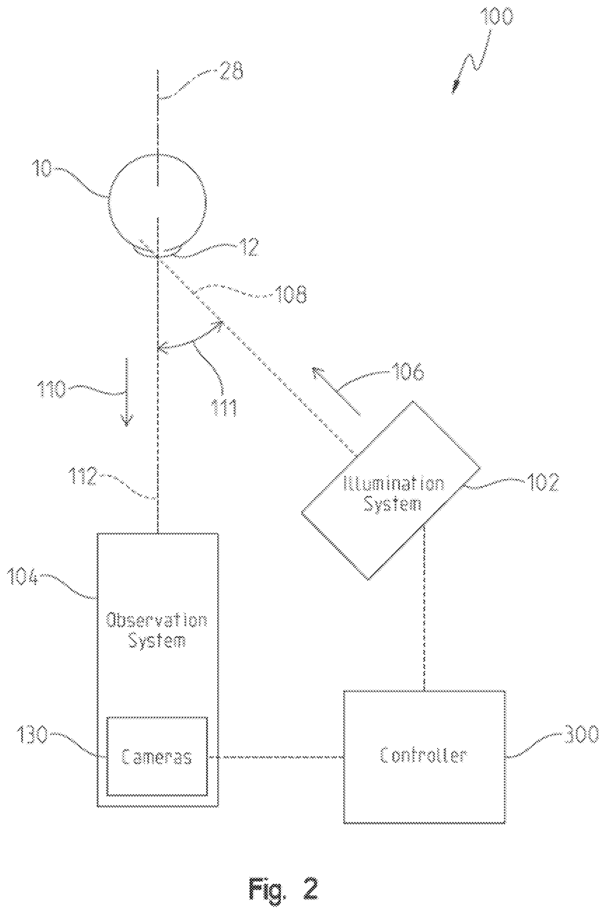

Referring to FIG. 2, an examination system 100 is shown. Examination system 100 includes an illumination system 102 and an observation system 104. Illumination system 102 illuminates eye 10 with light generally in direction 106 along an optical axis 108. Observation system 104 receives reflected light from eye 10 generally in direction 110 along an optical axis 112. The reflected light includes diffuse reflections and may include specular reflections. Unless stated otherwise the terms “reflected”, reflection“, reflects”, and related terms are defined as diffuse reflection or backscattering. As shown in FIG. 2, optical axis 112 is generally aligned with an optical axis 28 of eye 10 and optical axis 108 is angled relative to optical axis 112 by an angle 111. In embodiments, optical axis 108 and optical axis 112 are generally coplanar. Although illumination system 102 and observation system 104 are shown with observation system 104 being positioned directly in front of eye 10 and aligned with axis 28 of the eye 10 and illumination system 102 being angled relative to eye 10, illumination system 102 and observation system 104 may be positioned in any relationship to eye 10. In embodiments, illumination system 102 and observation system 104 are both angled relative to the optical axis 28 of the eye 10. In embodiments, illumination system 102 is positioned directly in front of eye 10 with optical axis 108 aligned with optical axis 28 of eye 10 and observation system 104 is positioned with optical axis 112 angled relative to optical axis 28 of the eye 10.

In embodiments, examination system 100 includes a secondary illumination source (not shown) which illuminates at least portions of the eye. The secondary illumination source may be any light source which provides a generally constant light intensity across a large portion of the eye 10 or bright region at a portion of the eye 10. In embodiments, secondary illumination source may be at a different wavelength than the primary light source of illumination system 102. In one example, the secondary illumination source may be supported by illumination system 102. In one example, the secondary illumination source 114 is separate from illumination system 102.

Observation system 104 includes a plurality of cameras 130. The plurality of cameras 130 each record images of the eye 10 under examination. At least one of the plurality of cameras may record light field data associated with the light reflected from eye 10. The light field data permits refocusing of an image recorded by the at least one camera.

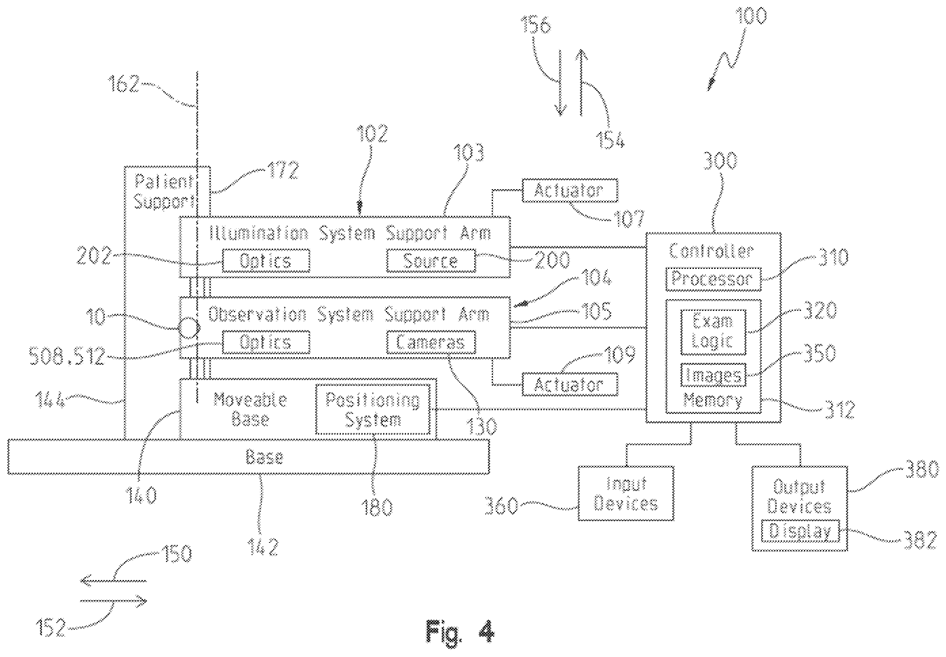

Each of illumination system 102 and observation system 104 may be coupled to a controller 300. In embodiments, controller 300 includes at least one processor 310 and associated memory 312 (see FIG. 4). Memory 312 includes exam logic 320, which monitors the output of cameras 130 and other inputs and sensors disclosed herein to perform one or more operations with examination system 100. The term “logic” as used herein includes software and/or firmware executing on one or more programmable processors, application-specific integrated circuits, field-programmable gate arrays, digital signal processors, hardwired logic, or combinations thereof. Therefore, in accordance with the embodiments, various logic may be implemented in any appropriate fashion and would remain in accordance with the embodiments herein disclosed. A non-transitory machine-readable medium comprising logic can additionally be considered to be embodied within any tangible form of a computer-readable carrier, such as solid-state memory, magnetic disk, and optical disk containing an appropriate set of computer instructions and data structures that would cause a processor to carry out the techniques described herein. This disclosure contemplates other embodiments in which controller 300 is not microprocessor-based, but rather is configured to control operation of examination system 100 based on one or more sets of hardwired instructions. Further, controller 300 may be contained within a single device or be a plurality of devices networked together, wired or wirelessly, or otherwise electrically connected to provide the functionality described herein.

Electronic controller 300 may further receive input through one or more input devices 360 (see FIG. 4). Exemplary input devices include operator actuatable inputs, such as buttons, switches, levers, joysticks, dials, touch displays, and soft keys; sensors; a communication module for connection to a wired or wireless network; and other suitable input devices. Electronic controller 300 may further provide output through one or more output devices 380. Exemplary output devices include visual indicators, audio indicators, a communication module for connection to a wired or wireless network, and other suitable output devices. Exemplary visual indicators include displays, lights, and other visual systems. Exemplary audio indicators include speakers and other suitable audio systems.

Returning to FIG. 4, controller 300 controls one or more settings of illumination system 102 and/or observation system 104 and stores one or more images 350 captured by the plurality of cameras 130. Further, controller 300, in embodiments, controls an operation of one or more of illumination system 102 and observation system 104 with exam logic 320. In embodiments, the positioning of illumination system 102 and observation system 104 relative to the eye 10 is a manual operation. In embodiments, the positioning of illumination system 102 and observation system 104 relative to the eye 10 is controlled by controller 300 either through manual input, such as with an input device 360 which causes controller 300 to reposition actuators to maneuver one or both of illumination system 102 and observation system 104 or through an automated operation of exam logic 320 based on images captured by cameras 130, or a combination of both a manual operation and an automated operation.

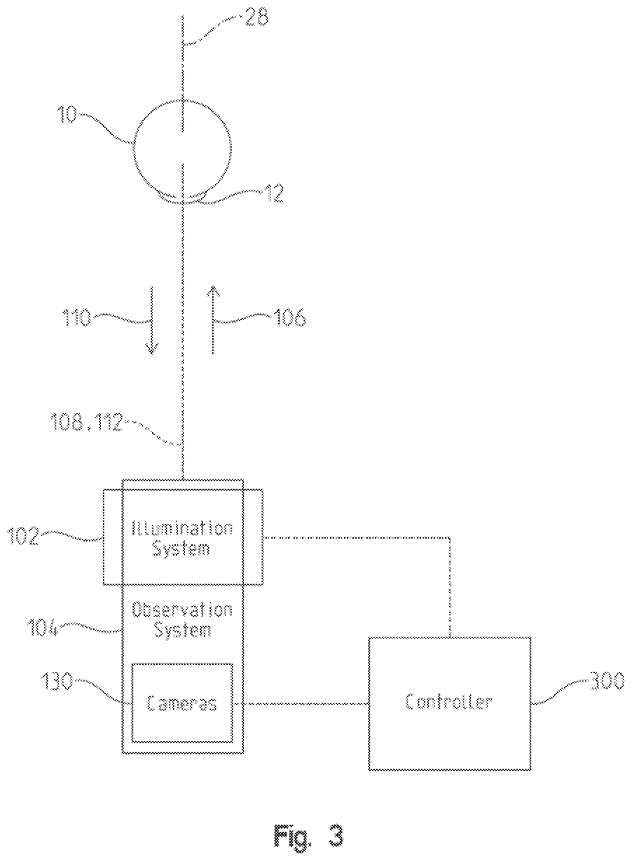

Referring to FIG. 3, examination system 100 is shown wherein optical axis 108 is generally coaxial with optical axis 112 and with optical axis 28 of the eye 10. Thus, observation system 104 is generally in line with illumination system 102. Although illumination system 102 and observation system 104 are shown being positioned directly in front of eye 10 and aligned with optical axis 28, illumination system 102 and observation system 104 may be angled relative to optical axis 28 of the eye 10, such as the position of illumination system 102 in FIG. 2. Further, the optical axis 108 of illumination system 102 and the optical axis 112 of observation system 104 may be parallel with optical axis 28 of the eye, but offset from the optical axis 28 of the eye 10.

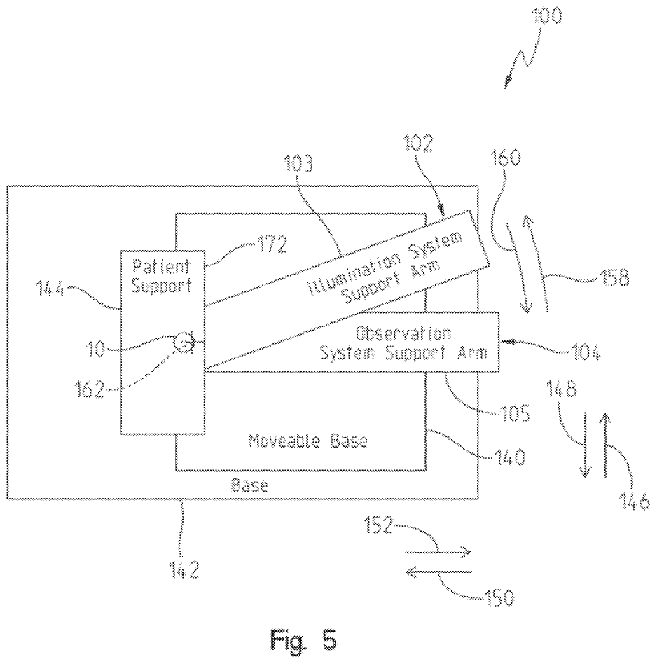

Referring to FIGS. 4 and 5, an exemplary embodiment of examination system 100 is shown. Illumination system 102 and observation system 104 are shown supported by a moveable base 140 which is supported on top of a base 142. In embodiments, base 142 is the floor, a table-top, or an intermediate base which is supported by the floor or tabletop or other structure in an examination room. A patient support 144 is also supported by base 142. Patient support 144 positions an eye 10 of a patient relative to illumination system 102 and observation system 104.

Referring to FIG. 5, moveable base 140 is generally moveable in an x-axis in direction 146 and direction 148 relative to base 142 and in a z-axis in direction 150 and direction 152 relative to base 142. The movement of moveable base 140 relative to base 142 results in the movement of both illumination system 102 and observation system 104. In embodiments, one of illumination system 102 and observation system 104 is not supported by moveable base 140 and thus does not move in concert with moveable base 140 when moveable base 140 is moved relative to base 142.

In embodiments, examination system 100 does not include a base 142 and patient support 144 is a head worn apparatus, similar to goggles. A movable base 140 may be movable mounted to patient support 144 to move illumination system 102 and/or observation system 104 relative to patient support 144. In embodiments, a patient is not supported by patient support 144 and illumination system 102 and observation system 104 are positionable relative to eye 10, such as by an articulating arm supporting both illumination system 102 and observation system 104 or a pair of articulating arms, one supporting illumination system 102 and one supporting observation system 104. The articulating arms may be robotic arms which are controlled by controller 300 to position illumination system 102 and observation system 104 relative to eye 10 of the patient for examination. An advantage, among others, of this arrangement is for patients that are bedridden. In examples, the illumination system 102 and observation system 104 are aligned to the apex of the cornea 12 of the eye 10 of the patient.

Returning to FIG. 4, in embodiments, illumination system 102 and observation system 104 are both moveable relative to moveable base 140 in a y-axis in direction 154 and direction 156 as illustrated in FIG. 4. In embodiments, illumination system 102 and observation system 104 are fixed relative to moveable base 140 in the y-axis and patient support 144 is movable relative to illumination system 102, observation system 104, and moveable base 140 in the y-axis. In embodiments, patient support 144 is supported on base 142 independent of moveable base 140 and may include a head support that is moveable in the y-axis in directions 154, 156.

Further, each of illumination system 102 and observation system 104 are rotatable relative to moveable base 140 in direction 158 and direction 160 as illustrated in FIG. 5. In the illustrated embodiment, each of illumination system 102 and observation system 104 are rotatable about an axis 162. As shown in FIG. 4, axis 162 may be positioned within an envelope 170 of patient support 144 and, in particular, rearward of a leading edge 172 of patient support 144. Illumination system 102 and observation system 104 are individually rotatable relative to moveable base 140. As such, illumination system 102 may be rotated relative to moveable base 140 without a corresponding rotation of observation system 104 relative to moveable base 140 or vice versa.

Although illumination system 102 and observation system 104 are shown being rotatable about a vertical axis, axis 162, one or both of illumination system 102 and observation system 104 may be rotatable about a horizontal axis parallel to the x-axis or another axis in a plane defined by the x-axis and the y-axis. In embodiments, each of illumination system 102 and observation system 104 is rotatable about a separate axis relative to moveable base 140.

Returning to FIG. 4, illumination system 102 is illustrated as including a source 200 and an optics assembly 202 supported by illumination system support arm 103. Similarly, the plurality of cameras 130 and associated optics, such as optics 508, 512, of observation system 104 are supported by an observation system support arm 105. Returning to illumination system 102, in embodiments, source 200 is a mechanical slit forming device having an illumination source and an adjustable width aperture which provides a generally rectilinear slit of light from the illumination source to optical assembly 202. In embodiments, source 200 is a projector. An exemplary projector is a digital light projector (DLP). A digital light projector electronically generates an image that is projected from a plurality of light sources, such as the pixels of a display screen. As described herein, the digital light projector may be used to simulate a slit for a slit lamp examination of eye 10 and the slit may be moved across the eye 10 by altering the image that is projected. Further, portions of an image projected by the digital light projector may be used to track eye movement. Additionally, portions of an image projected by the digital light projector may be used to align the eye 10 to observation system 104.

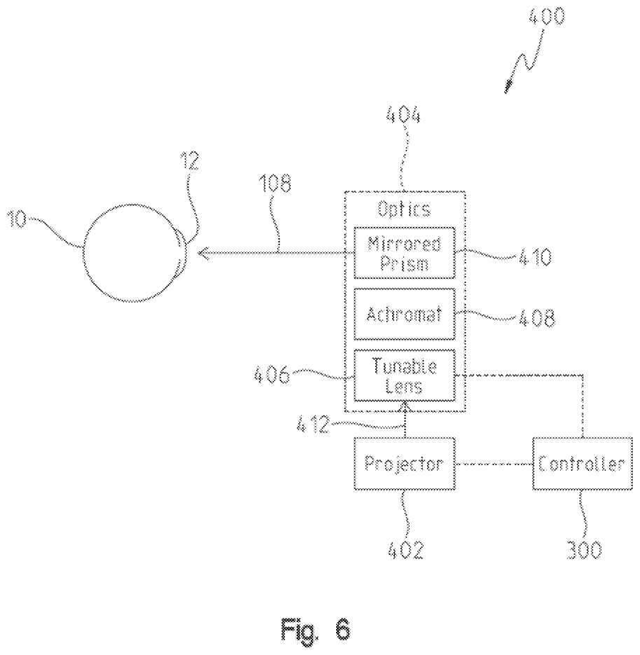

Referring to FIG. 6, an exemplary illumination system 400, as an example of illumination system 102, is shown. Illumination system 400 includes a projector 402 and an optical assembly 404. In embodiments, projector 402 is a digital light projector. An exemplary digital light projector is Model No. DLPDLCR3310EVM LightCrafter digital micromirror device (DMD) available from Texas Instruments located at 12500 TI Blvd. in Dallas, Texas 75243 United States. Another exemplary digital light projector is the LC3010 projector from Visitech/Keynote Photonics located at 1301 Central Expressway South, Suite 120 Allen, Texas 75013, United States Projector 402 is operatively coupled to controller 300 which controls the image to be displayed by projector 402. In embodiments, controller 300 is internal to projector 402. In embodiments, projector 402 communicates with controller 300 over a wired or wireless network or connection. As used herein, controller 300 encompasses the functionality of projector 402 and other controllable components of cameras 130, illumination system 102, observation system 104, optical assembly 404, optical assembly 508, and/or optical assembly 512 and controller 300 may be a single controller 300 or a collection of individual controllers that together perform the functionality of controller 300. In embodiments, illumination system support arm 103 and/or observation system support arm 105 include respective actuator systems 107, 109 (see FIG. 4), such as a motor and gearset, which are controlled by controller 300 to control an angular position of illumination system support arm 103 and/or observation system support arm 105 relative to moveable base 140 and/or each other. Exam logic 320 of controller 300, along with controlling other parameters, such as depth of focus described herein, controls the position of illumination system support arm 103 and/or observation system support arm 105 along with shapes of the slit projected at eye 10 during an examination. For example, exam logic 320 of controller 300 may simulate a conventional slit lamp exam. The positions of illumination system support arm 103 and/or observation system support arm 105 are recorded by controller 300 and associated with captured images 350 of observation system 104 for retrieval by controller 300. Additional details of systems, sensors, and characteristics to be captured with images are disclosed in U.S. Pat. No. 10,092,183, the entire disclosure of which is expressly incorporated by reference herein.

Optical assembly 404 projects the image produced by projector 402 onto eye 10. In embodiments, optical assembly 404 is adjustable to alter a focus plane of optical assembly 404 and thus focus the image produced by projector 402 at different depths of eye 10. In embodiments, the focal length of optical assembly 404 is adjustable due to the inclusion of at least one tunable lens 406 whose focal length may be altered without altering an overall position of the lens 406 relative to other components of optical assembly 404. Exemplary tunable lens 406 include electrically tunable lens available from Optotune Switzerland AG located at Bernstrasse 388, 8953 Dietikon, Switzerland. Electrically tunable lens may have their curvatures adjusted and hence their focal length by applying an electrical current to the lens. By controlling the electrical current, a controller, such as controller 300 may control the focal length of tunable lens 406 and hence the focal length of optical assembly 404. In embodiments, optical assembly 404 includes multiple tunable lens.

In embodiments, the focal length of optical assembly 404 is adjustable due to the inclusion of one or more movable lenses whose overall positions relative to other lenses may altered. In examples, the one or more movable lenses are carried by stages or other movable supports whose position may be controlled with a controller, such as controller 300, to alter the focal length of optical assembly 404. In embodiments, optical assembly 404 includes a combination of one or more movable lenses and one or more tunable lenses which optionally may be movable to alter a focal length of optical assembly 404.

In the illustrated embodiment, optical assembly 404 includes an achromat 408. Achromat 408 controls chromatic aberrations when the image projected by projector 402 includes multiple wavelengths, such as to simulate a white light slit for a slit lamp examination of eye 10. In embodiments, wherein projector 402 projects a monochromatic image achromat 408 may be omitted. In other embodiments a multi-element lens such as a camera lens with its chromatic and monochromatic aberrations as well as field curvature and distortion may be used for optical assembly 404. Exemplary lens include the model NMV-50 available from NAVITAR.

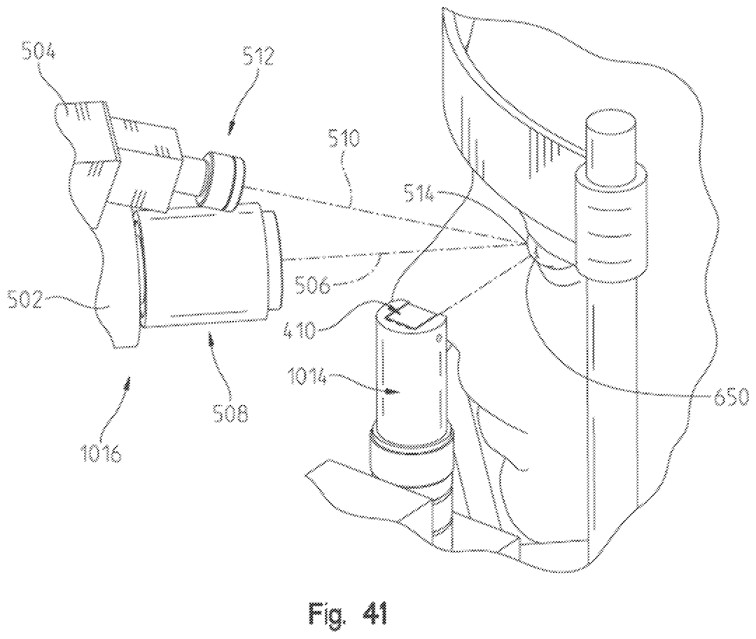

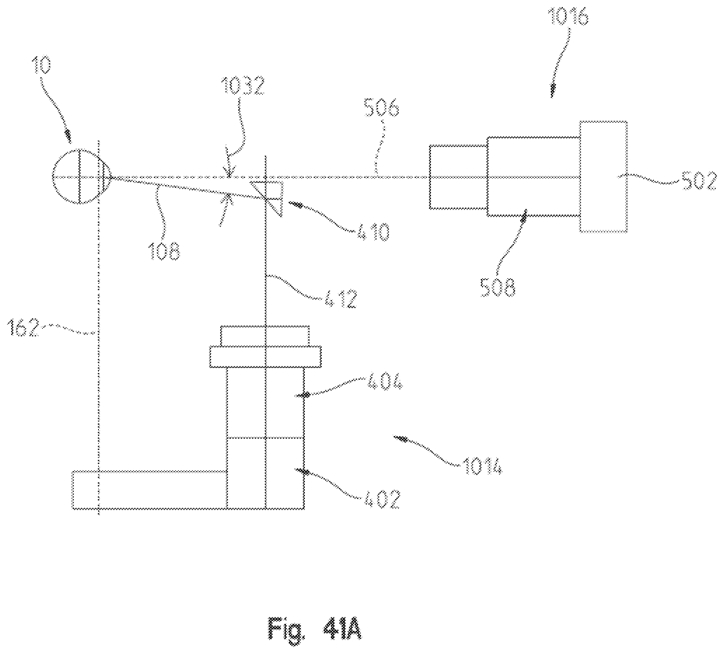

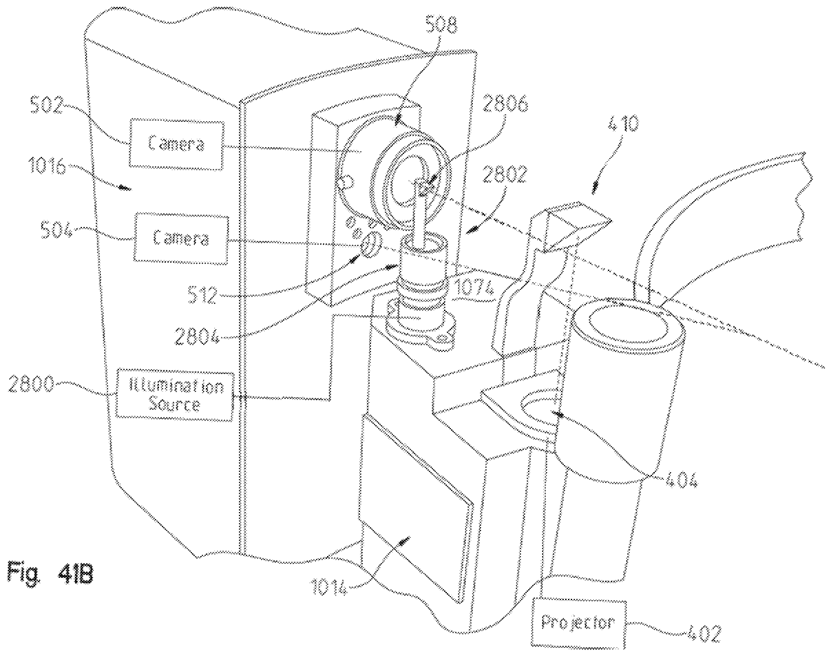

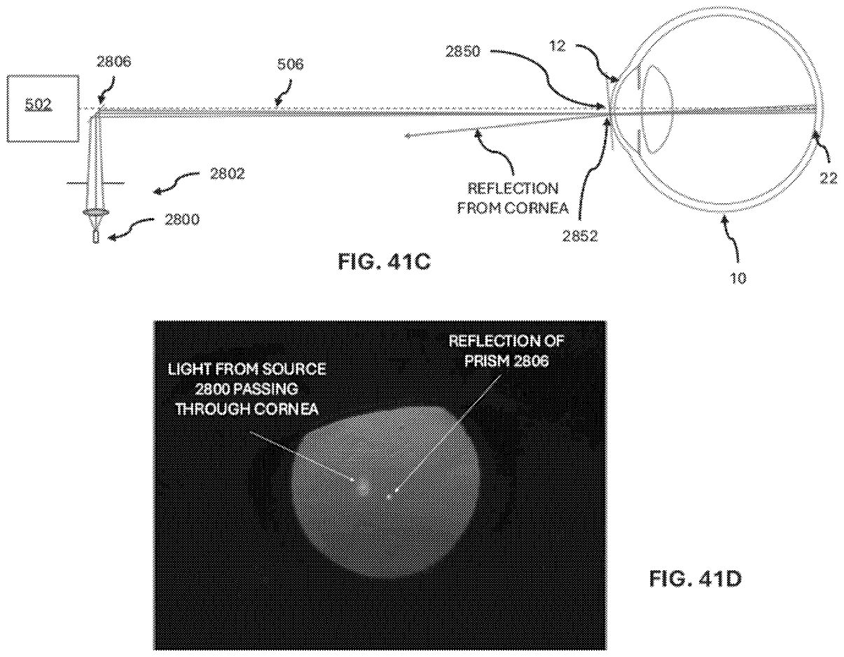

In the illustrated embodiment, optical assembly 404 includes a mirrored prism 410 which alters the optical axis that the image produced by projector 402 is projected. As shown in FIG. 6, the direction the image is projected is altered from axis 412 to optical axis 108. In embodiments wherein axis 412 is aligned with optical axis 108, mirrored prism 410 may be omitted. Further, although a mirrored prism 410 is shown, optical assembly 404 may use front surfaced mirrors or other reflective optics to alter the direction of the image projected by projector 402. An exemplary placement of mirrored prism 410 is shown in FIG. 41 for examination system 1000.

Referring to FIG. 41A, another exemplary placement of mirrored prism 410 is shown. In FIG. 41A, mirrored prism 410 is lowered relative to axis 506 and tilted such that optical axis 108 makes an angle 1032 relative to axis 506 of about three to about five degrees. Thus, mirrored prism 410 may be positioned outside of a field of view of first camera 502 while still projecting a test pattern, such as one or more vertical slits, onto eye 10. Controller 300 would still be configured to perform the various scans disclosed herein.

Referring to FIG. 41B, another exemplary embodiment of examination system 1000 is shown including mirrored prism 410 supported on platform 1074. As shown in FIG. 41B, the illustrated embodiment of examination system 1000 includes an additional illumination system 2801 including illumination source 2800 and associated optics 2802. In the illustrated embodiment, optics 2802 includes a lens system 2804 and a mirrored prism 2806. In embodiments, mirrored prism 2806 is replaced with a front surfaced mirror or other optics to direct light the output of illumination source 2800 towards eye 10. In embodiments, mirrored prism 2806 is omitted and the output of illumination source 2800 is directed towards eye 10. Exemplary illumination source 2800 include a laser diode, a fiber coupled diode laser, a light emitting diode, and other exemplary illumination sources. Illumination system may emit wavelengths to provide a white illumination, a red illumination, a near infrared illumination, and other exemplary illuminations.

As shown in the illustrated embodiment of FIG. 41B, mirrored prism 2806 may be supported by platform 1074 of illumination system 1014 while illumination source 2800 is housed in illumination system 1014 below platform 1074. As shown, mirrored prism 2806 and mirrored prism 410 are laterally offset such that in a first rotational position of illumination system 1014 mirrored prism 410 is aligned with eye 10 and first camera 502 to examine eye 10 and in a second rotational position of illumination system 1014 mirrored prism 2806 is aligned with eye 10 and first camera 502 to examine eye 10.

In embodiments, mirrored prism 2806 when aligned with eye 10 and first camera 502 is positioned off-axis from axis 506. For example, mirrored prism 2806 may be positioned so that light reflected from mirrored prism 2806 towards eye 10 makes an angle of about 1 degree to about 3 degrees with axis 506. By having optics 2802 direct light from illumination source 2800 to the cornea of eye 10 off-axis relative to axis 506 the amount of specular reflection from the surface of the cornea captured by first camera 502 is reduced. In embodiments, mirror prism 410 and mirror prism 2806 are sufficiently separated laterally so that when prism 2806 is aligned or nearly aligned with the axis 506, mirror prism 410 and its mount are effectively out of the field of view of camera 502. For example, prism 2806 and prism 410 may be laterally separated to result in a 10 degree angle being formed between their respective optical axes at the axis of rotation of assembly 1014. In embodiments, the axis of the illumination delivered through prism 2806 intersects the illumination delivered through prism 410 on the axis of rotation of assembly 1014.

The light reflected from mirrored prism 2806, in embodiments is focused on the surface of the cornea by optics 2802 (See FIG. 41C). Optics 2802 may also initially collimate the light of illumination source 2800 to expand its area past its focus. By focusing the light reflected from mirrored prism 2806 on the surface of the cornea, the light is not focused on the retina 22 of the eye 10, but rather forms a larger illuminated area on the retina 22. This larger illuminated area ensures that a portion of the light is on axis 506 and is reflected back (from the retina/fundus) along axis 506 to first camera 502 for viewing (see FIG. 41D—example image of camera 502). The light reflected back along axis 506 will generally cover an aperture defined by the pupil and thus intersect a broad portion of the cornea. With the optics 508 of camera 502 focused on the cornea, if there is a defect in a portion of the cornea from a smooth cornea surface, the reflected light at that location will be angled relative to axis 506 upon exiting the cornea resulting in a dark region on the image captured by first camera 502. In embodiments, optics 508 of camera 502 is focused on deeper layers such as the crystalline lens, the intraocular lens or the capsular bag to reveal irregularities/defects in these layers. In embodiments, the cross-sectional area of optics 2802 is substantially smaller than the aperture area of optics 508 so while optics 2802 is in front of optics 508 (aligned or nearly aligned with axis 506) a substantial fraction of the aperture of optics 508 is unobstructed and can capture light returning from the eye 10. In embodiments, optical axis 506 intersects with the apex of the cornea (location 2850 in FIG. 41C) and light from optics 2802 is focused on an area of the cornea different from the apex (location 2852 in FIG. 41C), such as about 1 millimeter lower (see FIG. 41C), so that specular reflection from the cornea is not propagated along axis 506.

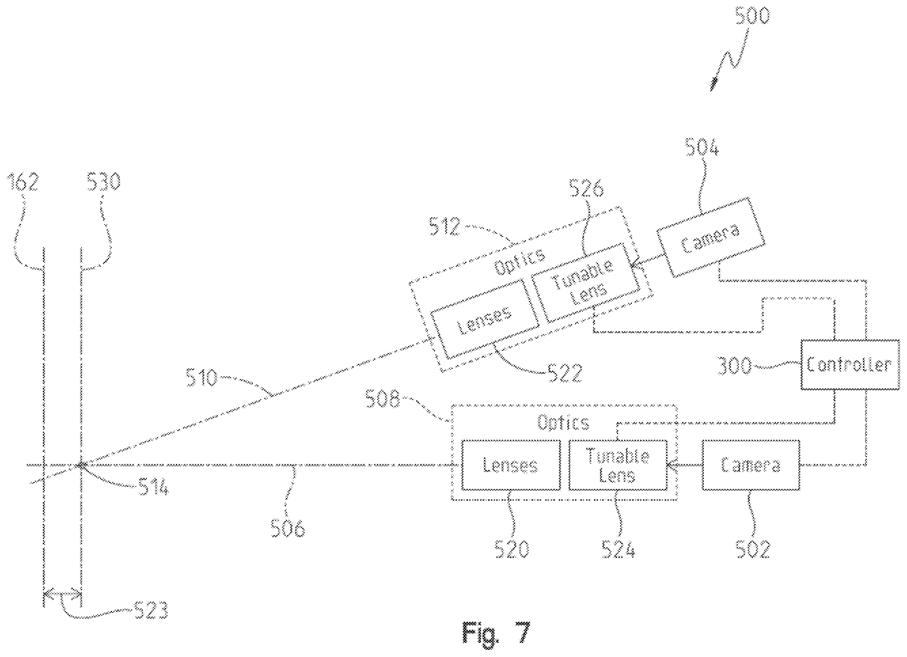

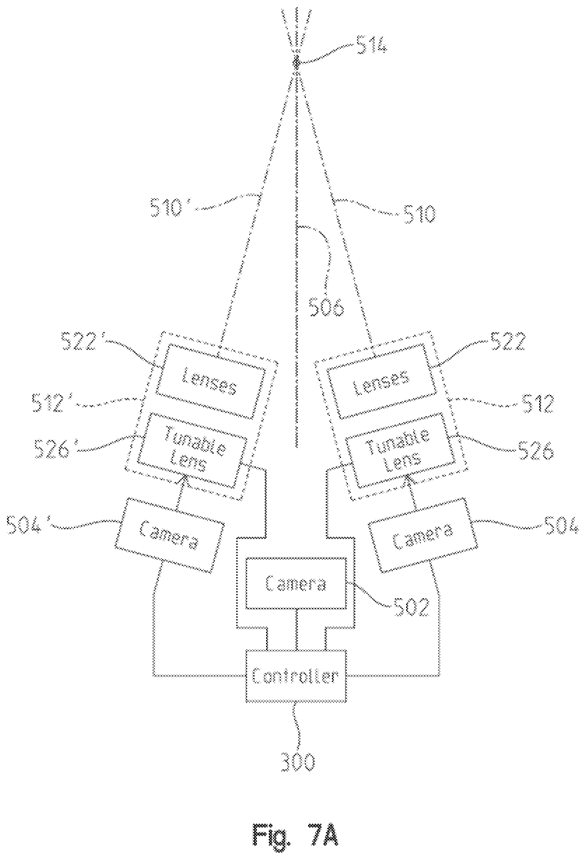

Referring to FIG. 7, an exemplary observation system 500, as an example of observation system 104, is shown. Observation system 500 includes a plurality of cameras, illustratively two cameras, which are directed toward a position of eye 10. A first camera 502 is directed along axis 506, which may be aligned with optical axis 112 (see FIG. 2), and includes an associated optical assembly 508. A second camera 504 is directed along axis 510 and includes an associated optical assembly 512. Axis 510 is angled relative to axis 506 and intersects axis 506 at location 514. In embodiments, location 514 is within a focal range of the first camera 502 and associated optics 508 and the second camera 504 and associated optics 512. In embodiments, axis 510 is coaligned with axis 506. In embodiments, axis 510 does not intersect axis 506, but rather axis 510 and axis 506 are skew. Although a single camera 504 is illustrated, in embodiments, multiple cameras 504 are provided. For example, a plurality of cameras 540 may be used to capture images to provide a stereo view of eye 10 on display 382 or stored in memory 312 for later retrieval.

In the illustrated embodiment, axis 510 is angled downwards towards axis 506 and axis 506 is horizontal. In embodiments, axis 510 is angled upward towards axis 506 and axis 506 is horizontal. In embodiments, axis 510 is horizontal and axis 506 is angled upwards or downwards towards axis 510 depending on the positioning of camera 502 and optical assembly 508. In embodiments, both axis 506 and axis 510 are angled relative to horizontal. Although shown being vertically aligned, axis 510 may be rotated about axis 506 so that axis 510 and axis 506 are horizontally aligned or aligned along a plane passing through axis 506 and angled relative to vertical.

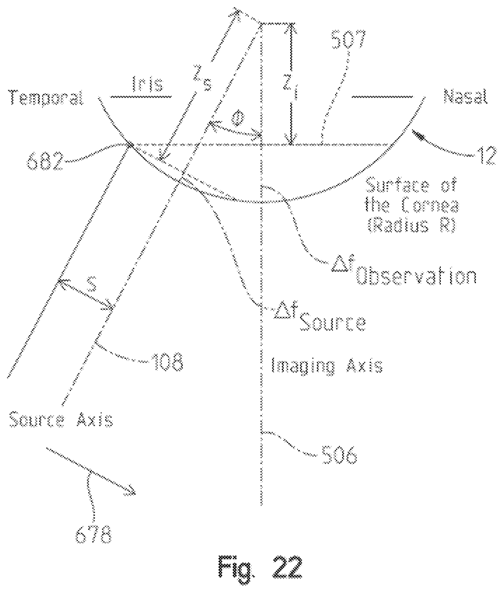

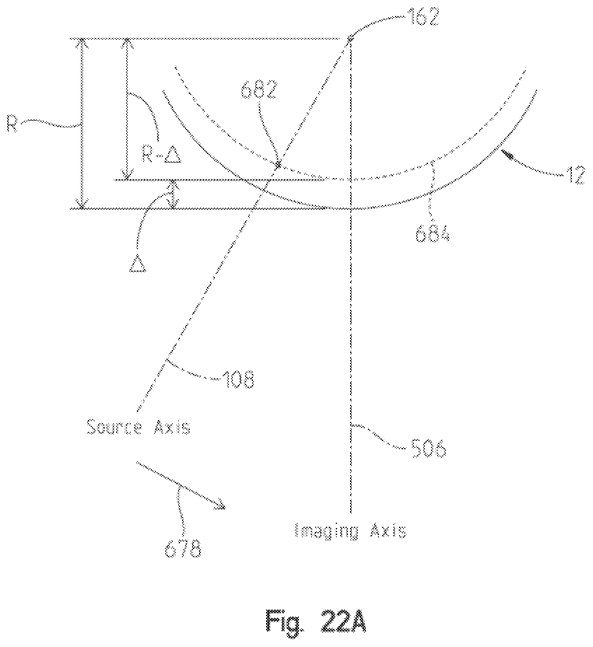

Each of optical assembly 508 and optical assembly 512 focus light reflected by eye 10, diffuse light, into the respective one of first camera 502 and second camera 504. In the illustrated embodiment, each of optical assembly 508 and optical assembly 512 includes one or more lenses 520, 522 and one or more tunable lenses 524, 526. In embodiments, one or both of optical assembly 508 and optical assembly 512 may also include reflective optics, which optionally may introduce power into the respective optical assembly 508, 512. Further, in embodiments, one or both of optical assembly 508 and optical assembly 512 may omit one of lenses 520, 522 or tunable lenses 524, 526. Regardless of the make-up of the respective optical assembly 508 and optical assembly 512, each of optical assembly 508 and optical assembly 512 is adjustable to alter a focal plane of the respective first camera 502 and second camera 504. In embodiments, at least one plane 507 (see FIG. 22) to be imaged by observation system 500 is normal to the optical axis 506 of the observation system 500. Observation system 500 is focused to image the plane including point 682. As discussed herein, lenses, such as lenses 520, 522, may be provided on movable supports whose positions may be adjusted to alter a focal length of the respective optical assembly 508 and optical assembly 512 and tunable lenses, such as tunable lenses 524 and 526, may have a focal length of the lens altered by altering an electrical current applied to the respective tunable lenses 524 and tunable lenses 526.

In embodiments, first camera 502 and second camera 504 are each digital cameras having detector arrays comprised of pixels which record the light reflected from eye 10 and focused by the respective optical assembly 508 and optical assembly 512 on the detector array of the respective first camera 502 and second camera 504. The detector arrays may include multiple pixel types, such as a red wavelength sensitive detection pixel, a green wavelength sensitive detection pixel, and a blue wavelength sensitive detection pixel. In embodiments, one or both of first camera 502 and second camera 504 are light field cameras which record light field data to allow through digital processing to further alter a focus of an image. Exemplary light field cameras include plenoptic cameras.

As shown in FIG. 7, a vertical axis 530 passing through location 514 is offset from axis 162 by a first distance 523. In embodiments, first distance 523 is about 7.7 millimeters (mm). In embodiments, first distance 523 is between about 5 mm to about 10 mm. In embodiments, vertical axis 530 is coaligned with axis 162 and first distance 522 is zero. In embodiments, vertical axis 530 is set to equal a radius of curvature of cornea 12 of eye 10. By having axis 162 behind vertical axis 530, axis 162 may be placed approximately at the center of curvature for cornea 12 of eye 10 and location 514 on the surface of cornea 12 of eye 10. Thus, as observation system 104 is rotated about axis 162, each of first camera 502 and second camera 504 may remain focused on cornea 12 of eye 10 assuming a spherical model for cornea 12 of eye 10. Similarly, as illumination system 102 is rotated about axis 162, the image projected by illumination system 102 may remain focused on cornea 12 of eye 10 assuming a spherical model for cornea 12 of eye 10. In embodiments, adjustments to the focal position of illumination system 102 and observation system 104 may be made by controller 300 to move location 514 at different depths within eye 10 or to follow an aspheric model of cornea 12 of eye 10, as described herein.

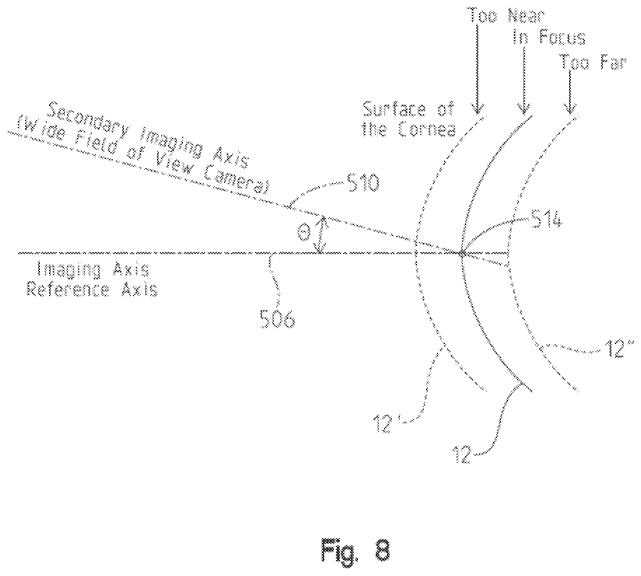

Turning to FIG. 8, location 514 is shown positioned on the surface of cornea 12 of eye 10. Cornea 12 is also shown at a position too near to first camera 502 and second camera 504, as represented by cornea 12′, and at a position too far from first camera 502 and second camera 504, as represented by cornea 12″. If the cornea 12 is either too near or too far moveable base 140 may be adjusted to move location 514 to the surface of cornea 12. In embodiments, moveable base 140 may be moved manually. In embodiments, moveable base 140 is moved by controller 300 automatically. Alternatively to moving the movable base 140, one or both of optics 508 and optics 512 may be adjusted by controller 300 to move the location 514. In embodiments, both moveable base 140 and at least one of optics 508 and optics 512 are adjusted to move the location 514, such as by controller 300. Controller 300 may also adjust the focus of illumination system 102 and the lateral position of the light projected by illumination system 102. The lateral position of the light projected by illumination system 102 may be adjusted by changing the image projected by projector 402, such as changing which pixels correspond to the slit portion of the image instead of the background portion of the image (see FIG. 26).

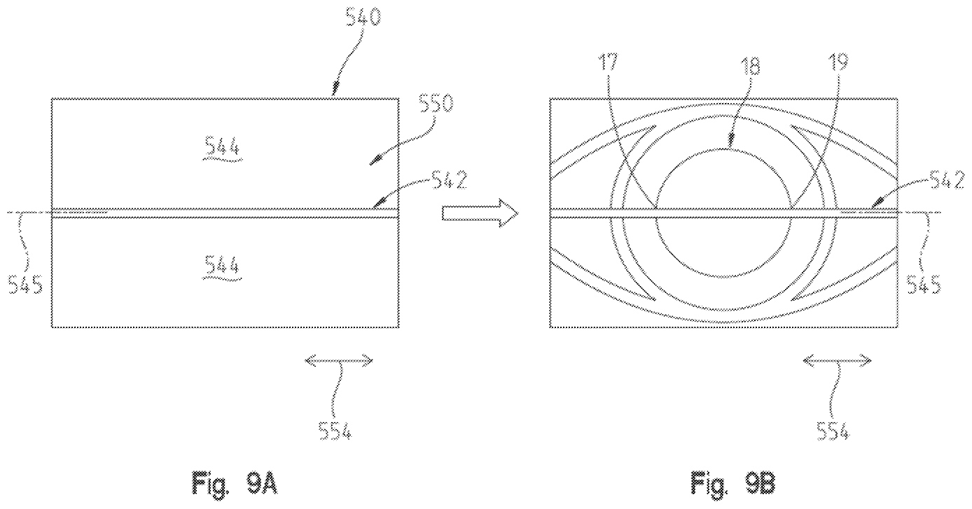

In embodiments, source 200 of illumination system 102 may be used to assist in positioning location 514 at the surface of cornea 12. For example, illumination system 102 may project a test pattern 550 (see FIG. 9A) on eye 10 (see FIG. 9B). Referring to FIG. 9A, an image 540 of an exemplary test pattern is shown having a bright central horizontal slit 542 and diffuse lower light regions 544. In embodiments, image 540 is produced by projector 402 and projected onto eye 10 as shown in FIG. 9B (image observed by camera 502). As shown in FIGS. 9A and 9B, the orientation of slit 542 is the same. In embodiments, optical assembly 404 flips or otherwise alters the orientation of an image produced by projector 402 as it passes through optical assembly 404 and is projected onto eye 10. For simplicity, the images produced by projector 402 provided herein are shown to have the intended orientation as projected on eye 10.

As shown in FIG. 9B, horizontal slit 542 is vertically aligned with the center of pupil 18 of eye 10. If horizontal slit 542 is not vertically aligned with the center of pupil 18 of eye 10, patient support 144 may be adjusted up in direction 154 or down in direction 156 to center horizontal slit 542 on pupil 18 of eye 10. In embodiments, controller 300 automatically adjusts a height of patient support 144 to vertically align horizontal slit 542 with the center of pupil 18 of eye 10. For example, controller 300 may analyze the intensity values of the pixels recorded by first camera 502 to determine a left edge 17 and a right edge 19 of pupil 18 and move patient support 144 up and down to determine a height position whereat a separation between left edge 17 and right edge 19 is at a maximum. The determined height position corresponds to horizontal slit 542 being vertically aligned with the center of pupil 18 of eye 10. In embodiments, the height of patient support 144 is manually adjusted by the person performing the test on the patient to vertically align horizontal slit 542 with the center of pupil 18 of eye 10.

Once horizontal slit 542 is vertically aligned with the center of pupil 18 of eye 10 (as shown in FIG. 9B), controller 300 may execute logic to align examination system 100 relative to eye 10 in directions 150, 152. In the illustrated embodiment, controller 300 executes logic to position location 514 on the surface of cornea 12 of eye 10 at the apex of the cornea 12 of the eye 10. In other embodiments, examination system 100 may be aligned relative to eye 10 in directions 150, 152 by focusing on other structures of eye 10, such as the iris structure, the location of the limbus, and/or the pupillary margin and using population averages of the anterior chamber depth from ocular tomography or laser interferometry approximate the location of the corneal apex and position direction 154 at this approximated location. Additionally, other techniques may be used to align examination system 100 relative to eye 10 including range distance finders, LiDAR, a calculated depth map from a Lightfield camera or stereo cameras. In embodiments, contrast detection autofocus, phase detection autofocus or dual pixel autofocus are used to maintain focus on the cornea, iris, lens, and/or other eye structures.

Referring to FIG. 7A, a third camera 504′ and associated optics 512′ are shown positioned symmetrically to second camera 504 with respect to the axis 506 of the first camera 502. Second camera 504 and third camera 504′ are both positioned in the same plane and are angled relative to first camera 502 in the same manner as second camera 504 in FIG. 7. Second camera 504 and third camera 504′ form a stereo pair. In embodiments, second camera 504 and third camera 504′ are used to adjust the position of location 514 relative to cornea 12. In embodiments controller 300 maintains actively the position of 514 on the surface of the cornea 12 based on second camera 504 and third camera 504′ by moving movable base 140 while leaving the first camera 502 available for high resolution imaging. Additionally, other techniques may be used to align examination system 100 relative to eye 10 including range distance finders, LiDAR, a calculated depth map from a light-field camera or stereo cameras. In embodiments, contrast detection autofocus, phase detection autofocus or dual pixel autofocus are used to maintain focus on the cornea, iris, lens, and/or other eye structures.

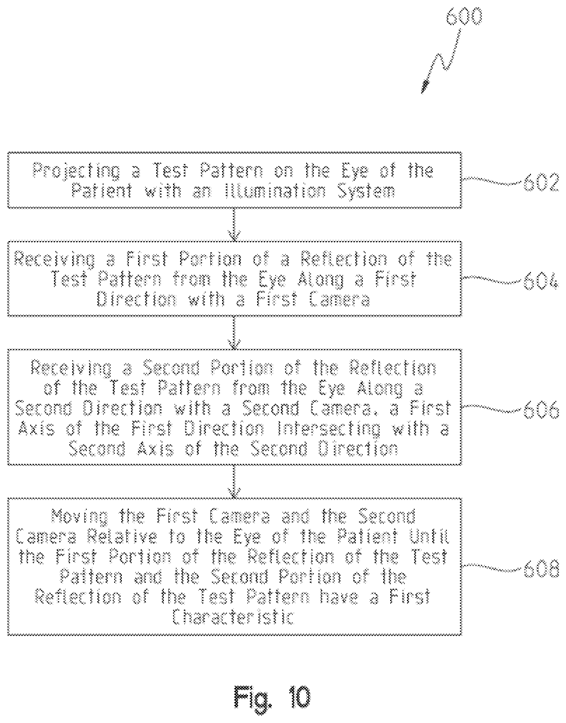

Turning to FIG. 10, an exemplary processing sequence 600 of exam logic 320 of controller 300 is shown for aligning the cameras 130 of examination system 100 to the cornea 12 of eye 10. In embodiments, a technician may use examination system 100 to carry out the steps of processing sequence 600 in a manual operation.

Referring to FIG. 10, a test pattern is projected onto eye 10 of the patient with an illumination system, such as illumination system 102, as represented by block 602. In embodiments, the test pattern projected onto the eye 10 of the patient with the illumination system 102 is programmed into a digital light projector, such as projector 402, to display the test pattern and project it onto eye 10. In an example, the test pattern is a horizontal line.

Returning to FIG. 10, a first portion of a reflection of the test pattern from the eye is received along a first direction with a first camera, such as first camera 502, as represented by block 604. A second portion of the reflection of the test pattern from the eye is received along a second direction with a second camera, such as second camera 504, as represented by block 606. A first axis 506 of the first direction intersecting with a second axis 510 of the second direction. The first camera 502 and the second camera 504 are moved relative to the eye 10 of the patient until the first portion of the reflection of the test pattern and the second portion of the reflection of the test pattern have a first characteristic, as represented by block 608.

In embodiments, the first camera 502 and the second camera 504 are moved relative to the eye 10 of the patient by actuating a positioning system of the imaging system with a movement command from controller 300 based on the first portion of the reflection of the test pattern and the second portion of the reflection of the test pattern having a characteristic different from the first characteristic. Alternatively, the first camera 502 and the second camera 504 are moved relative to the eye 10 of the patient by receiving a manual input to a support, such as moveable base 140, of at least one of the first camera 502 and the second camera 504 to reposition the at least one of the first camera 502 and the second camera 504 relative to the eye 10 of the patient. Alternatively, as mentioned herein, the characteristics of one or more of optics 508 and optics 512 may be adjusted instead of moving moveable base 140 or in addition to moving moveable base 140.

In embodiments, processing sequence 600 further includes displaying the first portion of the reflection of the test pattern and the second portion of the reflection of the test pattern on a display. This provides a visual cue to the operator of examination system 100 of the alignment of examination system 100 to cornea 12 of the patient. In embodiments, each of illumination system 102, first camera 502, and second camera 504 are supported on a movable base 140 such that illumination system 102, first camera 502, and second camera 504 move as a unit relative to the eye 10 of the patient.

In embodiments, the test pattern includes a first elongated feature having a longitudinal axis in a third direction, the third direction being normal to a plane defined by the first direction along which the first portion of a reflection of the test pattern from the eye is received with first camera 502 and the second direction along which the second portion of a reflection of the test pattern from the eye is received with second camera 504. The first portion of the reflection of the test pattern may include a first portion of the elongated feature of the test pattern and the second portion of the reflection of the test pattern may include a second portion of the elongated feature of the test pattern. In embodiments, the first portion of the elongated feature of the test pattern and the second portion of the elongated feature of the test pattern are aligned along the third direction in the first characteristic indicating that the first camera 502 and the second camera 504 are aligned to the eye 10 of the patient by having location 514 aligned with the surface of cornea 12. When location 514 is not aligned with the surface of cornea 12, the first portion of the elongated feature of the test pattern and the second portion of the elongated feature of the test pattern are offset along the third direction, such as vertically offset when the third direction is horizontal, in a second characteristic indicating that at least one of the first camera 502 and the second camera 504 are misaligned to the eye 10 of the patient.

Referring to FIGS. 9A, 9B, 11A, 11B, and 12-14, an exemplary test pattern 550 (see FIG. 9A) is projected on eye 10 (see FIG. 9B). Referring to FIG. 9A, an image 540 of an exemplary test pattern 550 is shown having a bright central horizontal slit 542 and diffuse lower light regions 544. In embodiments, image 540 is produced by projector 402 and projected onto eye 10 as shown in FIG. 9B (image observed by camera 502). As shown in FIGS. 9A and 9B, the orientation of slit 542 is the same. In embodiments, optical assembly 404 flips or otherwise alters the orientation of an image produced by projector 402 as it passes through optical assembly 404 and is projected onto eye 10. For simplicity, the images produced by projector 402 provided herein are shown to have the intended orientation as projected on eye 10.

Horizontal slit 542 is an example elongated feature of test pattern 550. In embodiments, test pattern 550 includes multiple elongated features and/or additional feature shapes. The elongated features may be oriented in a horizontal direction, a vertical direction, a direction angled relative to horizontal, or combinations thereof. Test pattern 550 may include an elongated feature, such as horizontal slit 542, having a longitudinal axis 545 in a third direction 554. Third direction 554 is normal to a plane defined by the first direction of axis 506 and the second direction of axis 510. Stated another way third direction 530 is contained in a plane normal to the view of FIG. 7.

Main camera 502 receives a first portion of the reflection of the test pattern 550 (image 560 in FIG. 11A) which includes a first portion 546 of the elongated feature, horizontal slit 542, of the test pattern 550. Overview camera 504 receives a second portion of the reflection of the test pattern 550 (image 562 in FIG. 11B) which includes a second portion 548 of the elongated feature, horizontal slit 542, of the test pattern 550. First portion 546 of the elongated feature, horizontal slit 542, of the test pattern 550 and second portion 548 of the elongated feature, horizontal slit 542, of the test pattern 550 are included in sub-image 564 (box bounded by A) in image 560 and in sub-image 566 (box bounded by B) in image 562, respectively. Controller 300 based on the magnification of first camera 502 and second camera 504 scales sub-image 566 of image 562 to be the same vertical height as sub-image 564 of image 560.

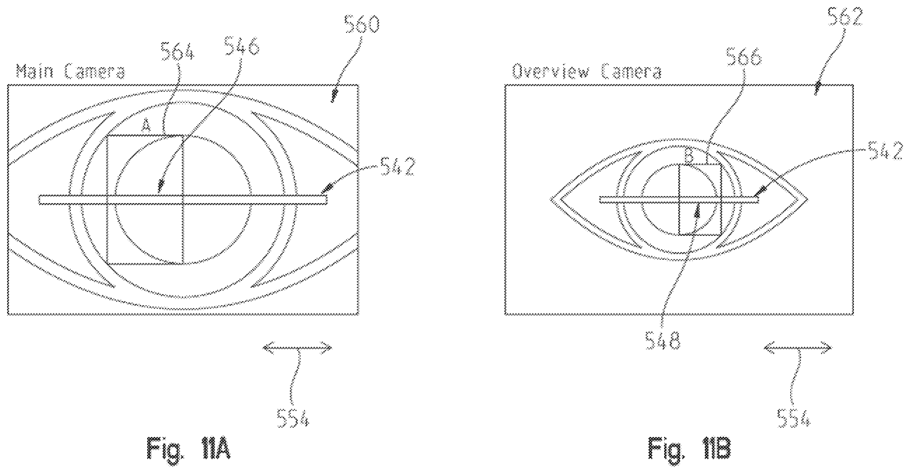

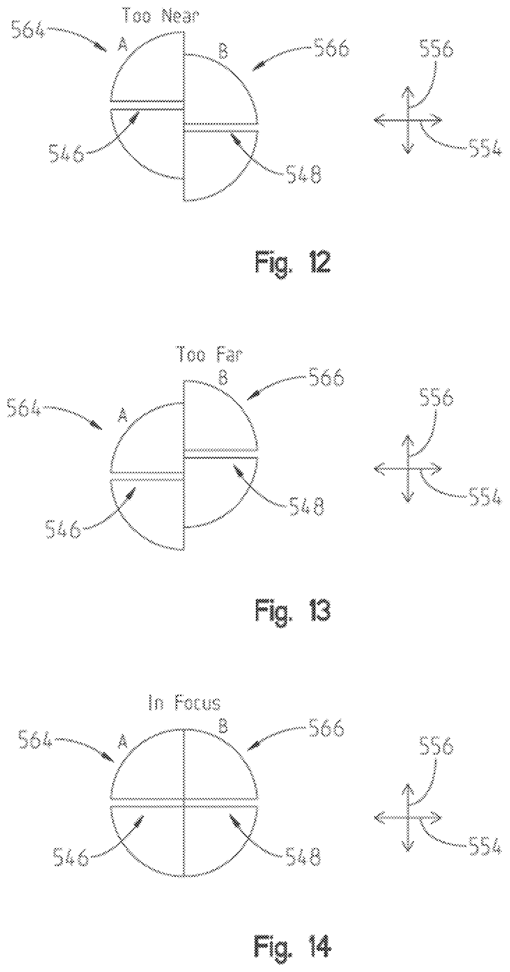

By comparing first portion 564 of the elongated feature, horizontal slit 542, of the test pattern 550 and the second portion 566 and the elongated feature, horizontal slit 542, of the test pattern 550 a determination of an alignment of first camera 502 and second camera 504 to eye 10 may be made. Referring to FIG. 14, first portion 546 and second portion 548 are vertically aligned in a direction 556 which is normal to direction 554 which is a first characteristic that indicates that location 514 (see FIG. 8) is positioned on the surface of cornea 12 of eye 10. Referring to FIG. 12, first portion 546 and second portion 548 are vertically offset in direction 556 with first portion 546 higher than second portion 548 indicating location 514 is behind the surface of the cornea (cornea in position 12′ relative to location 514 in FIG. 8). Referring to FIG. 13, first portion 546 and second portion 548 are vertically offset in direction 556 with second portion 548 higher than first portion 546 indicating location 514 is in front of the surface of the cornea (cornea in position 12″ relative to location 514 in FIG. 8).

In embodiments, sub-image 564 and sub-image 566 are displayed on a display 382 of examination system 100. Display 382 may be part of first camera 502 or second camera 504 or a separate display. An operator of examination system 100 may manually move moveable base 140 in one of direction 150 or direction 152 to adjust the position of location 514 relative to cornea 12. For example, by moving moveable base 140 in direction 152, second portion 548 in FIG. 12 may be raised to align with first portion 546 resulting in the image of FIG. 14 when location 514 is located on the surface of cornea 12. In another example, by moving moveable base 140 in direction 150, second portion 548 in FIG. 12 may be lowered to align with first portion 546 resulting in the image of FIG. 14 when location 514 is located on the surface of cornea 12.

In embodiments, moveable base 140 includes a positioning system 180 which includes one or more powered actuators to move moveable base 140 relative to patient support 144. Controller 300 is operatively coupled to positioning system 180 to move moveable base 140 relative to patient support 144 based on a detected position of first portion 546 and second portion 548 by controller 300. In embodiments, controller 300 executes a processing sequence 630 of exam logic 320 to align examination system 100 to eye 10. In embodiments, controller 300 aligns examination system 100 to an apex (see point 514 in FIG. 8) of cornea 12 of the eye 10. Turning to FIG. 15, an image, such as image 540 of FIG. 9A, is projected on surface of eye 10, as represented by block 632. A first portion of the image is monitored with a first camera, such as sub-image 564 captured by first camera 502 in FIG. 11A, as represented by block 634. A second portion of the image is monitored with a second camera, such as sub-image 566 captured by second camera 504 in FIG. 11B, as represented by block 636. Controller 300 determines if the portions of the image satisfy a first criteria, as represented by block 638. In the embodiment of FIGS. 11-14 the first criteria is whether first portion 546 and second portion 548 are vertically aligned. In other embodiments, other criteria may be used such as whether features of the two images overlap or have a predetermined positional relationship to each other. If the first criteria is satisfied, processing sequence 630 exits as represented by block 640. In embodiments, controller 300 provides an indication of alignment to an operator of examination system 100 with one or more of output devices 380. If the first criteria is not satisfied, controller 300 moves moveable base 140 relative to patient support 144 with positioning system 180, as represented by block 642. In the embodiment of FIGS. 11-14, when second portion 548 is lower than first portion 546, controller 300 provides a movement command to positioning system 180 to move moveable base 140 in direction 152 until second portion 548 is aligned with first portion 546. When second portion 548 is higher than first portion 546, controller 300 provides a movement command to positioning system 180 to move moveable base 140 in direction 150 until second portion 548 is aligned with first portion 546. When second portion 548 is aligned with first portion 546, examination system 100 is aligned with an apex (see point 514 in FIG. 8) of cornea 12 of the eye 10.

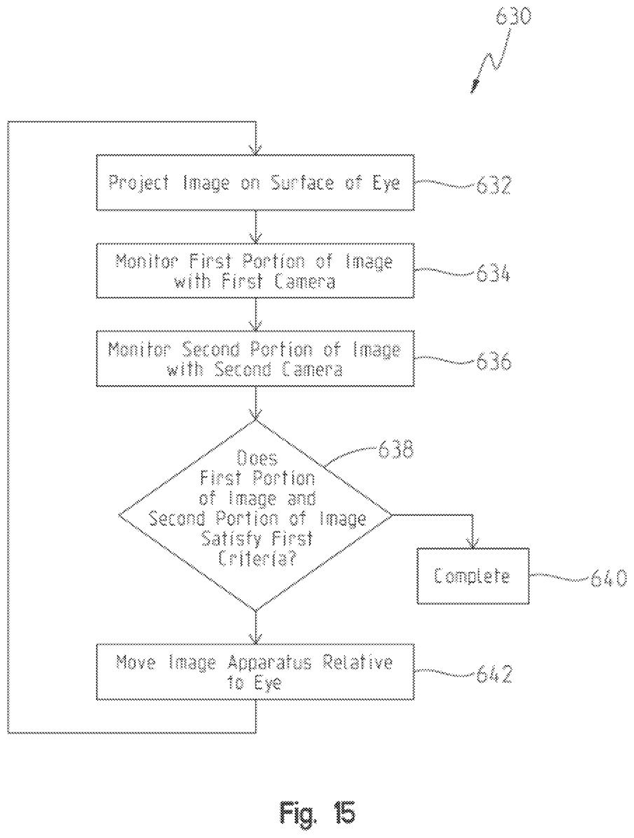

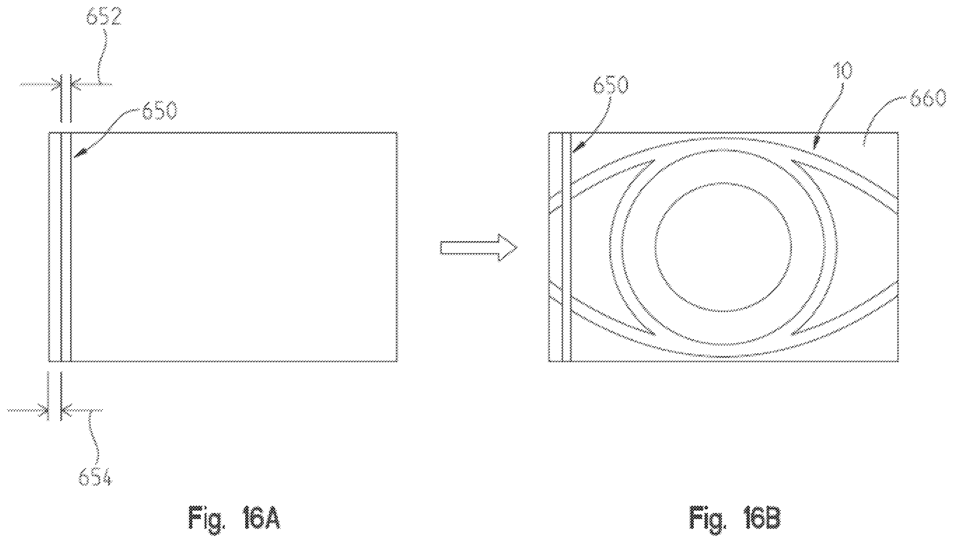

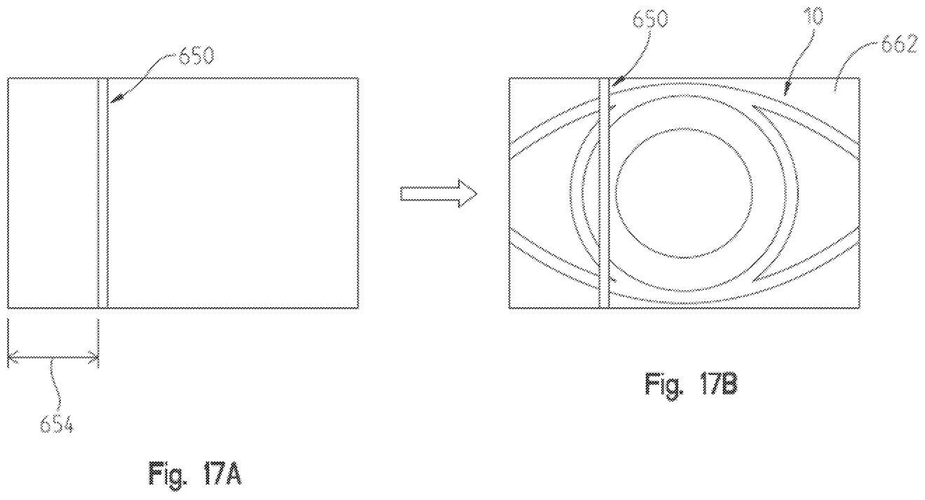

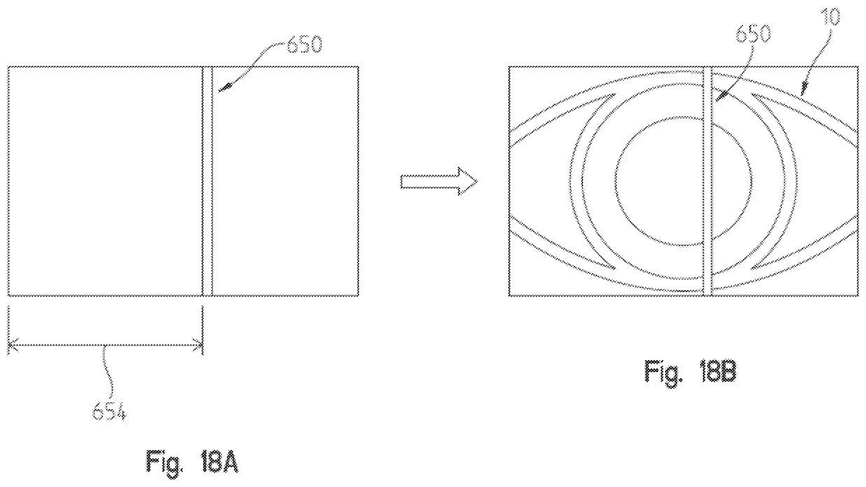

Illumination system 102 with projector 402 is able to generate a test pattern which is projected by optical assembly 404 onto the eye. Exemplary test patterns, include a virtual vertical slit 650 (see FIG. 16A with vertical slit 650 at a first transverse position) which is projected by optical assembly 404 onto cornea 12 of eye 10 and stored as an image 660 by camera 502 (see FIG. 16B) and camera 504 and viewable on display 382. Slit 650 may have a width 652 which may be widened or narrowed by controller 300 through the software of projector 402. In embodiments, slit 650 has a width in the range of about 0.1 millimeters to about 15 millimeters. In embodiments, slit 650 has a width in the range of about 0.25 millimeters to about 8 millimeters. Additionally, a horizontal offset 654 of vertical slit 650 from a left edge of the image projected by projector 402 may be altered by controller 300 through the software of projector 402. Referring to FIG. 17A, vertical slit 650 has moved further to the right in the projected image to a second transverse position offset from the first transverse position of FIG. 16A and is stored as image 662 (see FIG. 17B) by first camera 502. Referring to FIG. 18A, vertical slit 650 has moved further to the right than in FIG. 17A in the projected image to a third transverse position offset from the first transverse position of FIG. 16A and the second transverse position of FIG. 17A and is stored as image 664 (see FIG. 18B) by first camera 502. The width 652 and horizontal offset 654 of vertical slit 650 may be controlled by controller 300 in embodiments or by an operator of examination system 100 through one or more input devices 360 of examination system 100.

The projected test pattern, such as vertical slit 650, may have different color values in different images. For example, projector 402 may project a vertical slit 650 with a first color value in a first image and with a second color value in a second image, the second color value being different from the first color value. Exemplary color values include a red color, a blue color, a white color, a red-free color, and other suitable colors. Also, the projected test pattern, such as vertical slit 650, may have different color values in different test scenarios. For example, projector 402 may project a vertical slit 650 with a first color value in a plurality of transverse positions during one type of examination and with a second color value in a plurality of positions for a different examination. Exemplary color values include a red color, a blue color, a white color, a red-free color, and other suitable colors. A blue color may be used for fluorescein mapping. A white color or red-free color for corneal imaging. A red color may be used for retro-illumination images.

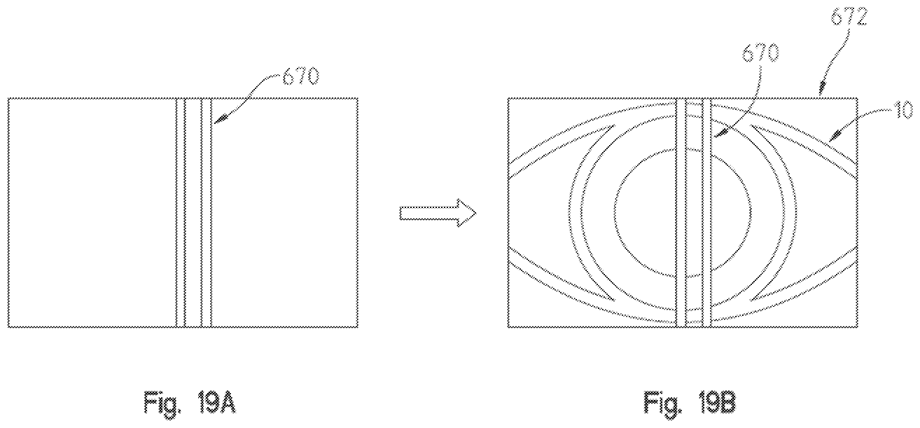

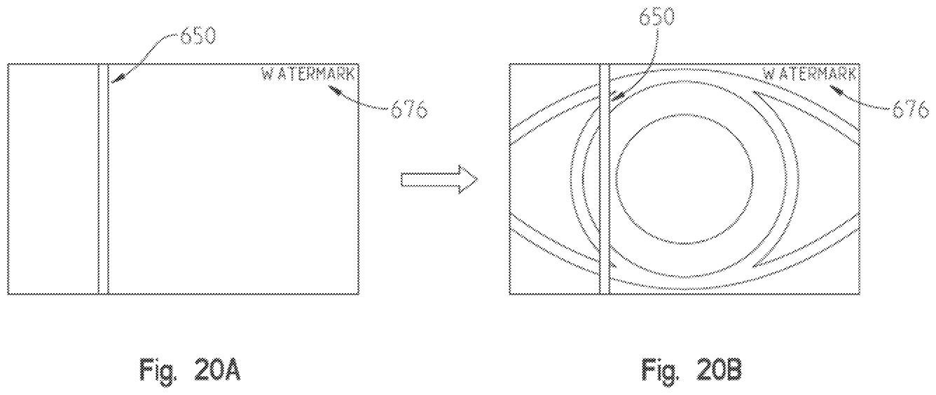

Along with altering width 652 and/or horizontal offset 654 of vertical slit 650, different features may be projected by projector 402 onto eye 10. In FIG. 19A a double slit 670 is projected by projector 402 and stored as an image 672 by first camera 502 (see FIG. 19B). Additionally, a watermark 676 may be included in the image projected by projector 402 onto eye 10 and imaged by first camera 502. Further, textual data may be included, such as a time stamp, patient name, indications of nasal and temporal sides of image, and other suitable information. Although a double slit 670 is shown in FIG. 19A, in embodiments, three or more slits may be shown simultaneously.

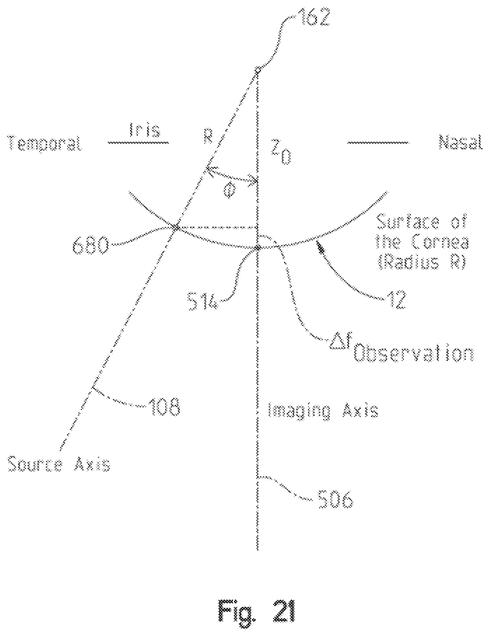

Referring to FIG. 21, an outer surface of cornea 12 of eye 10 is represented as a sphere of radius (R) equal to the difference between axis 162 and location 514. Axis 162 is the common rotation axis of illumination system 102 and observation system 104. Location 514 is the intersection of axis 506 of first camera 502 and optical assembly 508 and axis 510 of second camera 504 and optical assembly 512. Both illumination system 102 (axis 108 shown) and observation system 104 should be focused at the same depth to properly form (for illumination system 102) and image (for observation system 104) a slit 650 (see FIG. 16A) projected onto 12.

In FIG. 21, virtual vertical slit 650 is positioned along optical axis 108 of illumination system 102 and focused on cornea 12 as represented by position 680. In embodiments, controller 300 has a stored default focal length for illumination system 102 to focus virtual vertical slit 650 on cornea 12 when illumination system 102 is rotated by an angle o relative to axis 506 of observation system 104. Since illumination system 102 is focused at position 680, camera 502 and camera 504 should also be focused at a depth corresponding to position 680. As shown in FIG. 21, angle φ and the estimated value for R are known by controller 300. Therefore, controller 300 may determine the change in focus (Δf_{OBSERVATION}) needed to focus observation system 104 at a depth corresponding to position 680 according to equation 1.

Δf_{OBSERVATION}=R(1−cosφ) (1)

Referring to FIG. 22, same arrangement as FIG. 21, except that virtual vertical slit 650 is now offset from optical axis 108 by a distance S, as represented by position 682. Both the focus of the illumination system 102 and the observation system 104 need to be altered to focus at position 682. As shown in FIG. 22, angle φ, the offset distance S, and the estimated value for R are known by controller 300. Therefore, controller 300 may determine the change in focus (Δf_{SOURCE}) needed to focus illumination system 102 at a depth corresponding to position 682 according to equation 2.

Δf_{SOURCE}=R−zₛ wherein zₛ=\sqrt{R²−S²} (2)

Controller 300 may also determine the change in focus (Δf_{OBSERVATION}) needed to focus observation system 104 at a depth corresponding to position 682 according to equation 3.

Δf_{OBSERVATION}=R−zᵢ wherein zᵢ=\sqrt{R²−p²} and p=S cos(φ)+zₛsin(φ) (3)

Controller 300 may make adjustments to the focal lengths of illumination system 102 and observation system 104 as virtual vertical slit 650 is moved across cornea 12 to maintain both virtual vertical slit 650 and the image of virtual vertical slit 650 observed by observation system 104 in focus. In embodiments, virtual vertical slit 650 is moved across cornea 12 in transverse direction 678 to obtain multiple images with observation system 104 of virtual vertical slit 650 focused on cornea 12 at spaced apart locations. The changes in focus of illumination system 102 and observation system 104 may be made by controller 300 adjusting one or more characteristics of optical assembly 404 of illumination system 102 for (Δf_{SOURCE}) and one or more characteristics of optical assembly 508 and/or optical assembly 512 of observation system 104 for (Δf_{OBSERVATION}). In embodiments, since each of adjustments to illumination system 102 and observation system 104 may be made by adjusting components internal to each of illumination system 102 and observation system 104, the relative position of illumination system 102 and observation system 104 to cornea 12 does not need to change. Stated another way, each of illumination system 102 and observation system 104 may remain stationary relative to cornea 12 as virtual vertical slit 650 is moved across cornea 12.

Although in each of FIGS. 21 and 22, the surface of cornea 12 is represented by a sphere of radius R, controller 300 may use other shapes to correspond to the shape of cornea 12, such as aspheric curves, polynomial curves, and other shapes. The example provided in FIGS. 21 and 22 is a non-limiting example. In other embodiments, a shape of cornea 12 is determined by controller 300 by projecting a test pattern, such as concentric rings similar to those produced by a keratoscope, onto cornea 12 and measuring changes in the test pattern imaged by examination system 100. In other embodiments, corneal curvature measurements obtained using scanning slit technology, Scheimpflug-based imaging or anterior segment ocular coherence tomography may be used to provide an input to controller 300 for determining the shape of cornea 12.

In embodiments, controller 300 may also intentionally focus virtual vertical slit 650 at a location offset from cornea 12, such as behind cornea 12 deeper into eye 10. In a similar manner, controller 300 would then also focus observation system 104 at a depth corresponding to the offset from cornea 12. As shown in FIG. 22A, position 682 is positioned behind cornea 12, on a construction circle 684 of radius (R-Δ) wherein R is the radius of cornea 12 and Δ is the offset to construction circle 684. Each of cornea 12 and construction circle 684 have their respective centers of curvature aligned with axis 162.

Using the shape of construction circle 684, controller 300 may once again move virtual vertical slit 650 across eye 10 in transverse direction 678 while adjusting the focus of illumination system 102 and observation system 104 to obtain multiple images with observation system 104 of virtual vertical slit 650 focused on construction circle 684 at spaced apart locations. The focus of each of illumination system 102 and observation system 104 may be adjusted as explained above in connection with FIG. 22 with R-Δ replacing R. As such, controller 300 may conduct an examination of eye 10 at multiple depths and multiple transverse locations without moving either illumination system 102 or observation system 104 relative to eye 10.

Once again, although in FIG. 22A, construction circle 684 is represented by a sphere of radius R-Δ, controller 300 may use other shapes to correspond to the shape of construction circle 684, such as aspheric curves, polynomial curves, and other shapes. The example provided in FIG. 22A is a non-limiting example.

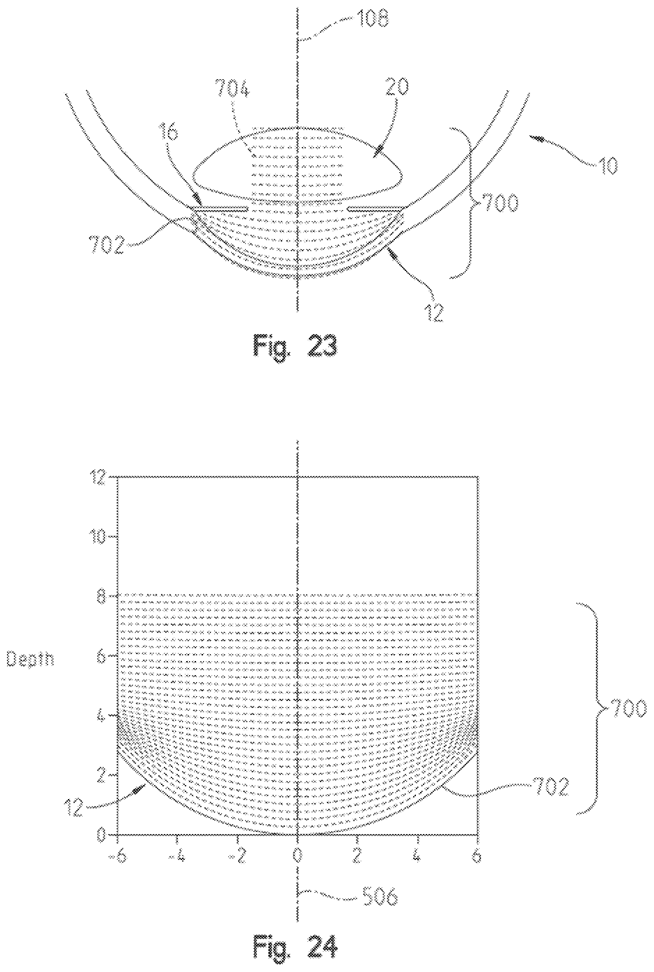

Referring to FIG. 23, a representation of a plurality of depth scan contours 700 are illustrated. As represented, a first contour 702 of the plurality of depth scan contours 700 generally matches cornea 12 and may be spherical similar to the examples in FIGS. 21 and 22 or aspherical or another suitable form. As the contours 700 are positioned deeper in eye 10, the contours 700 flatten out and either approach a linear form or are a linear form, see for example contour 704. An advantage of this, among others, is that deeper in the eye 10 structures that are to be imaged, such as the anterior surface of the crystalline lens as well as the iris are substantially flatter in comparison to the cornea. Additionally, in the case of pseudophakic eyes, the intraocular lens (IOL) as well as the remaining portion of the capsule are substantially flat. This approach allows the scan for the anterior and posterior surface of the cornea that are nearly concentric and the deeper structures (crystalline lens, iris, IOL) that are essentially flat. FIG. 24 shows another example of the plurality of contours 700. In embodiments, the contours 700 may flatten out as they progress from cornea 12 towards lens 20, approximate the anterior surface of lens 20 at a depth corresponding to the location of the anterior surface of lens 20, flatten out at depths within lens 20, and approximate the posterior surface of lens 20 at a depth corresponding to the location of the posterior surface of lens 20 (note the center of curvature of the contour corresponding to the posterior surface of lens 20 is closer to cornea 12 than the center of curvature of the contour corresponding to the anterior surface of lens 20). In embodiments, the reversal of curvature for the posterior surface of lens 20 is generated by controller 300 using a Gaussian function with a negative magnitude.

In embodiments, an average corneal and lens curvature data from a set of normal eyes or from a theoretical optical model of the eye such as the Gullstrand model of the eye is used by controller 300 to determine the plurality of depth scan contours 700.

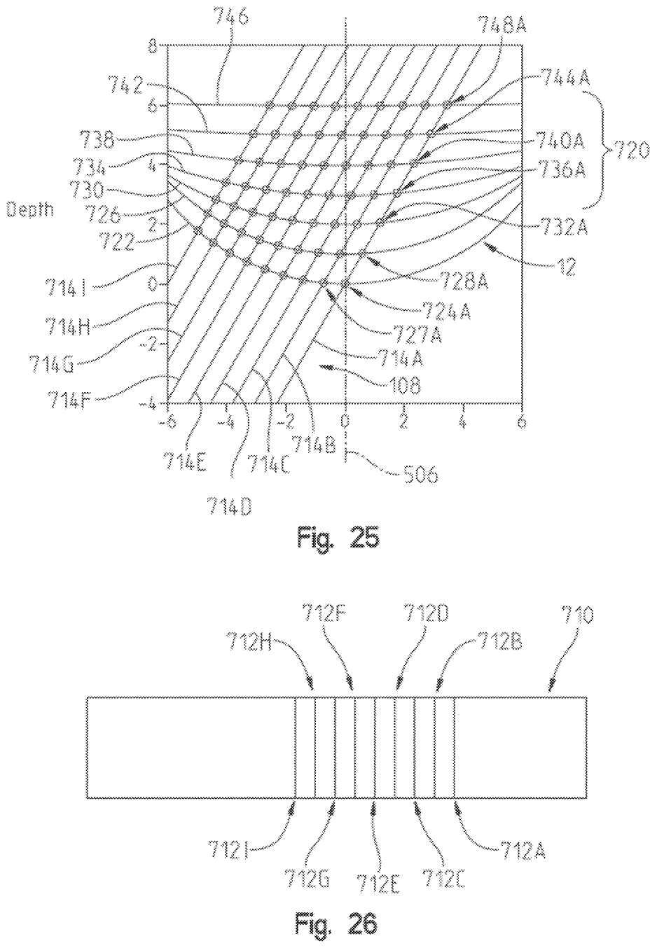

Referring to FIGS. 25 and 26, a plurality of example focal locations are shown for a plurality of contours 720. In the illustrated embodiment seven contours 722, 726, 730, 734, 738, 742, and 746 are shown which correspond to various depth scan profiles. In other embodiments, more or fewer contours 720 may be implemented. Referring to FIG. 26, an exemplary image 710 projected by illumination system 102, and in particular source 200, is shown. Although shown as a single image, in embodiments, image 710 is a composite of a plurality of different images each containing one of the plurality of virtual vertical slit 712A-I shown. In the illustrated embodiment, a first image would include virtual vertical slit 712A, a second image would include virtual vertical slit 712B, and so on for a total of nine separate images each projected independently and in embodiments sequentially. Although nine virtual vertical slits are shown, in embodiments, fewer or additional virtual vertical slits in fewer or additional images each projected independently and in embodiments sequentially. In embodiments, multiple spaced apart slits may be projected in the same image and exam logic 320 using image processing techniques may isolate the each respective slit in the images captured by the camera of observation system. An advantage, among others, of projecting multiple slits in the same image is to speed up data acquisition by exam logic 320.

Returning to FIG. 25, when the image containing virtual vertical slit 712A is projected by controller 300 onto eye 10 each of illumination system 102 and observation system 104 are focused at a position 724A for contour 722, at a position 728A for contour 726, a position 732A for contour 730, a position 736A for contour 734, a position 740A for contour 738, a position 744A for contour 742, and a position 748A for contour 746. Virtual vertical slit 712A is represented in FIG. 25 by ray line 714A. FIG. 25 further includes focal positions (not numbered, but represented by circles) for each of contours 720 for each of virtual vertical slits 712B-I which are represented by ray lines 714B-I. Thus, in the illustrated embodiment, exam logic 320 of controller 300 focuses each of illumination system 102 and observation system 104 at sixty-three separate focal locations without requiring movement of either of illumination system 102 or observation system 104 relative to eye 10. In other embodiments, exam logic 320 of controller 300 includes fewer or additional focal locations for an examination. In embodiments, at each of the points (such as 724A) shown in FIG. 25, a respective plane is imaged by observation system 500 that passes through the corresponding point and is normal to the optical axis 506 of the observation system 500. In embodiments, exam logic 320 aligns each of illumination system 102 and observation system 104 to the apex 514 of the cornea 12 of eye 10 in a first spatial configuration. While maintaining the illumination system 102 and the observation system 104 in the first spatial configuration, the exam logic 320 causes controller 300 to carry out the examination at the locations identified in FIG. 25. For example, controller 300 may perform the following steps: (a) projecting with the first adjustable optical assembly of the illumination system a first image from a digital projector towards the eye at a first focal depth (such as point 728A) relative to the apex of the cornea of the eye, the first image including a vertical slit in a first transverse position (such as slit 712A), (b) receiving light reflected by the eye from the projecting step (a) with a first camera through the second adjustable optical assembly of the observation system with the second adjustable optical assembly focused at the first focal depth relative to the apex of the cornea of the eye, (c) projecting with the first adjustable optical assembly the first image from the digital projector towards the eye at a second focal depth (such as point 732A) relative to the apex of the cornea of the eye, the second focal depth being different than the first focal depth, the first image including the vertical slit in the first transverse position, and (d) receiving light reflected by the eye from the projecting step (c) with the first camera through the second adjustable optical assembly with the second adjustable optical assembly focused at the second focal depth relative to the apex of the cornea of the eye. Controller continues on for various images of the vertical slit at various transverse positions and focal depths. In another example, controller 300 may perform the following steps (a) projecting with the first adjustable optical assembly of the illumination system a first image from a digital projector towards the eye at a first focal depth (such as point 728A) relative to the apex of the cornea of the eye, the first image including a vertical slit in a first transverse position (such as slit 712A), (b) receiving light reflected by the eye from the projecting step (a) with a first camera through the second adjustable optical assembly of the observation system with the second adjustable optical assembly focused at the first focal depth relative to the apex of the cornea of the eye, (c) projecting with the first adjustable optical assembly a second image from the digital projector towards the eye at a second focal depth (such as point 727A) relative to the apex of the cornea of the eye, the second focal depth being different than the first focal depth, the second image including the vertical slit in the second transverse position (such as slit 712B), the second transverse position being offset from the first transverse position, and (d) receiving light reflected by the eye from the projecting step (c) with the first camera through the second adjustable optical assembly with the second adjustable optical assembly focused at the second focal depth relative to the apex of the cornea of the eye.

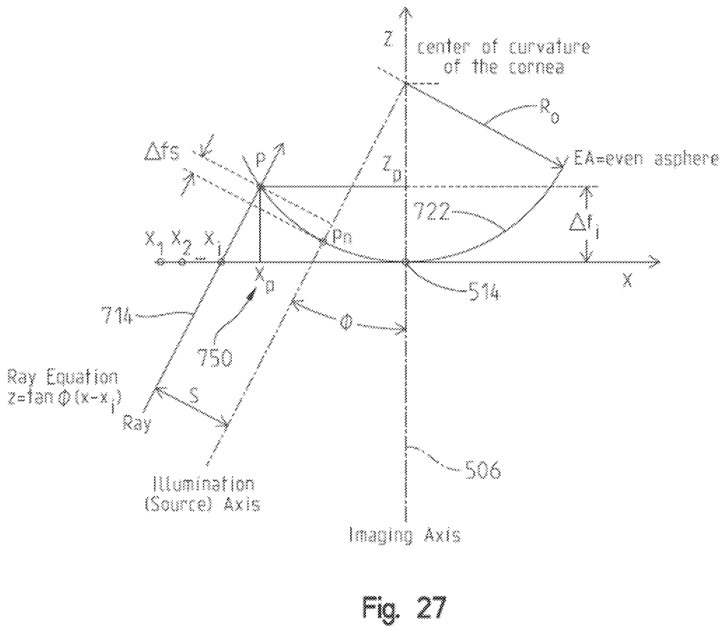

Referring to FIGS. 27-30, an exemplary process for defining the contour curves 720 and corresponding focal depths for illumination system 102 and observation system 104 are shown. Referring to FIG. 27, a generic contour, illustratively contour 722, is shown for the desired depth scan profile and a generic virtual vertical slit, illustratively represented as being projected along ray line 714 are shown.

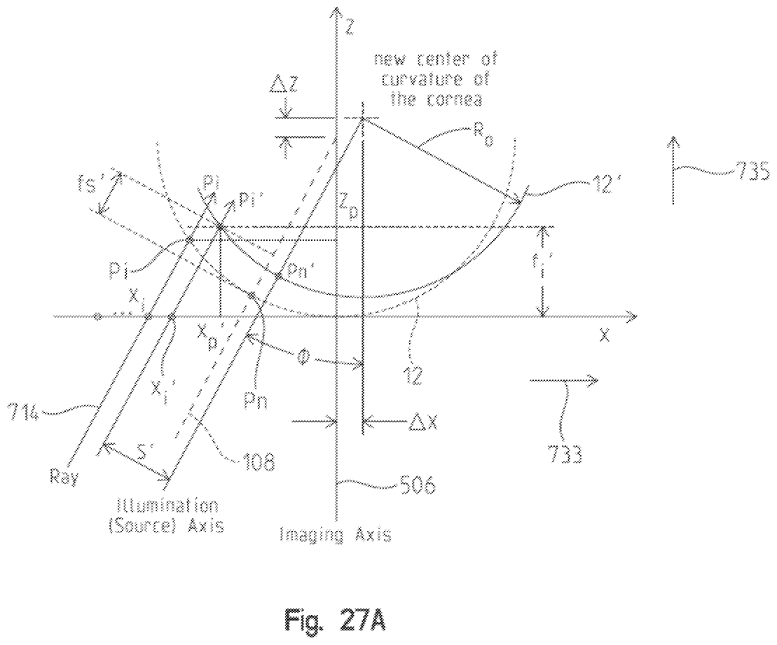

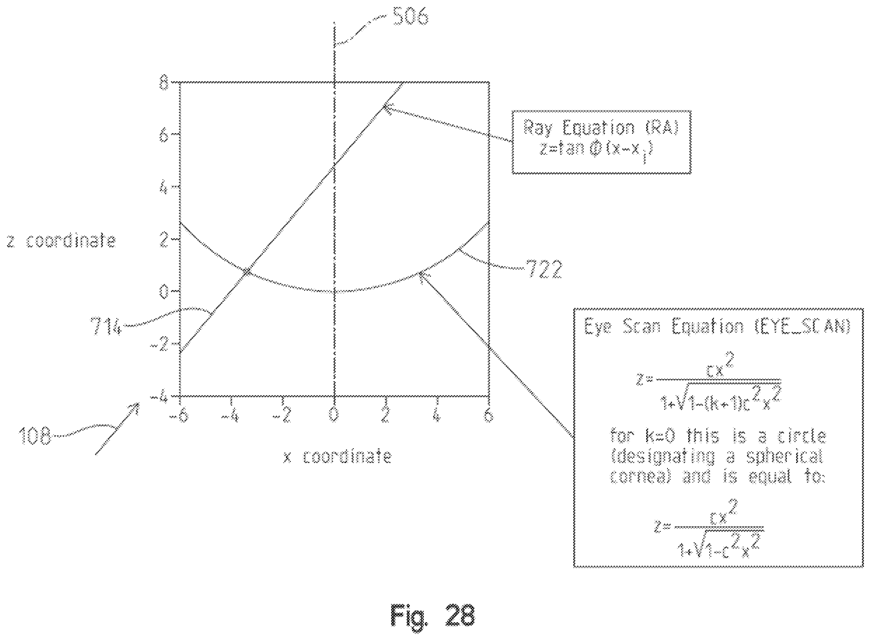

In FIG. 27 contour 722 is an aspheric. Referring to FIG. 28, contour 722 follows the form of the Eye Scan Equation (a variant of the even asphere surface in parametric form) shown in FIG. 27 which specifies the depth z for contour 722 at a given x value for a given curvature c (c=1/R) and a conical constant k (for k=0 the eye equation becomes a circle). The ray line 714 on which the virtual vertical slit is to be projected follows the form of the Ray Equation shown in FIG. 27 which species the depth z for various x values.

Now to determine the focal location for illumination system 102 and observation system 104, controller 300 determines the intersection of the Eye Scan Equation and the Ray Equation which is the focal depth for illumination system 102 and observation system 104. In embodiments, controller 300 determines the intersection by solving the set of equations, the Eye Scan Equation and the Ray Equation. In another embodiment, controller 300 determines the depth z for each of the Eye Scan Equation and the Ray Equation for a plurality of x values, as shown in FIG. 28. Next, controller 300 determines the absolute value of the difference between the Ray Equation and the Eye Scan Equation, as shown in FIG. 30. The global minimum of this difference is the x position 750 which corresponds to the Eye Scan Equation and the Ray Equation having the same depth z value. Returning to FIG. 27, this x position 750 is denoted as Xₚ and the corresponding depth position is denoted as Zₚ resulting in illumination system 102 being focused at point p having coordinates (Xₚ, Zₚ) and observation system 104 being focused at a depth of Zₚ. In embodiments, the initial focus of illumination system 102 is at point pₙ in FIG. 27, thus controller 300 determines the Δfₛ (Δsource) along the direction of ray line 714 from pₙ. Further, controller 300 determines Δfᵢ (Δobservation) from location 514 where observation system 104 was initially focused. Controller 300 repeats this process for each planned scan position along a given contour and at the various depths. In embodiments, controller 300 scans all planned scan positions on a given contour prior to moving to the next contour. In embodiments, the scan sequence may be optimized with respect to the required steps of the focusing mechanisms and therefore decrease the total time required for the scan. In embodiments, the sequence is optimized so that the focusing systems (e.g. tunable lenses) are changing essentially continuously in a quasi-static manner. In embodiments, controller 300 scans for a given ray line 714 all depths (each planned contour) prior to moving the slit to the next planned ray line.

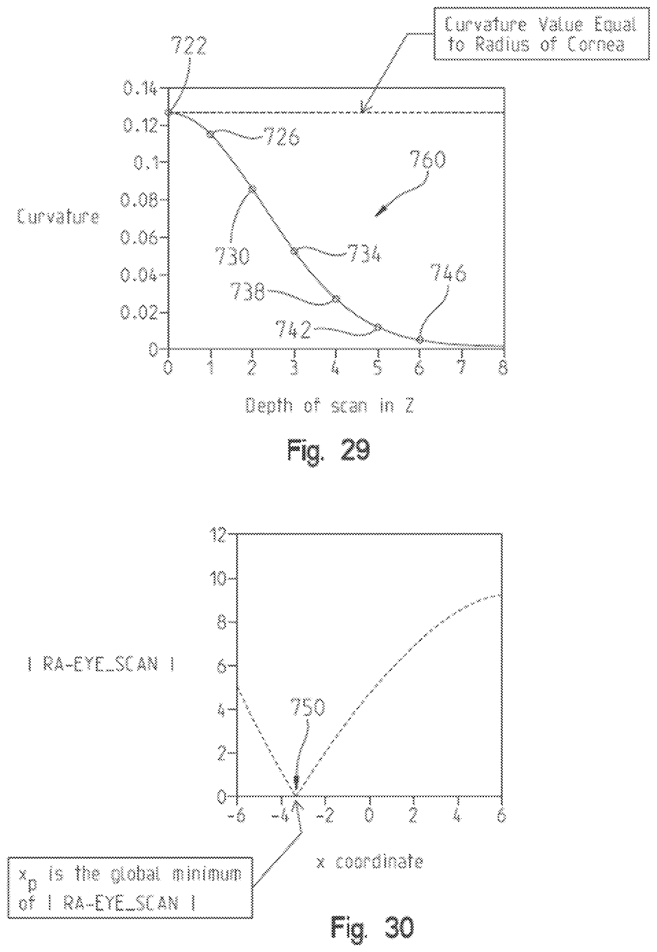

As shown in FIG. 25, in embodiments, as a planned scan goes deeper into the eye the scan contours become more linear and have less variation in depth. In embodiments, this flattening of the contour lines is controlled by controller 300 by altering the curvature value in the Eye Scan Equation (see FIG. 28) for each successive contour. In one example, controller 300 may linearly decrease the curvature value to flatten out the contours. In another example, controller 300 follows a Gaussian distribution, such as distribution 760 shown in FIG. 29 to control the flattening of the contours in the depth (z) direction. The curvature values corresponding to each of contours 722, 726, 730, 734, 738, 742, and 746 are shown in FIG. 29.

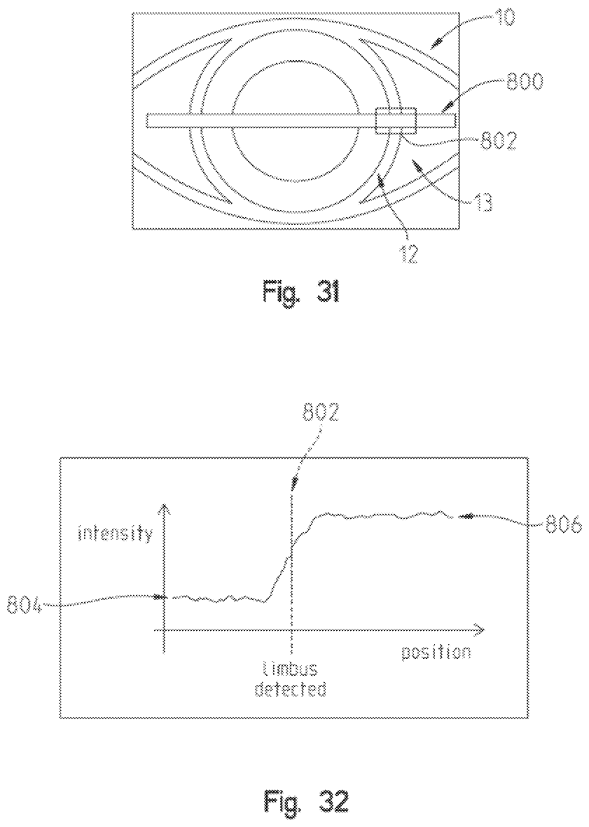

Examination system 100 may be used to determine additional characteristics for an eye 10 under examination. Referring to FIG. 31, eye 10 is illuminated with a virtual horizontal slit 800 which stretches across the field of view of observation system 104. Referring to FIG. 32, controller 300 may scan the intensity values received by first camera 502 to determine a location of a limbus 802 of eye 10. Limbus 802 is the intersection of the transparent cornea 12 of the eye 10 and the opaque sclera 13 of the eye 10. As shown in FIG. 32, the reflected light from the cornea 12 is generally at a first low level 804 and the reflected light from the sclera 13 is generally at a second high level 806. In embodiments, controller 300 determines when the reflected light has an intensity value halfway between level 804 and level 806 and that location corresponds to the location of the limbus 802. For virtual horizontal slit 800, controller 300 should detect both a left side limbus location and a right side limbus location. In embodiment, controller 300 may use the determined limbus locations to assist in aligning examination system 100 to eye 10.

Further, the determination of the location of the limbus 802 may be used to monitor if the subject moves their eye during an examination. Referring to FIG. 33A, controller 300 may project with source 200 a plurality of slits, illustratively a left upper slit 830 angled towards a center 828 of eye 10, a right upper slit 832 angled toward center 828 of eye 10, a left lower slit 834 angled toward center 828 of eye 10, and a right lower slit 836 angled toward center 828 of eye 10. The location of limbus 802 may be determined by controller 300 for each of these slits, left upper slit 830, right upper slit 832, left lower slit 834, and right lower slit 836. If the location of limbus 802 does not change for any of left upper slit 830, right upper slit 832, left lower slit 834, and right lower slit 836, then controller 300 determines eye 10 has not moved.

If the location of limbus 802 moves outward away from center 828 for left upper slit 830 and right upper slit 832 and inward towards center 828 for left lower slit 834 and right lower slit 836, controller 300 determines that eye 10 has moved upward in direction 840. If the location of limbus 802 moves outward away from center 828 for left lower slit 834 and right lower slit 836 and inward towards center 828 for left upper slit 830 and right upper slit 832, controller 300 determines that eye 10 has moved downward in direction 842. The movement of eye 10 in direction 840 or direction 842 may be determined with only two of the four of left upper slit 830, right upper slit 832, left lower slit 834, and right lower slit 836.

If the location of limbus 802 moves outward away from center 828 for right upper slit 832 and right lower slit 836 and inward towards center 828 for left upper slit 830 and left lower slit 834, controller 300 determines that eye 10 has moved leftward in direction 844. If the location of limbus 802 moves inward towards center 828 for right upper slit 832 and right lower slit 836 and outward away from center 828 for left upper slit 830 and left lower slit 834, controller 300 determines that eye 10 has moved rightward in direction 846. The movement of eye 10 in direction 844 or direction 846 may be determined with only two of the four of left upper slit 830, right upper slit 832, left lower slit 834, and right lower slit 836.

In embodiments, controller 300 utilizes at least a portion of left upper slit 830, right upper slit 832, left lower slit 834, and right lower slit 836 to monitor for eye movement while scanning a virtual vertical slit, such as virtual vertical slit 650, across eye 10 as described herein. In embodiments, if an eye movement is detected, controller 300 determines the direction (as mentioned above) and magnitude of the movement with at least a portion of left upper slit 830, right upper slit 832, left lower slit 834, and right lower slit 836 and adjusts the focal positions of the illumination system and observation system for virtual vertical slit 650 based thereon to continue the exam. In embodiments, if an eye movement is detected, controller 300 monitors for a first period of time to see if eye 10 returns to its original position and if so continues with the examination with virtual vertical slit 650 and if not stops the examination, and optionally provides an alert to the operator.

In embodiments, other features of the eye 10 under examination may be used by controller 300 to track eye movement. For example, controller 300 may determine the relatively round margin of the pupil of the eye 10 or the limbus and track their location from frame to frame, such as determining a center of one or each and tracking this center location from frame to frame.

If it is determined that the eye 10 of the patient has moved, exam logic 320 updates the positions of the scan to account for the determined movement. Referring to FIG. 27A, source axis 108 and imaging axis 506 are shown, as in FIG. 27, with imaging axis 506 intersecting the apex of the cornea 12. The eye 10 has moved to a different location denoted by cornea 12′. The location of cornea 12′ is both translated to the right in direction 733 along the x-axis and rearward in direction 735 along the z-axis. This results in point Pₙ (intersection of source axis 108 with cornea 12) also being shifted to a new location denoted P_{n′} and a representative scan location Pᵢ also being shifted to a new location denoted by P_{i′.}

By utilizing one or more of the techniques mentioned herein, exam logic 320 is able to determine an offset Δx in direction 733 and an offset Δz in direction 735. With these offsets, exam logic 320 may determine updated values for f_{s′} relative to Pₙ and for f_{i′} relative to the x-axis. Exam logic 320 may update the values for each scan point based on the determined movement of the eye 10. In embodiments, examination system 100 includes first camera 502 or an additional camera used to track a feature of eye 10, such as the pupil or limbus or iris, and includes a stereo camera pair, such as cameras 504 and 504′ in FIG. 7A to track a depth from examination system 100 to eye 10. As such, first camera 502 or an additional camera used to track a feature of eye 10, such as the pupil or limbus or iris can provide an indication of an offset Δx in direction 733 and a stereo camera pair, such as cameras 504 and 504′ in FIG. 7A, can provide an indication of an offset Δz in direction 735.

In embodiments, exam logic 320 of controller 300 analyzing captured images to determine if the subject has blinked. For example, a blurred image may be associated with a blink and controller 300 maintains the projected slit 712 and cameras at the current focus depths until a focused image is obtained and thereafter subsequently moves on to the next location in the scan being performed by exam logic 320 of controller 300.

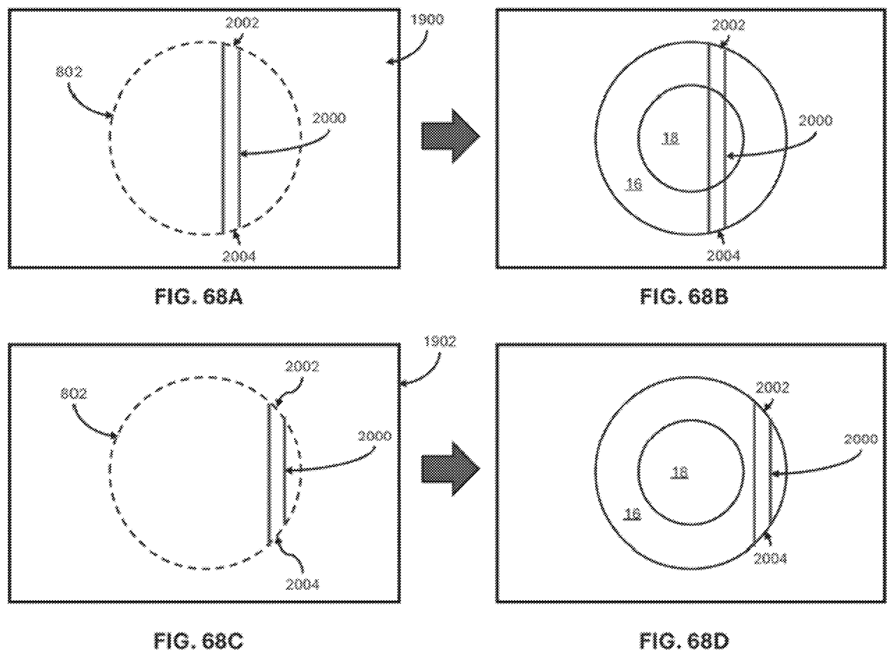

In embodiments, exam logic 320 of controller 300 identifies the limbus 802 and limits a vertical extent of slit 712 to not extend beyond the perimeter of the limbus 802. A diameter and center 828 of the limbus may be determined with a test pattern, such as shown in FIG. 33B. Controller 300 then knowing the position of the slit 712 to be projected may determine the intersection with the limbus 802 and truncate the slit 712 to not extend beyond the limbus 802. An advantage, among others, of truncating slit 712 is to avoid distractions to the viewer of observation system 104 from overexposed sclera regions in the captured images. Referring to FIGS. 68A-D, an example is shown. Referring to FIG. 68A, the determined location of limbus 802 is represented by a dashed line. A projected image 1900 including a test pattern, a vertical slit 2000, is shown. Vertical slit 2000 includes a truncated top portion 2002 and a truncated bottom portion 2004, each of which generally align with the intersection of vertical slit 2000 and the determined location of limbus 802. As shown in FIG. 68B, vertical slit 2000 is projected on eye 10 and generally transverses the iris 16 and pupil 18 or generally equally the vertical extent of cornea 12. Referring to FIG. 68C, as exam logic 320 of controller 300 moves vertical slit 2000 in the transverse direction, illustratively to the right relative to the position in FIG. 68A, exam logic 320 of controller 300 updates the intersection of the determined location of limbus 802 and vertical slit 2000 and projects image 1902. As shown in FIG. 68D, the vertical extent of vertical slit 2000 is shorter in FIG. 68D then in FIG. 68B.

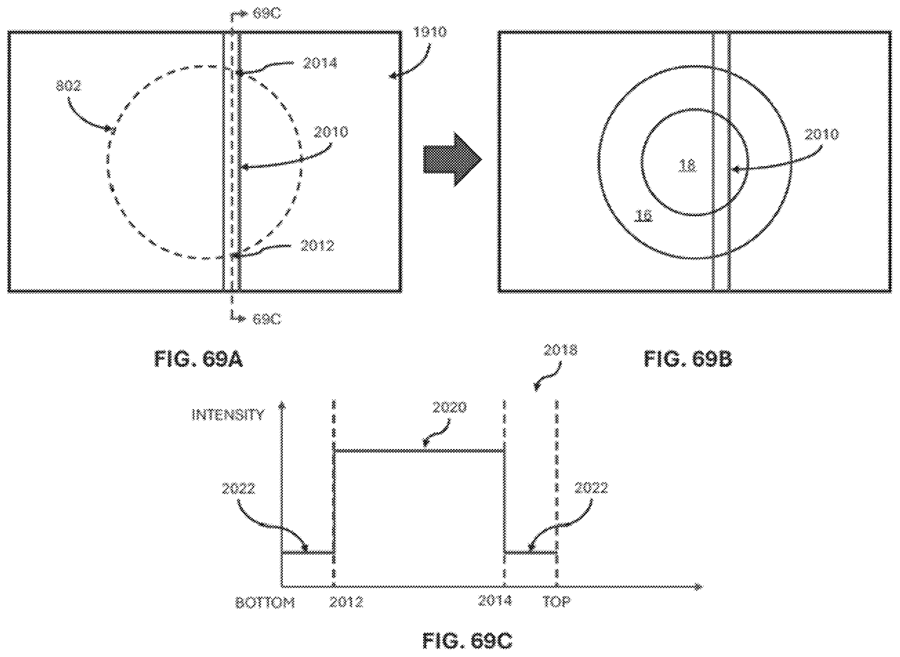

Further or alternatively, a brightness level of slit 712 may be altered along its length to dim regions outside of the limbus 802 and/or in the perilimbal area prior to reaching the actual limbus. As such, in one embodiment, slit 712 would have a first brightness level across the cornea, at least one lower brightness level at the edge of the limbus and outside of the limbus. In another embodiment, slit 712 would have a first brightness level across the cornea, at least one lower brightness level at the edge of the limbus, and be truncated outside of the limbus. Referring to FIGS. 69A and 69C, exam logic 320 of controller 300 projects an image 1910 with a vertical slit 2010. An exemplary intensity profile 2018 of vertical slit 2010 along its vertical extent is shown in FIG. 69C. Intensity profile 2018 has a first intensity level 2020 in the region that corresponds to the determined location of limbus 802 (between point 2012 and point 2014) and a lower intensity level 2022 in a region below point 2012 and in a region above point 2014. By having these multiple intensity levels in intensity profile 2018, the viewed image of light reflected from eye 10 will have a first brightness level across the cornea and a lower brightness level at the edge of the limbus and outside of the limbus. In embodiments, the lower region from the bottom to point 2012 and the upper region from point 2014 to the top have different intensity levels. In embodiments, intensity level 2020 is not a constant intensity level across the cornea 12 (within the determined location of the limbus), but rather includes multiple intensity levels. In embodiments, intensity level 2020 has a first value at point 2012 and rises to a midpoint and falls back to the first value at point 2014.

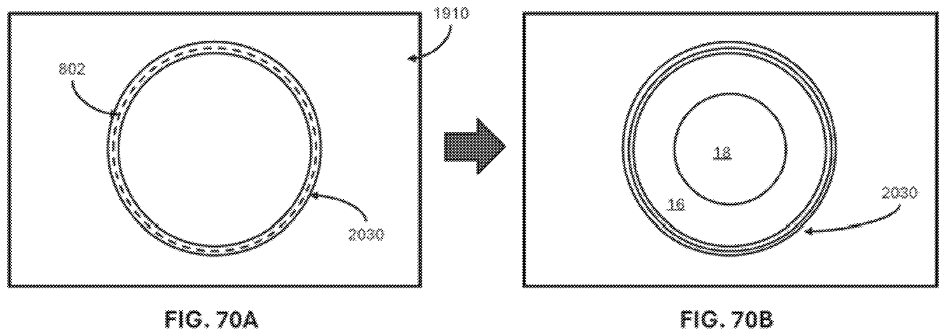

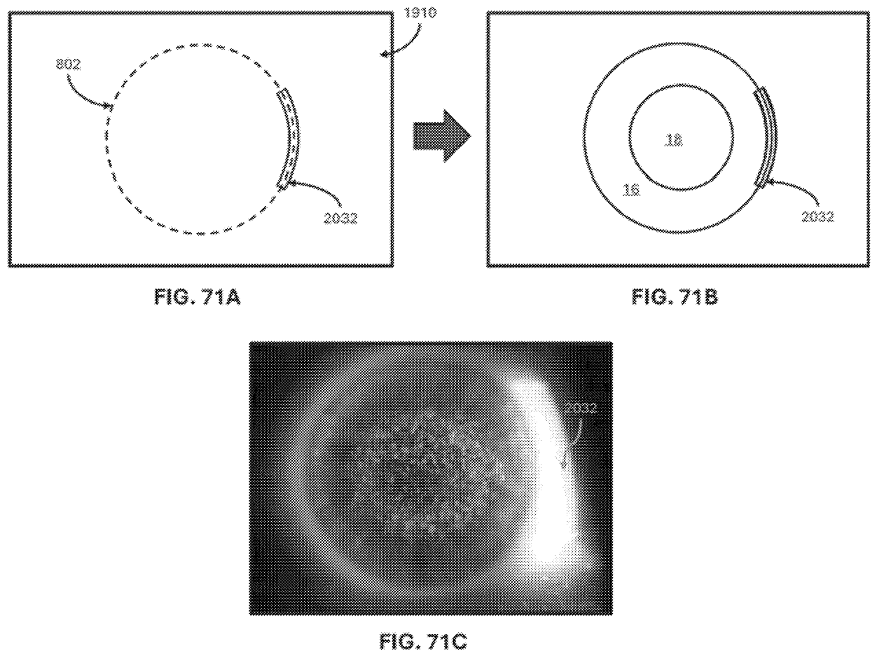

In embodiments, exam logic 320 of controller 300 identifies the limbus 802. A diameter and center 828 of the limbus may be determined with a test pattern, such as shown in FIG. 33B. Controller 300 then knowing that the limbus has a diameter of “D” may instruct illumination system 102 to generate a test pattern with projector 402 which includes a ring of light 2030 (see FIG. 70A) or a partial ring of light 2032 (see FIG. 71A) having a diameter equal to about “D” and to project the ring of light or partial ring of light onto eye 10 with the ring of light or partial ring of light generally aligned with the limbus (see FIGS. 70B and 71B). This will cause a portion of the light to internally reflected within cornea 12 and allow the visualization of subtle corneal scars or other pathology. In embodiments, a gradient transition zone, such as a ramp is employed across a width of the ring or partial ring to limit overexposure of the perilimbal cornea which often has some degree of arcus senilis which will reflect more light back to the camera due to its white color.

In embodiments, a thickness of the ring of light or partial ring of light is about 1 millimeter. Referring to FIG. 71C, an example of partial ring of light 2032 projected on a right side of eye 10 is shown. As shown in FIG. 71C, sclerotic scatter is generated. An advantage, among others, of using partial ring of light 2032 instead of a traditional vertical slit is that more of the projected light is impinging the limbus which increases the amount of scatter and reduces the amount of stray illumination in other parts of the image.

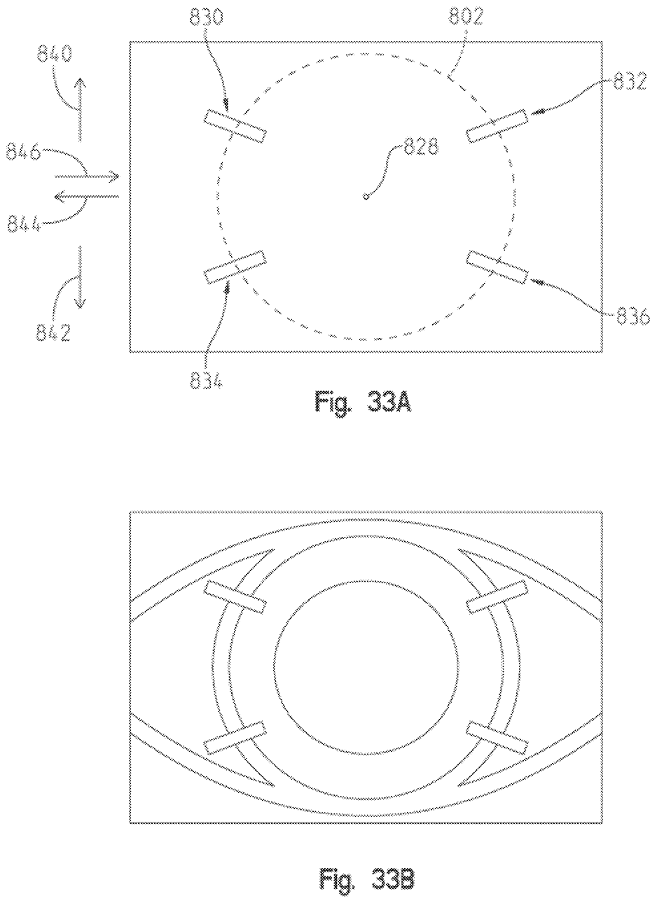

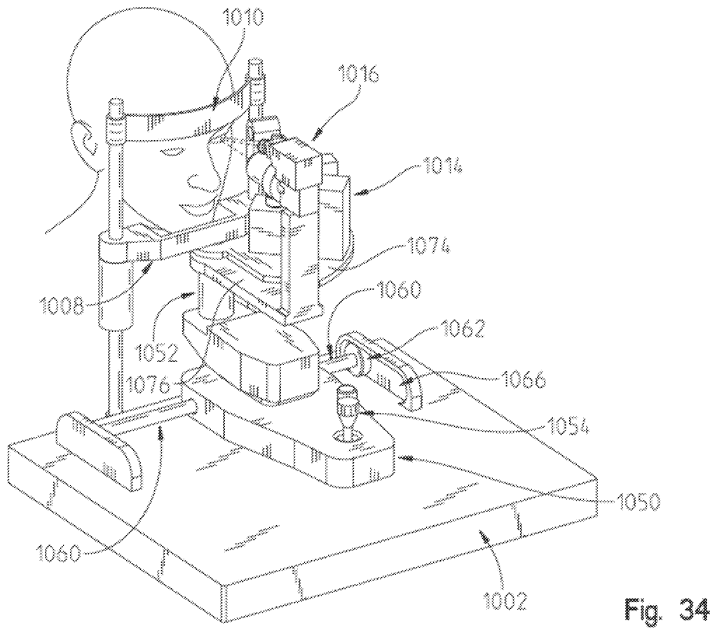

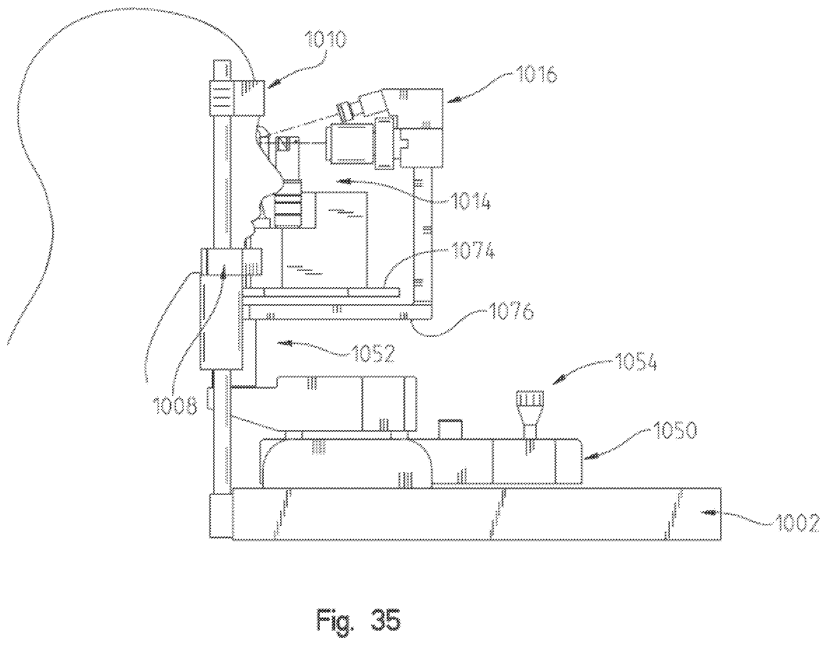

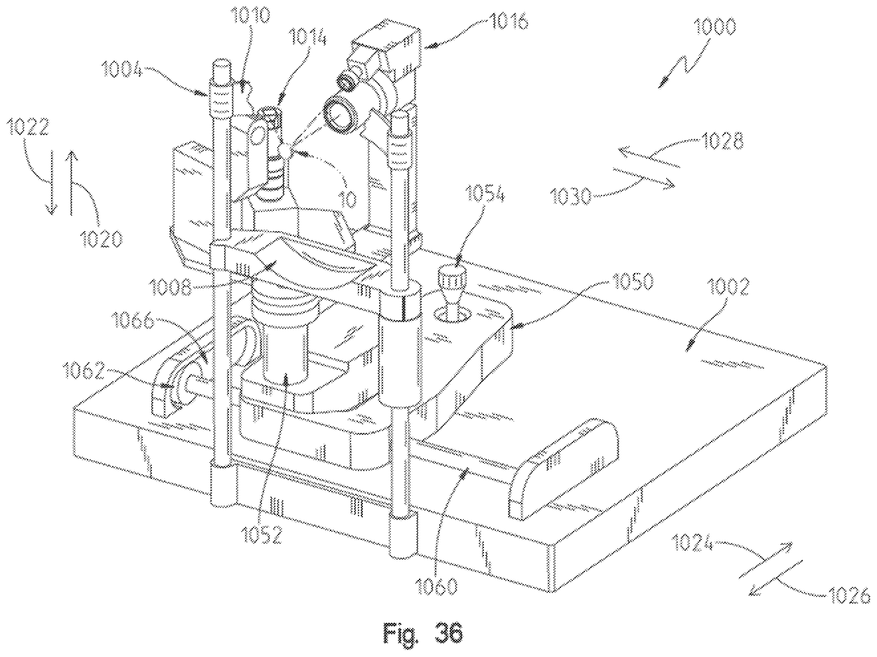

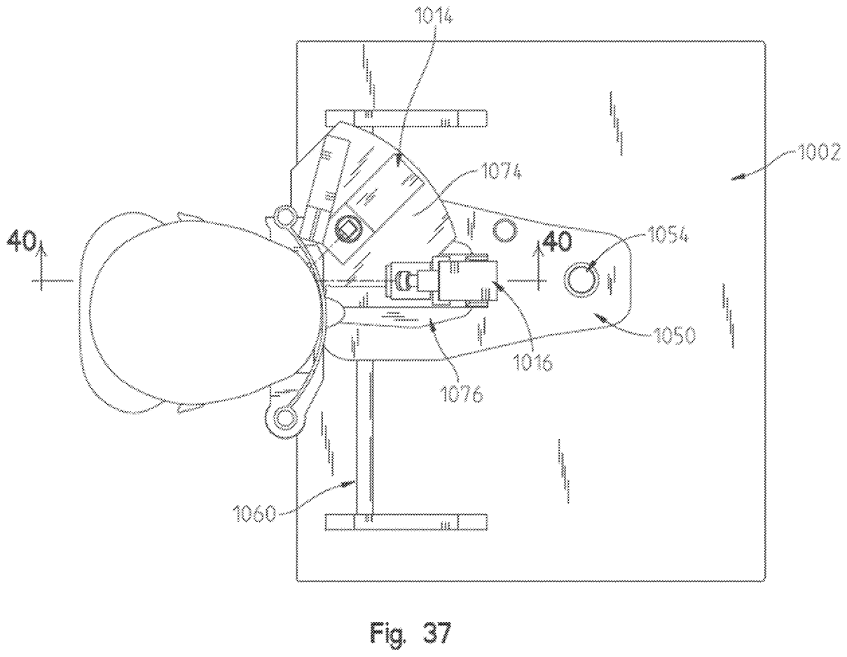

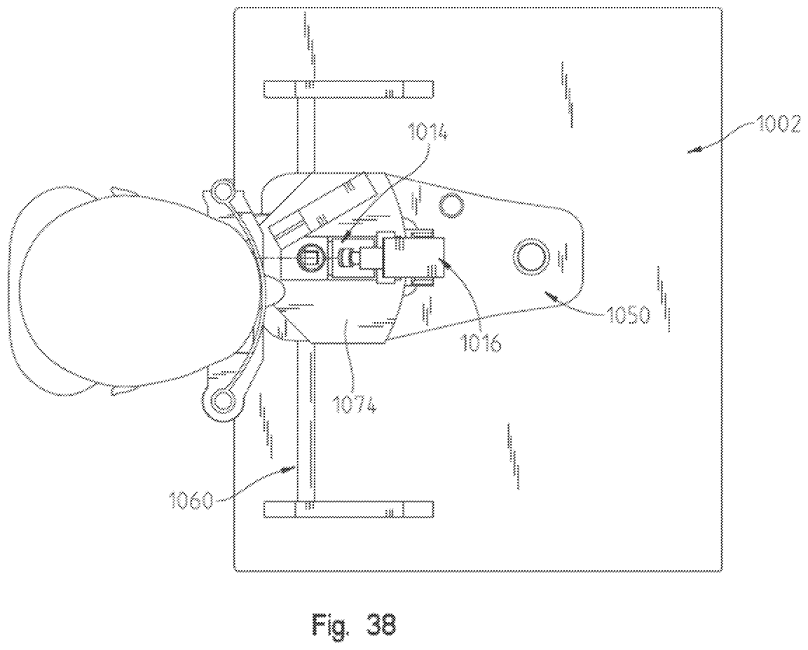

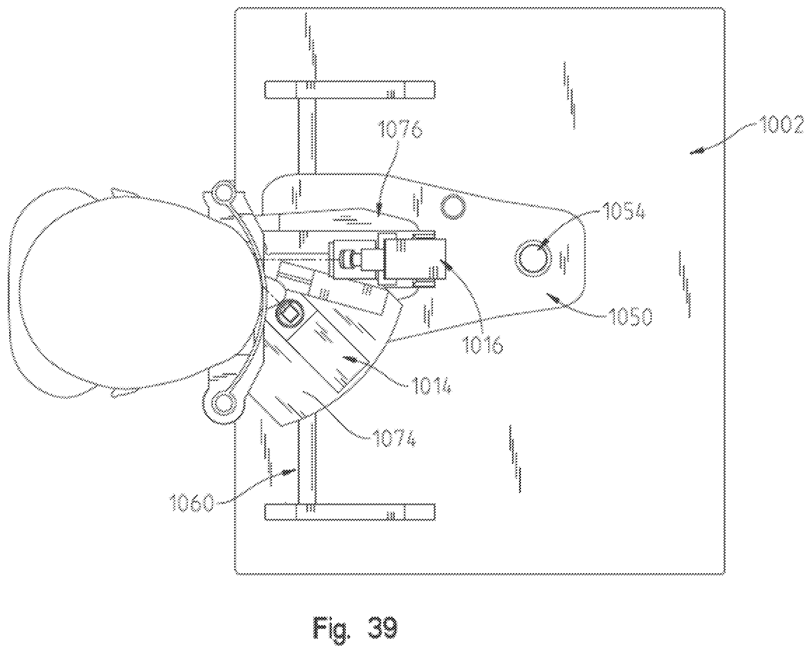

Referring to FIGS. 34-42, an exemplary examination system 1000 is shown. In embodiments, examination system 1000 operates in accordance with the descriptions of examination system 100 herein.

Referring to FIGS. 34-36, examination system 1000 includes a base 1002 to which a patient support 1004 is coupled. Patient support 1004 includes a pair of upstanding members 1006 which support a chin rest 1008 and a forehead rest 1010. Chin rest 1008 and forehead rest 1010 are moveable up in direction 1020 and down in direction 1022 (y-axis) to vertically position eye 10 of a patient relative to an illumination system 1014 and an observation system 1016 of examination system 1000.

Each of illumination system 1014 and observation system 1016 are supported by a movable base 1050 which in turn is supported by base 1002. Moveable base 1050 is moveable relative to base 1002 in direction 1024 away from eye 10 and direction 1026 towards eye 10 (z-axis) and in direction 1028 and direction 1030 (x-axis). The movement of moveable base 1050 relative to base 1002 results in the movement of both illumination system 1014 and observation system 1016. In the illustrated embodiments, movable base 1050 is moved in the x-axis and z-axis by the operator grasping input 1054 and moving movable base 1050. Movable base 1050 receives a shaft 1060 and is translatable along shaft 1060 in direction 1028 and direction 1030 to move movable base 1050 relative to base 1002 in the x-axis. Further, each end of shaft 1060 has a wheel 1062 coupled thereto which is received in a race 1066 of base 1002. Each wheel 1062 rolls in the respective race 1066 in direction 1024 and direction 1026 to move movable base 1050 relative to base 1002 in the z-axis. In one embodiment, one of illumination system 1014 and observation system 1016 is not supported by moveable base 1050 and thus does not move in concert with moveable base 1050 when moveable base 1050 is moved relative to base 1002.

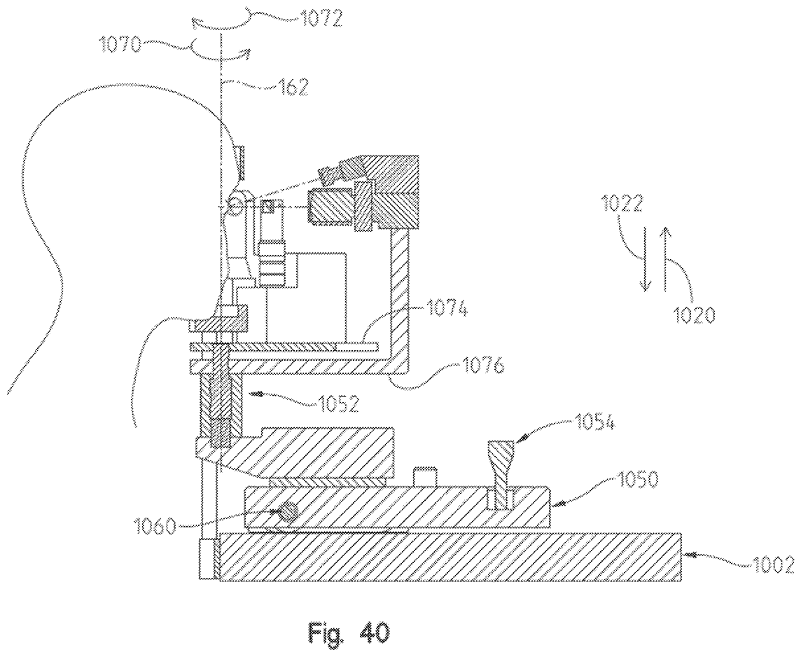

Illumination system 1014 and observation system 1016 are both supported on a column 1052 of movable base 1050. Further, referring to FIG. 40, each of illumination system 1014 and observation system 1016 are rotatable relative to moveable base 1050 in direction 1070 and direction 1072 about axis 162. Illumination system 1014 is supported on a platform 1074 and observation system 1016 is supported on a platform 1076. Each of platform 1074 and platform 1076 are individually rotatable relative to moveable base 1050. As such, illumination system 1014 may be rotated relative to moveable base 1050 without a corresponding rotation of observation system 1016 relative to moveable base 1050 or vice versa.

Although illumination system 1014 and observation system 1016 are shown being rotatable about a vertical axis, axis 162, one or both of illumination system 1014 and observation system 1016 may be rotatable about a horizontal axis parallel to the x-axis or another axis in a plane defined by the x-axis and the y-axis. In one embodiment, each of illumination system 1014 and observation system 1016 is rotatable about a separate axis relative to moveable base 1050. In the illustrated embodiment, illumination system 1014 and observation system 1016 are each individually rotatable relative to movable base 1050 about axis 162 and are rotatable relative to each other about axis 162.

Referring to FIG. 41, illumination system 1014 is positionable relative to eye 10 to project virtual vertical slit 650 on eye 10. In embodiments, illumination system 1014 includes the components of illumination system 400 and operates as discussed herein. In embodiments, observation system 1016 includes the components of observation system 500 and operates as discussed herein. As shown in FIG. 41, axis 506 of first camera 502 and axis 510 of second camera 504 intersect at location 514.

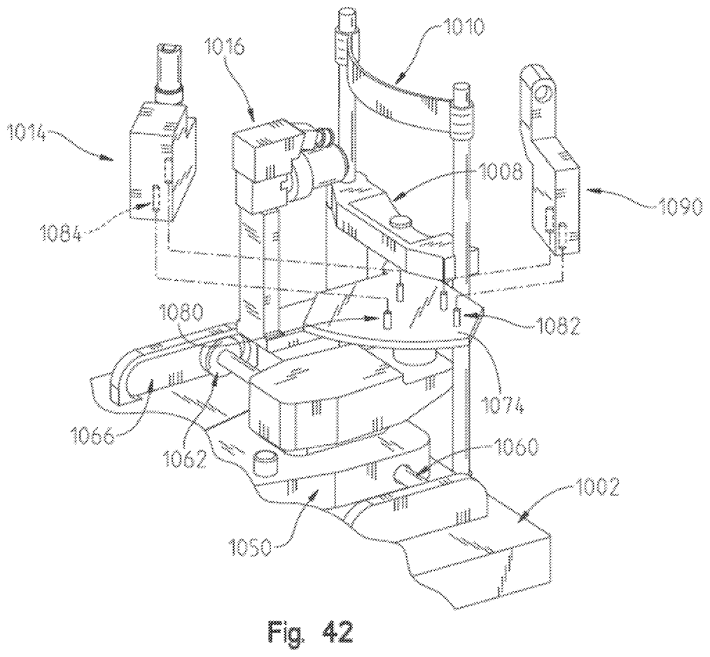

Referring to FIG. 42, in embodiments, platform 1074 includes mounts 1080 and 1082, illustratively pins, each of which may interface with corresponding mounts 1084 to secure illumination system 1014 to platform 1074. Thus, illumination system 1014 may be positionable at various locations on platform 1074. Further, additional components, such as a beamspliter 1090, or other components, such as such as OCT, fundus camera, intraocular lens biometer, topogropher, contact or non-contact tonometer, specular microscope (for imaging the endothelium) and other suitable devices. In embodiments, component 1090 may be a support for an additional lens or lenses, such as an aspheric lens to enhance a visualization of the central retina of eye 10 with first camera 502.

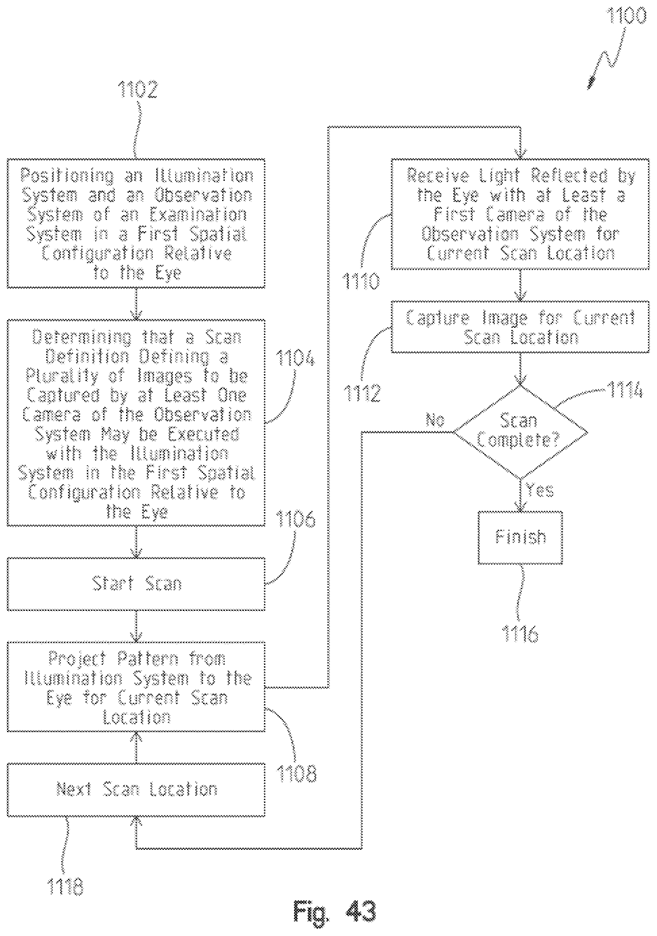

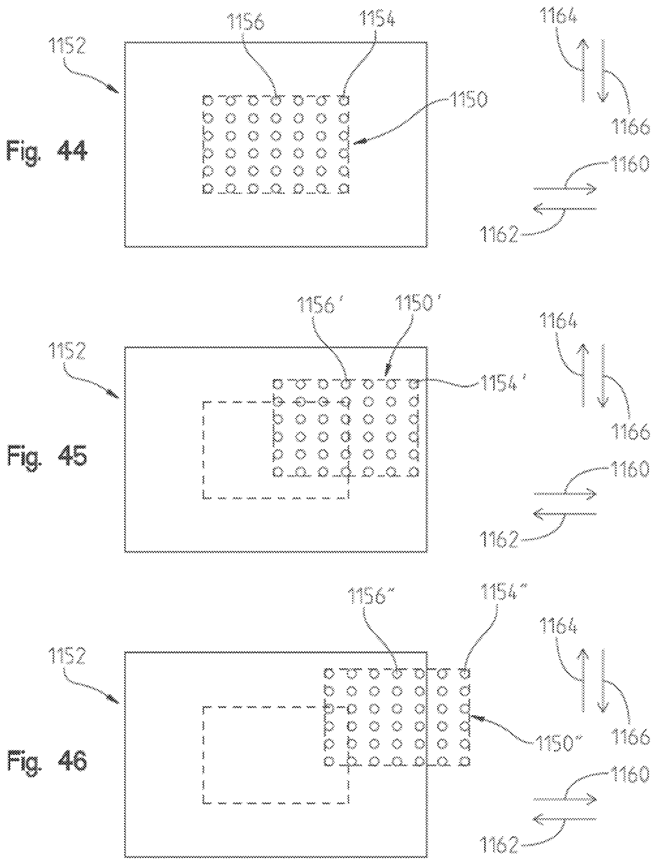

Referring to FIG. 43, an exemplary processing sequence 1100 for exam logic 320 is illustrated. Illumination system 102 and observation system 104 of examination system 100 are positioned by controller 300 executing exam logic 320 in a first spatial configuration relative to the eye, as represented by block 1102. In embodiments, the first spatial configuration corresponds with illumination system 102 and observation system 104 being positioned so that optical axis 108 of illumination system 102 and axis 506 of observation system 104 intersect as shown in FIG. 27. Controller 300 determines if a scan definition 1150 (see FIG. 44) defining a plurality of images to be captured by at least one camera of the observation system 104 may be executed with the illumination system 102 and the observation system 104 in the first spatial configuration relative to the eye 10, as represented by block 1104. In embodiments, scan definition 1150 is stored in memory 312 and selected by an operator for execution with examination system 100. In embodiments, scan definition 1150 is received from a remote controller. In examples, the plurality of images of scan definition 1150 include a first image at a first focal plane of the eye and a second image at a second focal plane of the eye, the second focal plane being offset from the first focal plane. In another example, the plurality of images of scan definition 1150 include a first image and a second image both at a first focal plane of the eye.

Referring to FIG. 44, a representation of a scan envelope 1152 of examination system 100 and a current scan definition 1150 is represented. Scan envelope 1152 represents the limits of examination system 100 to project desired test patterns on eye 10 with illumination system 102 and image those projected test patterns with observation system 104 when illumination system 102 and observation system 104 are in a first spatial configuration. An exemplary first spatial configuration is observation system 104 directly in front of eye 10 and illumination system 102 rotated sixty degrees to the left of observation system 104. Scan envelope 1152 is set based on the field of view of illumination system 102 and observation system 104 (which generally correspond to the horizontal limits in directions 1160 and 1162) and the focal depth adjustments capable for each of illumination system 102 and observation system 104 (which generally correspond to the vertical limits in directions 1164 and 1166). In embodiments, focal depth adjustments may be made by altering one or more tunable lens of illumination system 102 and/or observation system 104, by adjusting the relative positions of one or more lenses of illumination system 102 and/or observation system 104, or by a combination of altering one or more tunable lens of illumination system 102 and/or observation system 104 and adjusting the relative positions of one or more lenses of illumination system 102 and/or observation system 104. Although shown as a quadrilateral, scan envelope 1152 may take on different shapes.

Scan definition 1150 is shown centered in scan envelope 1152. This arrangement corresponds to examination system 100 being aligned with eye 10 as discussed herein. Scan definition 1150 includes a plurality of planned positions (also referred to herein as test locations and scan locations), one marked as position 1154 and one marked as position 1156. In embodiments, the positions correspond to the positions that illumination system 102 and observation system 104 are to be focused at relative to the eye 10, such as shown in FIGS. 25 and 27. Although the positions are shown as seven columns of six positions each that are equally spaced, any number and arrangement of positions may form scan definition 1150. In embodiments, scan definition 1150 includes positions which follow a contour of various eye structures as described herein or other arrangements. The boundary of scan definition 1150 is set based on the locations of the various positions within 1150 and need not be quadrilateral in shape.

Referring to FIG. 45, either an initial misalignment of eye 10 relative to examination system 100 is detected or a movement of eye 10 relative to examination system 100 is detected. In the illustrated example, scan definition 1150 is shifted in direction 1160 indicative of a misalignment or a movement of the eye 10 to the right relative to examination system 100 and is shifted upward in direction 1164 which is indicative of a misalignment or movement of the eye 10 away from examination system 100 along axis 506.

Exam logic 320 detects the offset of the eye 10 relative to examination system 100 (see FIG. 27A) and then determines revised positions for each scan position in the scan definition. The revised positions (such as position 1154′ and position 1156′ in FIG. 45) and overall revised scan definition 1150′. As illustrated in FIG. 45, each of the revised positions of scan definition 1150′ are still within scan envelope 1152 and thus examination system 100 is able to execute the desired scan while illumination system 102 and observation system 104 remain in the first spatial configuration relative to eye 10. Therefore, exam logic 320 causes the desired scan to commence. In embodiments, exam logic 320 provides an indicator to an operator that the desired scan can be performed and waits for an operator input to proceed. Exemplary inputs include a button press, a selection of a region on a display, a switch, or other suitable inputs. Exemplary indicators that the desired scan may be performed include visual indicators, audible indicator, tactile indicators, or combinations thereof. In one example, a green circle is shown surrounding at least a portion of the eye on a display as a visual indicator.

In embodiments, exam logic 320 continues to update the scan definition 1150 for each movement of eye 10 during the examination scan. If a movement or an initial misalignment causes scan definition 1150 to at least partly fall outside of scan envelope 1152, exam logic 320 either stops the examination scan or pauses the examination scan to move illumination system 102 and observation system 104 relative to eye 10 to either realign illumination system 102 and observation system 104 with eye 10 or to position illumination system 102 and observation system 104 relative to eye 10 such that scan definition 1150 is once again within scan envelope 1152. If moved, the moved illumination system 102 and observation system 104 are now in a second configuration. In embodiments, examination system 100 includes moveable base 140 which is movable by controller 300 to position illumination system 102 and observation system 104 relative to eye 10. In embodiments, moveable base 140 is manually moved by an operator to reposition illumination system 102 and observation system 104 relative to eye 10.

Referring to FIG. 46, either an initial misalignment of eye 10 relative to examination system 100 is detected or a movement of eye 10 relative to examination system 100 is detected. In the illustrated example, scan definition 1150 is shifted in direction 1160 indicative of a misalignment or a movement of the eye 10 to the right relative to examination system 100 and is shifted upward in direction 1164 which is indicative of a misalignment or movement of the eye 10 away from examination system 100 along axis 506.

As previously mentioned, exam logic 320 detects the offset of the eye 10 relative to examination system 100 (see FIG. 27A) and then determines revised positions for each scan position in the scan definition. The revised positions (such as position 1154″ and position 1156″ in FIG. 46) and overall revised scan definition 1150″. As illustrated in FIG. 46, a first portion of the scan positions including position 1154″are positioned outside of scan envelope 1152 and a second portion of the scan positions including position 1156″ are positioned within scan envelope 1152. In response exam logic 320 either stops the examination scan or pauses the examination scan to move illumination system 102 and observation system 104 relative to eye 10 to either realign illumination system 102 and observation system 104 with eye 10 or to position illumination system 102 and observation system 104 relative to eye 10 such that scan definition 1150 is once again within position 1154. In embodiments, exam logic 320 only updates scan definition 1150″ for the positions not yet scanned. As such, if all of the first portion shown in FIG. 46 had already been scanned, exam logic 320 would allow the examination scan to continue for the second portion of scan definition 1150″.

Returning to FIG. 43, once exam logic 320 determines that scan definition 1150 may be executed with illumination system 102 and observation system 104 in the first spatial configuration relative to eye 10, exam logic 320 begins the examination scan as represented by block 1106. In embodiments, exam logic 320 provides an indicator to an operator that the desired scan can be performed and waits for an operator input with input devices 360 to proceed. Exemplary inputs include a button press, a selection of a region on a display, a switch, or other suitable inputs. Exemplary indicators that the desired scan may be performed include visual indicators, audible indicator, tactile indicators, or combinations thereof. In one example, a green circle is shown surrounding at least a portion of the eye 10 on a display.

Exam logic 320 causes illumination system 102 to project a pattern towards the eye for the current scan location, as represented by block 1108. As discussed herein, in embodiments, illumination system 102 includes a digital projector that projects an image including a pattern, such as a vertical slit. The location of the vertical slit relative to an edge of the projected image may be different for different scan locations. In embodiments, exam logic 320 focuses the pattern at a focal plane intersecting the current scan position by adjusting one or more characteristics of illumination system 102. Exemplary characteristics include a tunable lens of illumination system 102.

Exam logic 320 focuses observation system 104 at a focal plane intersecting the current scan location so that observation system 104 may receive light reflected by eye 10 with at least one cameral of observation system 104 for the current scan location, as represented by block 1110. At least one image of the current scan location is captured by observation system 104, as represented by block 1112. The captured image or images may then be stored on memory 312 of controller 300. The captured image or images may have associated information stored therewith. Exemplary associated information includes the scan location coordinates, the parameters of examination system 100, patient identifying information, date and time information, notes, and additional information.

Exam logic 320 determines if additional scan positions in the scan definition need to be scanned, as represented by block 1114. If not, processing sequence 1100 ends, as represented by block 1116. If so, exam logic 320 determines the next scan location, as represented by block 1118, and returns to block 1108.

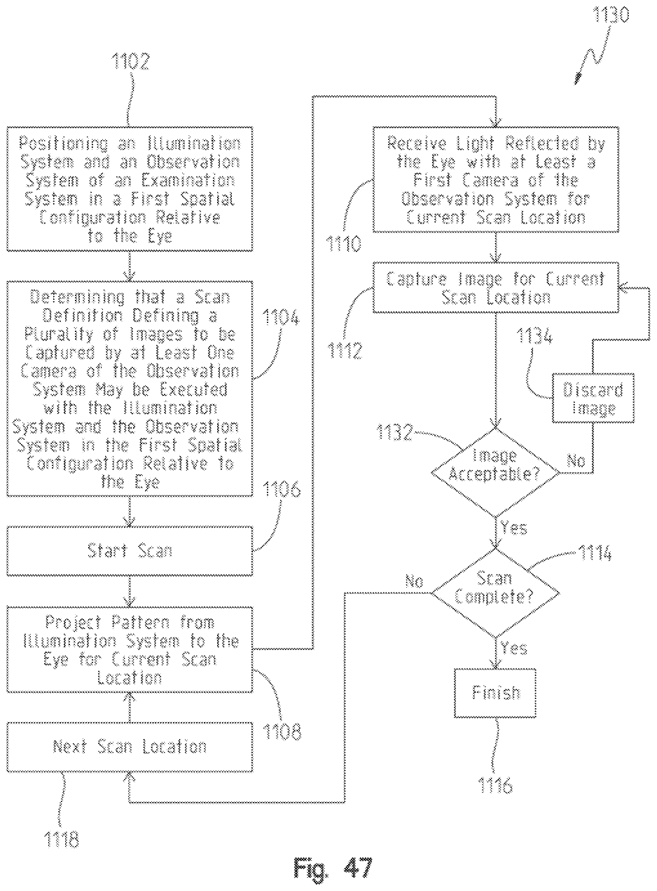

Referring to FIG. 47, another processing sequence 1130 of exam logic 320 is illustrated. Processing sequence 1130 is generally the same as processing sequence 1100, except that each captured image is reviewed by exam logic 320 to determine if the captured image is acceptable, as represented by block 1132. If so, processing sequence 1130 moves on to block 1114. If not, the image is discarded, as represented by block 1134, and processing sequence 1130 returns to block 1112 to recapture the image. In embodiments, exam logic 320 determines if the patient blinked during the capture of the image and, if so, determines the image is unacceptable in block 1132.

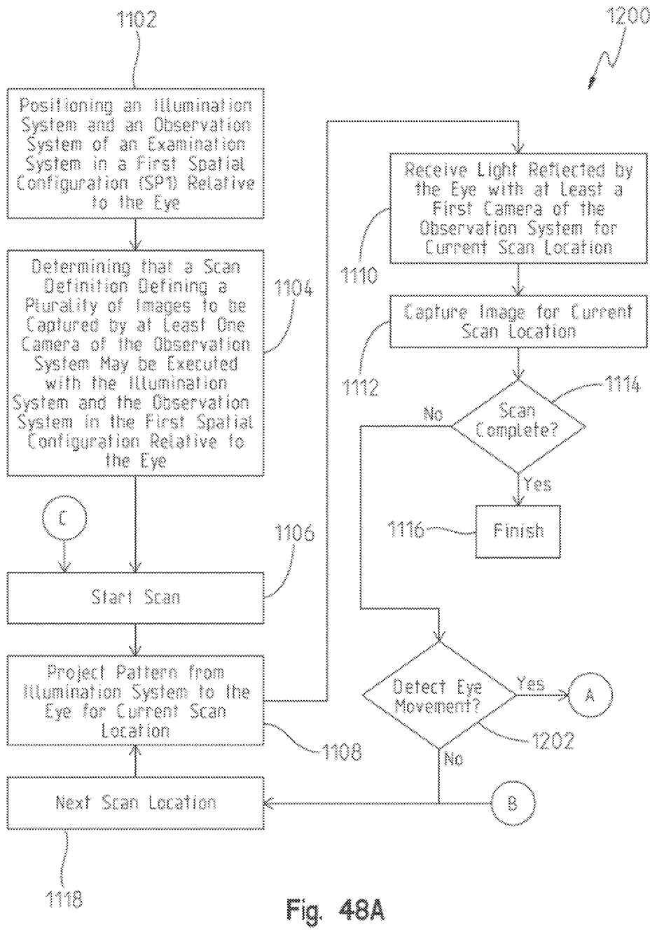

Referring to FIGS. 48A and 48B, another processing sequence 1200 of exam logic 320 is illustrated. Processing sequence 1200 is generally the same as processing sequence 1100, except that after each captured image, if the examination scan is not complete, exam logic 320 determines if there has been a movement of the eye as represented by block 1202. If not, the examination scan continues as in processing sequence 1100. If exam logic 320 determines eye 10 has moved, exam logic 320 next determines if the next scan position may be performed while illumination system 102 and observation system 104 are in the first spatial configuration, as represented by block 1204. If so, exam logic 320 adjust at least one characteristic of illumination system 102 and/or observation system 104 based on the determined eye movement, as represented by block 1206. In embodiments, a focal distance of illumination system 102 and observation system 104 are adjusted. In embodiments, a position of the pattern projected by illumination system 102 is adjusted. In embodiments, a focal distance of illumination system 102 and observation system 104 are adjusted and a position of the pattern projected by illumination system 102 is adjusted. Processing sequence 1200 then returns to block 1118.

If exam logic 320 determines the next scan position may not be performed while illumination system 102 and observation system 104 are in the first spatial configuration, as represented by block 1204, exam logic 320 determines if a second spatial configuration of illumination system 102 and observation system 104 may allow a remainder of scan definition 1150 to be executed, as represented by blocks 1208 and 1210. If so, exam logic 320 positions illumination system 102 and observation system 104 in the second spatial configuration relative to eye 10, as represented by block 1212, and control returns to block 1118. If not, an indicator is provided to the operator that the patient needs to be repositioned relative to examination system 100, as represented by block 1214. Exemplary indicators include visual indicators, audible indicator, tactile indicators, or combinations thereof. In one example, a message is displayed on a screen by controller 300.

Exam logic 320 waits to receive an input from the operator, as represented by blocks 1216, 1218. Once the operator repositions the patient relative to examination system 100, the operator may provide an input to resume the scan, as represented by block 1216, or to restart scan, as represented by block 1218. If the scan is to be resumed control returns to block 1208. If the scan is to be restarted control returns to block 1106. Exemplary inputs include a button press, a selection of a region on a display, a switch, or other suitable inputs.

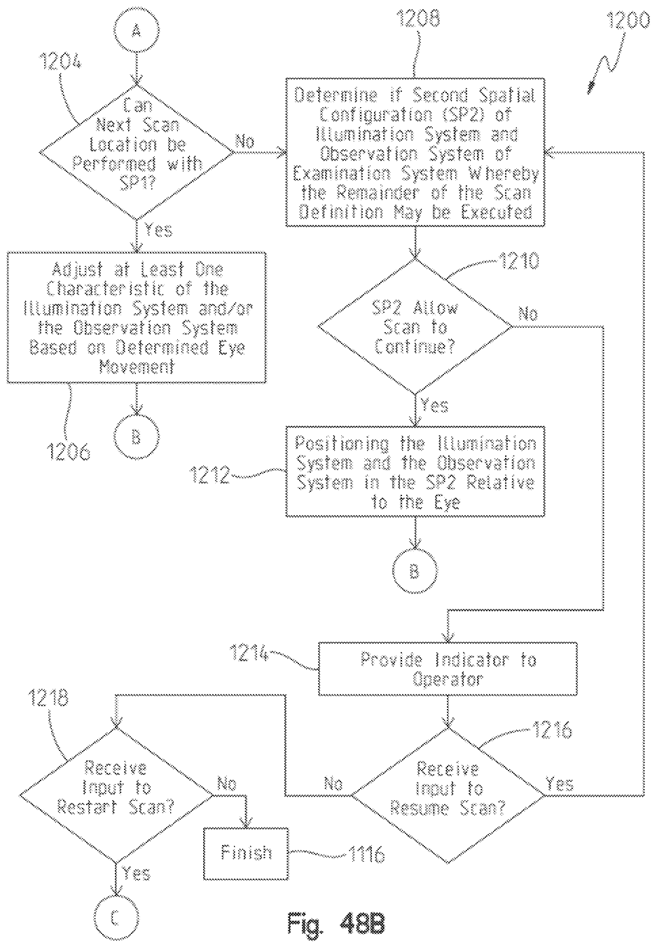

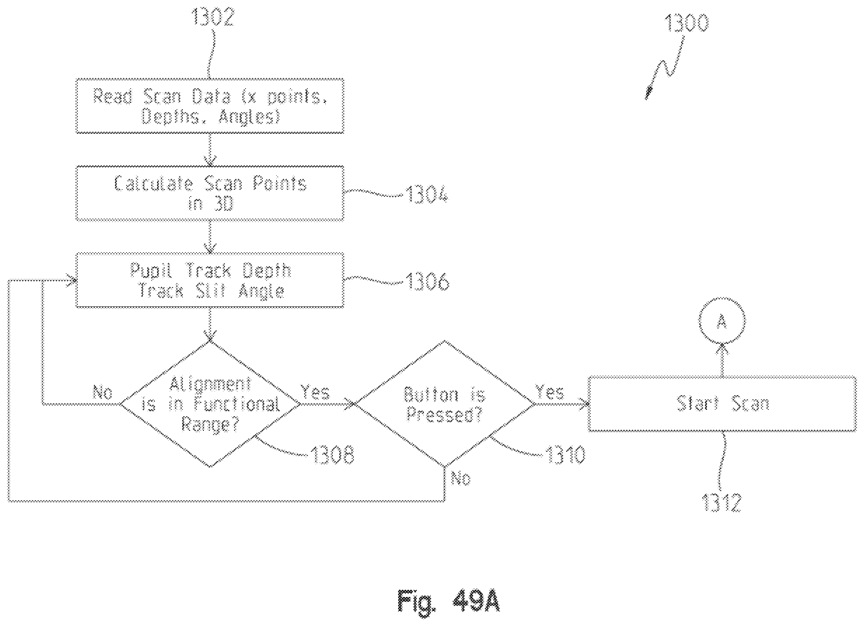

Referring to FIGS. 49A and 49B, another exemplary processing sequence 1300 for examination system 100 is shown. Exam logic 320 receives scan definition 1150, as represented by block 1302. The illustrated scan definition 1150 incudes traverse points along the x-axis (see FIGS. 27 and 27A) for location of a pattern with illumination system 102. The traverse points may be a location of a vertical slit or a location of a first vertical slit of a plurality of vertical slits. The traverse points may be a location for a registration point in an image projected by illumination system 102 that includes a pattern. The pattern may be one or more vertical slits. The illustrated scan definition 1150 further includes focal depths in the z-direction (see FIGS. 27 and 27A). The illustrated scan definition 1150 may further include angles of illumination system 102 and observation system 104. For example, observation system 104 may be straight on relative to eye 10 (0 degree angle) and illumination system 102 may be rotated 30 degrees to the left of observation system 104.

Exam logic 320 as described herein determines the scan points (such as point 1154 and point 1156) that correspond to the received scan data in block 1302, as represented by block 1304. Exam logic 320 next or continues to track a transverse position of eye 10 relative to examination system 100, a depth position of eye 10 relative to examination system 100, and the current angles of illumination system 102 and observation system 104, as represented by block 1306. As discussed herein, exam logic 320 is able to account for any initial misalignments of examination system 100 relative to eye 10 and/or movements of eye 10 relative to examination system 100 subsequent to an initial alignment of examination system 100 to eye 10.

Exam logic 320 determines if the requested scan may be performed by examination system 100 and the current positioning of eye 10 relative to examination system 100, as represented by block 1308. For example, as described herein, exam logic 320 by knowing scan envelope 1152 of examination system 100 is able to determine if each requested scan location (determined in step 1304) may be captured with examination system 100 and the current spatial configuration of illumination system 102 and observation system 104. If exam logic 320 determines that the requested scan may not be performed with the current positioning of eye 10 relative to examination system 100, control is returned to block 1306 to continue to track the positioning of eye 10 relative to examination system 100. In embodiments, exam logic 320 may provide an indication to the operator that the requested scan may not be performed such that the operator may manually adjust the current positioning of eye 10 relative to examination system 100. In embodiments, exam logic 320 may move illumination system 102 and observation system 104 to a second spatial configuration relative to eye 10, such as with a movement of moveable base 140, and reevaluate the ability of examination system 100 to perform the requested scan.

If examination system 100 is able to perform the requested scan, exam logic 320 waits for an operator input, as represented by block 1310, and once received starts the scan, as represented by block 1312. Although a button push is shown as the exemplary input other suitable inputs, such as those disclosed herein, may be used. In embodiments, exam logic 320 does not wait for an operator input. Rather, once it is determined that examination system 100 is able to perform the requested scan, exam logic 320 begins the scan without further input from the operator.

Referring to FIG. 49B, exam logic 320 prepares to capture one or more images for a current scan location, as represented by block 1314. Exam logic continues to track the transverse position of eye 10 relative to examination system 100, the depth position of eye 10 relative to examination system 100, and the angles of illumination system 102 and observation system 104, as represented by block 1316. Exam logic 320 determines if the track data from block 1316 is valid, as represented by block 1318. If not, exam logic 320 uses the last known track data, as represented by block 1320. Exemplary invalid data includes instances wherein pupil coordinates could not be determined by the camera used for tracking the pupil, such as first camera 502, due to one or more of poor illumination, an occurrence of a blink, and motion blur. In this situation, the depth camera(s), such as cameras 504 and 504′, would not know where in its field of view to look for the eye and the position of the eye 10 is not known in any of X, Y, or Z directions. Additional exemplary invalid data includes instances wherein the pupil is located by the camera used for tracking the pupil, such as first camera 502, but the depth camera(s), such as cameras 504 and 504′, provide data that results in an unrealistic depth value due to one or more of noise, artifacts, and other events. Exemplary unrealistic values include zero or infinity which are disregarded by exam logic 320. In this situation, the position of the eye 10 is known in X and Y directions, but not in the Z or depth direction.

If the track data from block 1316 is valid, exam logic 320 determines if eye 10 is within the range limits of examination system 100, as represented by block 1322. If eye 10 is not within the range limits of examination system 100, such as the current scan location is not within scan envelope 1152 of examination system 100, exam logic 320 returns to block 1316. Once exam logic 320 either decides that eye 10 is within the range limits of examination system 100 or decides to use the last known track data, exam logic 320 determines the transverse value (xᵢ′), the focal distance (fₛ′), and the focal distance (fᵢ′) (see FIG. 27A), as represented by block 1324. Exam logic 320 next focuses illumination system 102 at the appropriate focal depth, focuses observation system 104 at the appropriate focal depth, and updates a location of the pattern projected by illumination system 102, as represented by block 1326. Additionally, in some embodiments, exam logic 320 also updates the angular positions of one or more of illumination system 102 and observation system 104 as called for by the requested scan.

Exam logic 320 captures one or more images at each scan location with one or more cameras of observation system 104, as represented by block 1328. Exam logic next determines if additional scan points need to be captured, as represented by block 1330. If so, control returns to block 1314. If not, the data and images captured during the scan are saved to associated memory 312, as represented by block 1332.

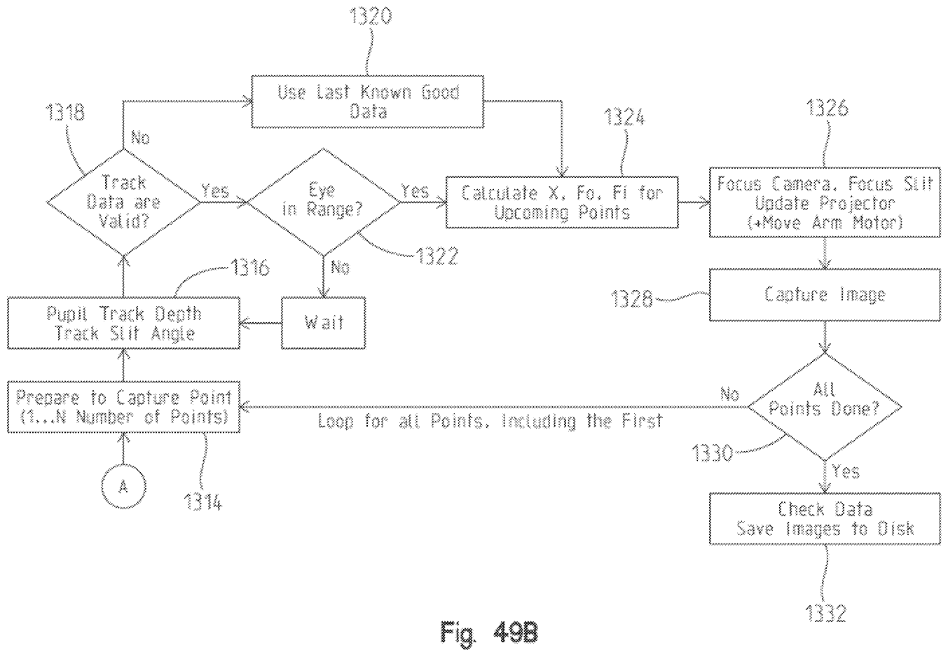

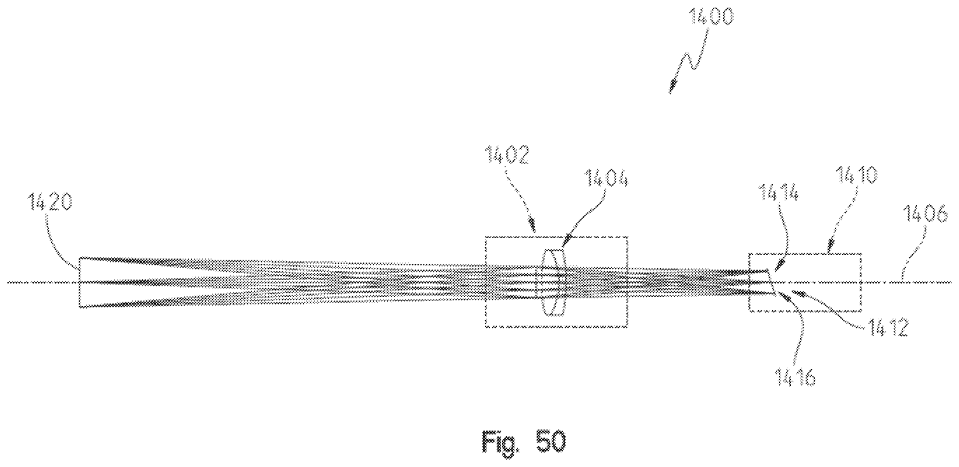

As mentioned herein transverse tracking of eye 10 relative to examination system 100 may be accomplished by tracking a limbus of eye 10 or other feature, such as the pupil, and other suitable methodologies and setups. Further, as mentioned herein depth tracking of eye 10 relative to examination system 100 may be accomplished by stereo cameras, alignment of a test pattern and other suitable methodologies. FIGS. 50-56 detail additional methodologies and systems that may be used for one or more of transverse tracking of eye 10 relative to examination system 100 and depth tracking of eye 10 relative to examination system 100.

Referring to FIG. 50, examination system 100 may further include a system 1400 to determine a separation between an object, such as eye 10, and a medical device, such as examination system 100. System 1400 includes an optical assembly 1402 including at least one lens 1404 and a camera 1410. Optical assembly 1402 defines an optical axis 1406. Camera 1410 includes an image sensor 1412, such as an array of detectors which form a rectilinear image of an object plane 1420. In embodiments, optical assembly 1402 is part of camera 1410.

As shown in FIG. 50, object plane 1420 is orthogonal to optical axis 1406 while image sensor 1412 is tilted relative to optical axis 1406. In the illustrated embodiment, image sensor 1412 is tilted relative to optical axis 1406 of optical system 1402 such that a first portion 1414 of image sensor 1412 on a first side of a plane passing through the optical axis 1406 (in the illustrated embodiment the plane is through optical axis 1406 and projecting out of FIG. 50) and a second portion 1416 of image sensor 1412 on a second side of the plane passing through optical axis 1406. The second side of the plane being opposite the first side of the plane. Although shown as straight optical axis 1406 from object plane 1420 to image sensor 1412, optical assembly 1402 may have multiple bends, such as due to the inclusion of one or more mirrors and prisms as part of optical assembly 1402.

Controller 300 is configured to receive image data of the detected image from image sensor 1412 and to determine the separation between eye 10 and examination system 100 based on analyzing multiple portions of image data from image sensor 1412 including at least one of a first portion of the image data from first portion 1414 of image sensor 1412 and a second portion of image data from second portion 1416 of image sensor 1412. In embodiments, controller 300 further analyzes at least a third portion of the image data. The third portion of image data being closer to the plane than the at least one of the first portion of the image data from first portion 1414 of image sensor 1412 and the second portion of the image data from second portion 1416 of image sensor 1412. In embodiments, the third portion of image data is on both sides of the plane. In embodiments, controller 300 analyzes both the first portion of the image data from first portion 1414 of image sensor 1412 and the second portion of the image data from second portion 1416 of image sensor 1412.

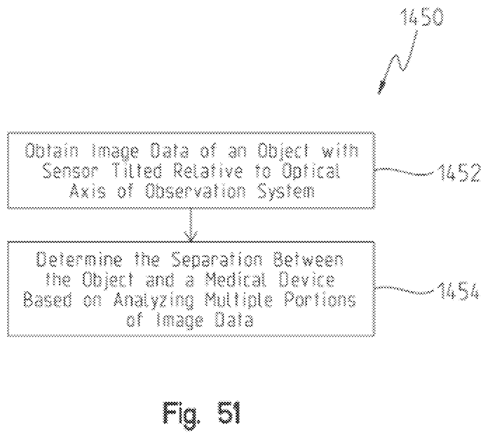

Referring to FIG. 51, an exemplary processing sequence 1450 of exam logic 320 is shown. Image date is obtained with image sensor 1412 which is tilted relative to optical axis 1406 of system 1400 which may be part of observation system 104, as represented by block 1452. Exam logic 320 determines the separation between eye 10 and examination system 100 based on analyzing multiple portions of image data from image sensor 1412, as represented by block 1454.

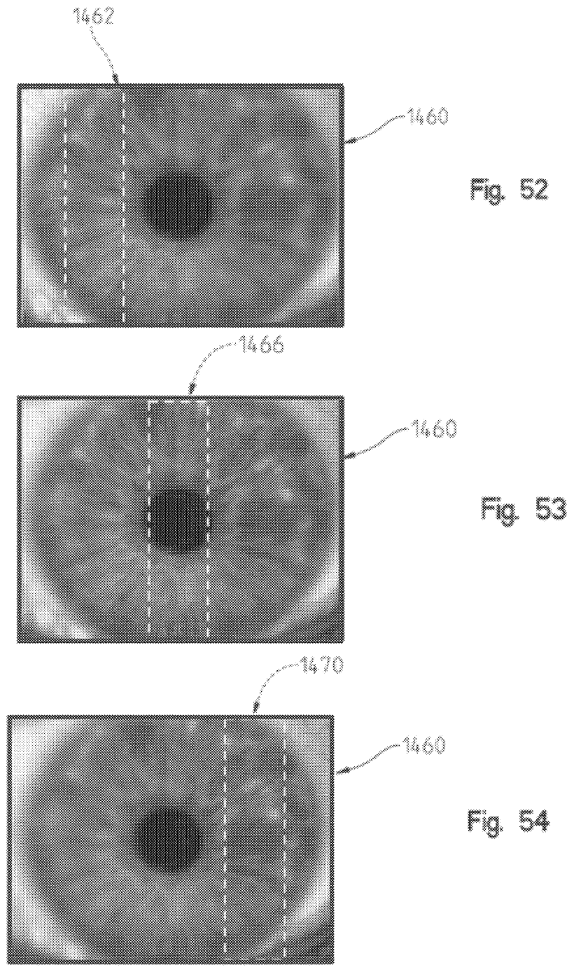

Referring to FIG. 52, an image 1460 of eye 10 captured by image sensor 1412 is shown. Image 1460 has an in focus portion 1462 on the left side of eye 10 which corresponds to first portion 1414 of image sensor 1412. When portion 1462 is in focus eye 10 is too close to examination system 100. The remainder of image 1460 is out of focus.

Referring to FIG. 53, an image 1464 of eye 10 captured by image sensor 1412 is shown. Image 1464 has an in focus portion 1466 in the center of eye 10 which corresponds to an on axis portion of image sensor 1412. When the on axis portion is in focus eye 10 is properly spaced relative to examination system 100. The remainder of image 1464 is out of focus.

Referring to FIG. 54, an image 1468 of eye 10 captured by image sensor 1412 is shown. Image 1468 has an in focus portion 1470 on the right side of eye 10 which corresponds to second portion 1416 of image sensor 1412. When portion 1470 is in focus eye 10 is too far from examination system 100. The remainder of focus portion 1470 is out of focus.

In embodiments, exam logic 320 exams the sharpness levels of at least a portion of an image captured by image sensor 1412 to determine the position of examination system 100 relative to eye 10. By finding the portion of the captured image with the highest sharpness level, exam logic 320 may determine if examination system 100 is at the correct distance relative to eye 10, too far from eye 10, or too near to eye 10. An offset corresponding to the row or column of pixels having the highest sharpness from the row or column of pixels intersecting optical axis 1406 may be converted to a distance through empirical data stored in a look-up table or through a formulaic relationship. For example, exam logic 320 may determine which column of the image captured by image sensor 1142 has the highest sharpness level and based on the number of columns separating the column with the highest sharpness from a central column of the image sensor 1142, a distance may be determined from empirical data. When image sensor 1142 is tilted vertically (in and out of the page), instead of horizontally as shown in FIG. 50, exam logic 320 may determine which row of the image captured by image sensor 1142 has the highest sharpness level and based on the number of rows separating the row with the highest sharpness from a central row of the image sensor 1142, a distance may be determined from empirical data.

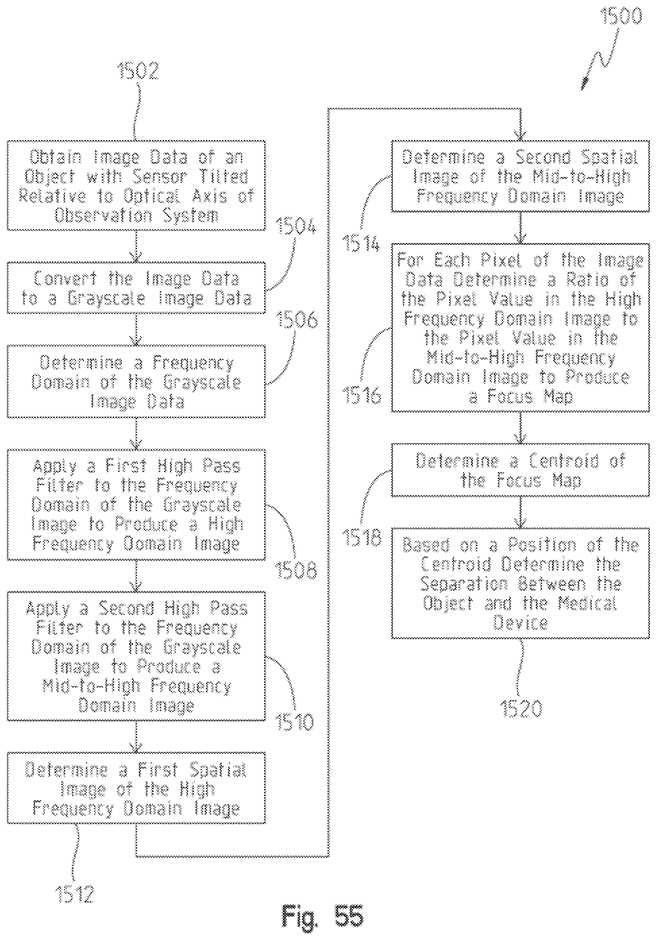

In embodiments, exam logic 320 analyzes a frequency component of the image data to determine the separation between eye 10 and examination system 100. The frequency component may be a frequency domain component. Referring to FIG. 55, an exemplary processing sequence 1500 for determining the separation between examination system 100 and eye 10 based on sharpness is shown. Exam software receives image data from image sensor 1412, as represented by block 1502. If not already a grayscale image, the image data is converted to a grayscale image, as represented by block 1504. In embodiments, exam logic 320 crops or masks the image data to remove portions of the image not of interest. For example, if examination system 100 is to be focused on cornea 12 the portion of the image including the eyelid is not of interest and need not be analyzed while the portion of the image including the iris is of interest and should be analyzed. A frequency domain image of the grayscale image is determined, as represented by block 1506. In embodiments, the frequency domain image is determined through a fast Fourier transform process.

A first high pass filter is applied to the frequency domain image of the grayscale image to produce a high frequency domain image, as represented by block 1508. A second high pass filter is applied to the frequency domain image of the grayscale image to produce a mid-to-high frequency domain image, as represented by block 1510. The second high pass filter has a lower frequency threshold than the first high pass filter and each may be set based on the expected image complexity of the object being viewed. For example, the iris of the eye has more high frequency components than many other anatomical structures. Thus, when imaging the iris of the eye each threshold of the first high pass filter and the second high pass filter may be set higher than when imaging other anatomical structures. In general, the second high pass filter removes a DC component from the image and low frequencies while the first high pass filter removes the DC component and low and mid frequencies to provide only the high frequency content. In embodiments, exam logic 320 may exam the frequency range of the frequency domain image and sets each of the first high pass filter threshold and the second high pass filter threshold as a percentage of the overall frequency range.

The high frequency domain image is converted to a first spatial image, as represented by block 1512. The mid-to-high frequency domain image is converted to a second spatial image, as represented by block 1514. In embodiments, the edges of each of the first spatial image and the second spatial image are masked or otherwise set to a constant value to avoid artifacts introduced into the images. Next, exam logic 320 for each pixel of the image data determines a ratio of the pixel value in the first spatial image to the pixel value in the second spatial image to produce a focus map, as represented by block 1516. A centroid of the focus map is determined, as represented by block 1518. Based on a position of the centroid exam logic 320 determines the separation between the object and the medical device is determined, as represented by block 1520. In embodiments, an offset of the pixel corresponding to the centroid from the row or column of pixels intersecting optical axis 1406 may be converted to a distance through empirical data stored in a look-up table or through a formulaic relationship.

In embodiments, exam logic 320 may use camera 1410 to track transverse movement of eye 10 as well. This may be accomplished by tracking a location of the pupil of eye 10 in the captured image and/or a location of the limbus.

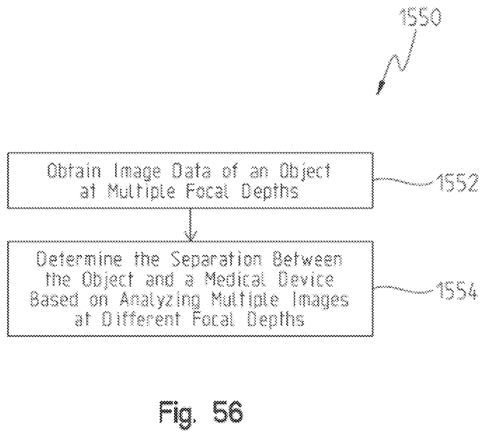

Referring to FIG. 56, an exemplary processing sequence 1550 of exam logic 320 is shown. Image date is obtained with first camera 502 at multiple focuses of tunable lens 524, as represented by block 1552. Exam logic 320 determines the separation between eye 10 and examination system 100 based on analyzing the images obtained at multiple focal depths from first camera 502, as represented by block 1554.

In embodiments, exam logic 320 analyzes a frequency component of each of the images to determine which image corresponds to examination system 100 being at the correct separation from eye 10. In examples, the frequency component may be a frequency domain component.

In embodiments, the image corresponding to the highest sharpness in a portion that intersects axis 506 is the image corresponding to examination system 100 being at the correct separation from eye 10. Based on the focal length of tunable lens 520, exam logic 320 can determine a current separation of examination system 100 from eye 10 and, if desired, exam logic 320 may move examination system 100 relative to eye 10 to alter the current separation of examination system 100 from eye 10.

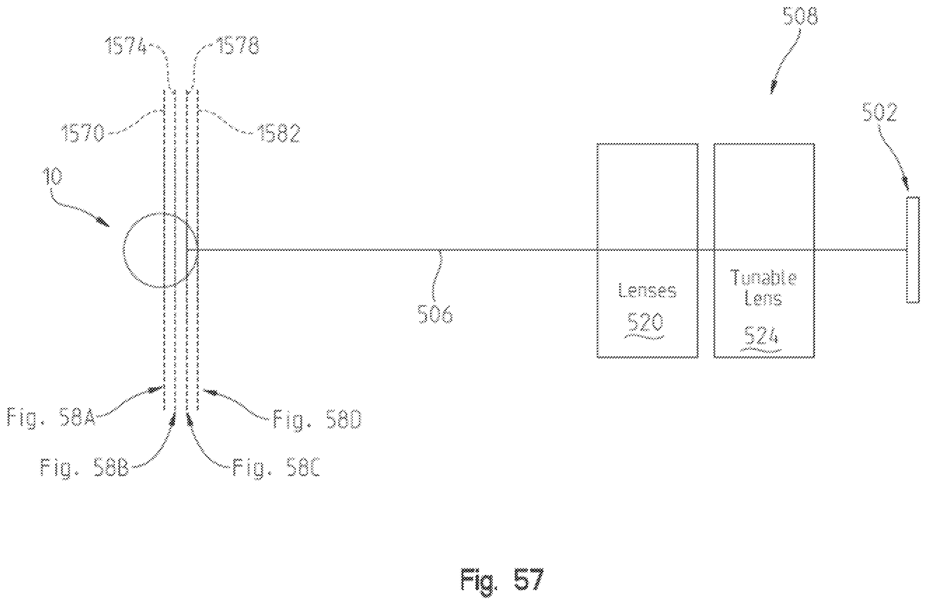

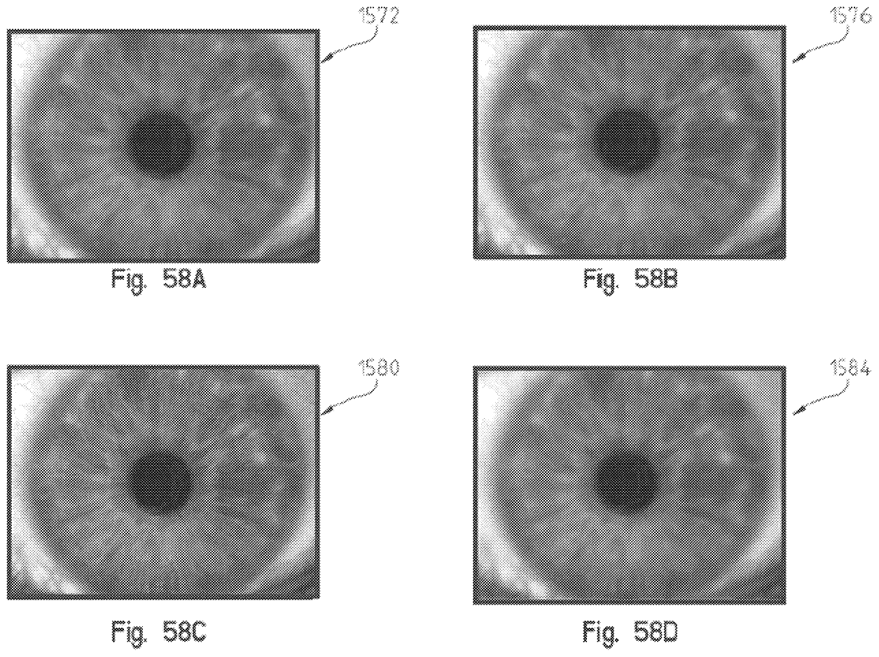

Referring to FIG. 57, first camera 502 and optics 508 are shown relative to eye 10. Image date is obtained with first camera 502 at multiple focuses of tunable lens 524. Four exemplary focal planes corresponding to different configurations of tunable lens 524 are shown. Focal plane 1570 corresponds to image 1572 of eye 10 shown in FIG. 58A. Focal plane 1574 corresponds to image 1576 of eye 10 shown in FIG. 58B. Focal plane 1578 corresponds to image 1580 of eye 10 shown in FIG. 58C. Focal plane 1582 corresponds to image 1584 of eye 10 shown in FIG. 58D. Additional or fewer focal planes may be imaged.

As shown in FIG. 58C, iris 16 of eye 10 is more in focus than in FIGS. 58A, 58B, or 58D. Further, iris 16 of eye 10 has a complex intensity profile as opposed to the sclera of eye 10 which is generally more uniform. Thus, iris 16 of eye 10 provides a quality target for controller 300 to analyze for focus analysis based on the sharpness of the images received by first camera 502.

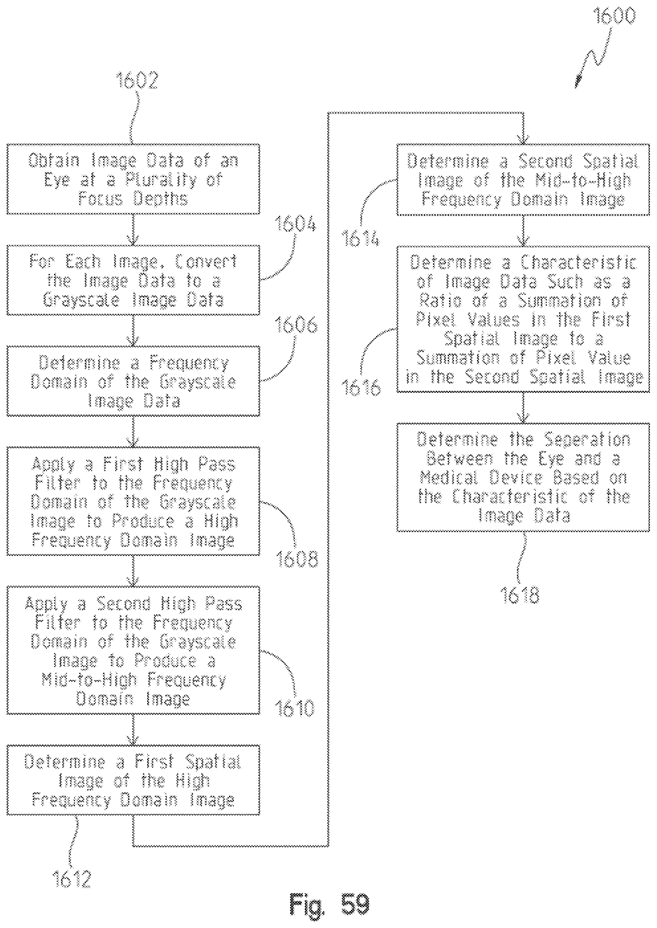

Referring to FIG. 59, an exemplary processing sequence 1600 for determining the separation between examination system 100 and eye 10 based on sharpness is shown. Exam software 320 receives image data from camera 502 at a plurality of focal depths, as represented by block 1602. The image data may be at least one image for each focal depth resulting in a plurality of images. Each focal depth corresponds to a characteristic of tunable lens 524. Tunable lens 524 is one example of how an optical system may be an adjustable optical system. In other embodiments, an adjustable optical system may include one or more movable lens in place of or in addition to one or more tunable lens. In embodiments, the plurality of focal depths are for parallel planes while first camera 502 is positioned directly in front of eye 10, such as shown in FIG. 57.

Returning to FIG. 59, If not already a grayscale image, the image data of each image of the plurality of images is converted to a grayscale image, as represented by block 1604. In embodiments, exam logic 320 crops or masks the image data to remove portions of the image not of interest. For example, if examination system 100 is to be focused on cornea 12 the portion of the image including the eyelid is not of interest and need not be analyzed while the portion of the image including the iris 16 is of interest and should be analyzed. A frequency domain image of the grayscale image is determined, as represented by block 1606. In embodiments, the frequency domain image is determined through a fast Fourier transform process.

A first high pass filter is applied to the frequency domain image of the grayscale image to produce a high frequency domain image, as represented by block 1608. A second high pass filter is applied to the frequency domain image of the grayscale image to produce a mid-to-high frequency domain image, as represented by block 1610. The second high pass filter has a lower frequency threshold than the first high pass filter and each may be set based on the expected image complexity of the object being viewed. For example, the iris 16 of the eye 10 has more high frequency components than many other anatomical structures, such as the sclera of the eye 10. Thus, when imaging the iris 16 of the eye 10 each threshold of the first high pass filter and the second high pass filter may be set higher than when imaging other anatomical structures. In general, the second high pass filter removes a DC component from the image data and low frequencies while the first high pass filter removes the DC component and low and mid frequencies to provide only the high frequency content of the image data. In embodiments, exam logic 320 may exam the frequency range of the frequency domain image and sets each of the first high pass filter threshold and the second high pass filter threshold as a percentage of the overall frequency range.

The high frequency domain image is converted to a first spatial image, as represented by block 1612. The mid-to-high frequency domain image is converted to a second spatial image, as represented by block 1614. In embodiments, the edges of each of the first spatial image and the second spatial image are masked or otherwise set to a constant value to avoid artifacts introduced into the images. Next, exam logic 320 determines a characteristic of the image data, as represented by block 1616. Exemplary characteristics include a ratio of a summation of the pixel values in the first spatial image to a summation of pixel values in the second spatial image and other suitable characteristics. Based on the characteristic of the image data, exam logic 320 determines the separation between the eye 10 and examination system 100, as represented by block 1620. In embodiments, the image having an extreme value of the characteristic of the image data, such as the highest ratio in step 1616, is selected by exam logic 320. The image corresponding to the highest ratio will to the image having the highest amount of image detail that remained after the first high pass filter which is the image with the highest sharpness for a complex object like iris 16 of eye 10. The characteristic of tunable lens 524 for the selected image of the plurality of images, such as the focal length of tunable lens 524 for the captured image, may be converted to a distance separating eye 10 from examination system 100 through empirical data stored in a look-up table or through a formulaic relationship.

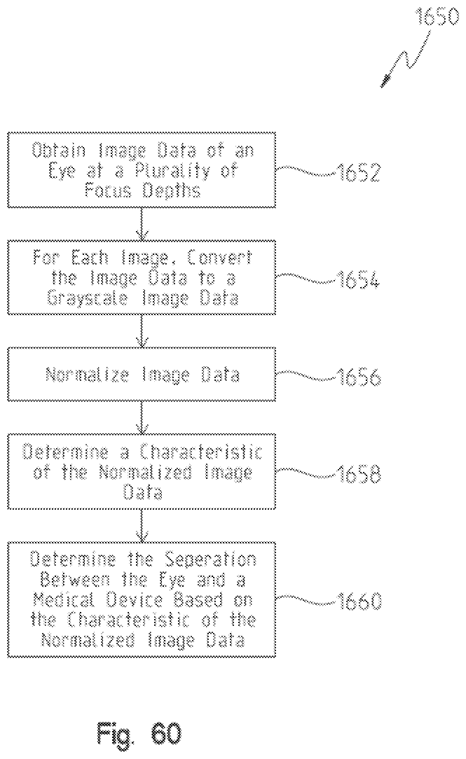

Referring to FIG. 60, another exemplary processing sequence 1650 for determining the separation between examination system 100 and eye 10 based on sharpness is shown. Exam software 320 receives image data from camera 502 at a plurality of focal depths, as represented by block 1652. The image data may be at least one image for each focal depth resulting in a plurality of images. Each focal depth corresponds to a characteristic of tunable lens 524. Tunable lens 524 is one example of how an optical system may be an adjustable optical system. In other embodiments, an adjustable optical system may include one or more movable lens in place of or in addition to one or more tunable lens. In embodiments, the plurality of focal depths are for parallel planes while first camera 502 is positioned directly in front of eye 10, such as shown in FIG. 57.

Returning to FIG. 60, If not already a grayscale image, the image data of each image of the plurality of images is converted to a grayscale image, as represented by block 1654. In embodiments, exam logic 320 crops or masks the image data to remove portions of the image not of interest. For example, if examination system 100 is to be focused on cornea 12 the portion of the image including the eyelid is not of interest and need not be analyzed while the portion of the image including the iris 16 is of interest and should be analyzed. The image data of the grayscale image is normalized, as represented by block 1656. Exam logic 320 determines a characteristic of each normalized image, as represented by block 1658. Based on the characteristic of the normalized image data, exam logic 320 determines the separation between the eye 10 and examination system 100, as represented by block 1660.

In embodiments, exam logic 320 determines a convolution of the normalized image data with an edge finding filter. Exemplary edge finding filters include a Sobel filter and a Prewitt filter. The pixel values of the resultant convolution image are summed by exam logic 320 to provide a characteristic of the normalized data. The image having an extreme value of the characteristic of the image data, such as the highest sum of the convolution image, is selected by exam logic 320. The image corresponding to the highest sum of the convolution image is the image having the highest amount of image detail, in particular edges which is the image with the highest sharpness for a complex object, like iris 16 of eye 10. The characteristic of tunable lens 524 for the selected image of the plurality of images, such as the focal length of tunable lens 524 for the captured image, may be converted to a distance separating eye 10 from examination system 100 through empirical data stored in a look-up table or through a formulaic relationship.

In embodiments, exam logic 320 determines a variance of the intensity of the pixel data of the normalized image data. The image having an extreme value of the characteristic of the image data, such as the greatest variance of the pixel data of the normalized image data, is selected by exam logic 320. The image corresponding to the greatest variance of the pixel data of the normalized image data is the image having the highest amount of image detail which is the image with the highest sharpness for a complex object, like iris 16 of eye 10. The characteristic of tunable lens 524 for the selected image of the plurality of images, such as the focal length of tunable lens 524 for the captured image, may be converted to a distance separating eye 10 from examination system 100 through empirical data stored in a look-up table or through a formulaic relationship.

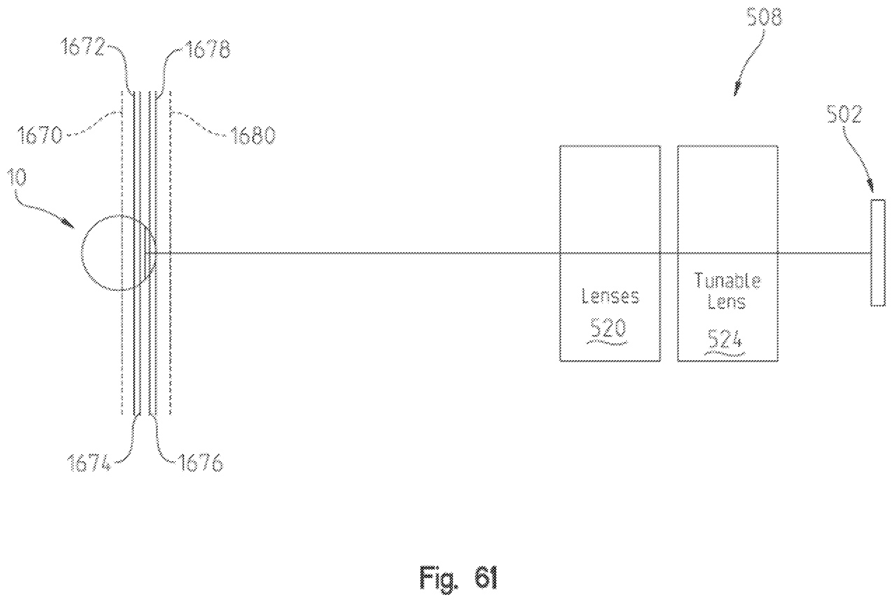

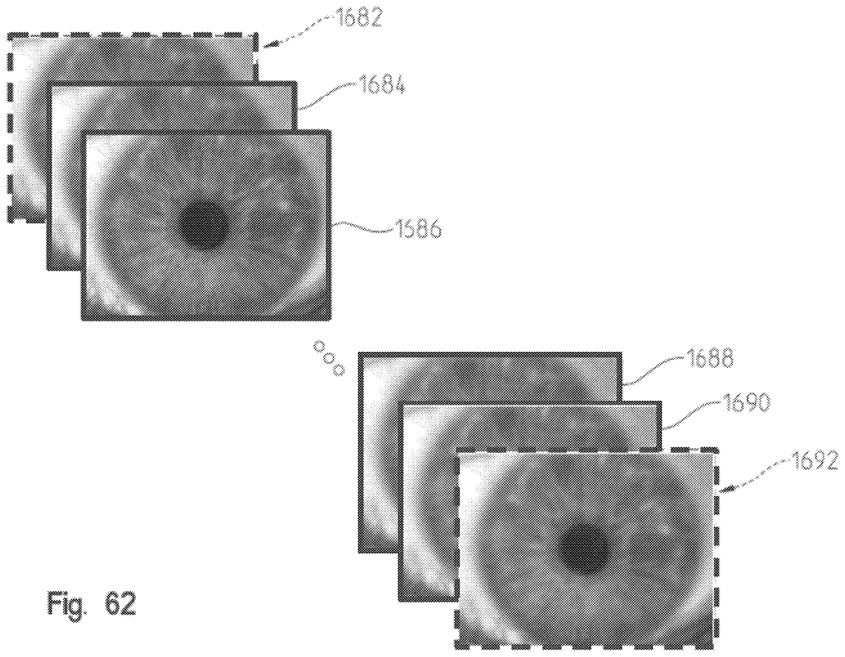

Referring to FIG. 61, first camera 502 and optics 508 are shown relative to eye 10. Image date is obtained with first camera 502 at multiple focuses of tunable lens 524. Six exemplary focal planes corresponding to different configurations of tunable lens 524 are shown. Respective focal planes 1670-1680 correspond to images 1682-1692 in FIG. 62. Additional or fewer focal planes may be imaged. In embodiments, at least six images are captured at various focal lengths and at least a portion of the captured images may correspond to focal planes outside of typical scan depths in the scan definitions of examination system 100.

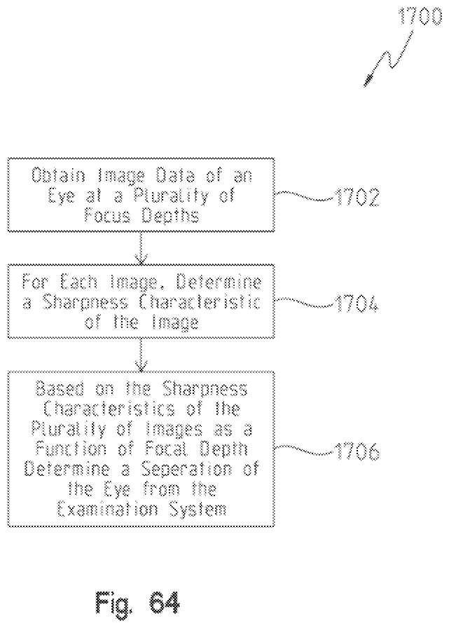

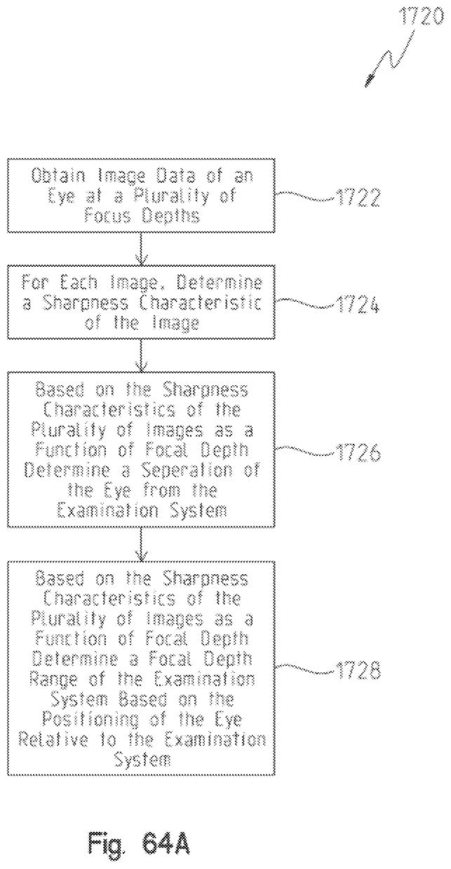

An exemplary processing sequence 1700 for determining at least the separation between examination system 100 and eye 10 based on the images in FIG. 62 is shown in FIG. 64. Exam logic 320 obtains image data of eye 10 at a plurality of focal depths, such as shown in FIGS. 61 and 62, as represented by block 1702. For each image, exam logic 320 determines a sharpness characteristic of the image, as represented by block 1704. Based on the determined sharpness characteristics, exam logic 320 determines a separation between eye 10 and examination system 100, as represented by block 1706.

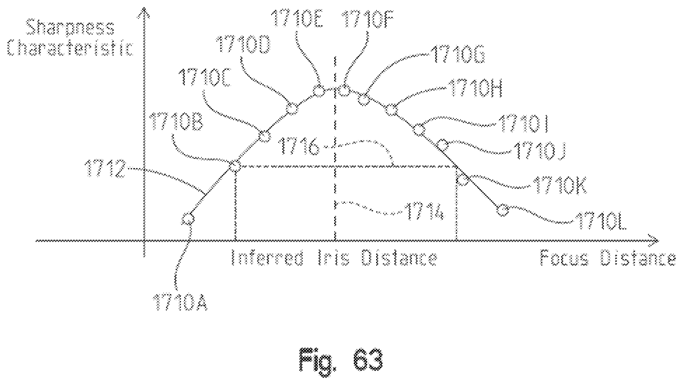

In embodiments, exam logic 320 fits a curve 1712 to a data set of focal length of tunable lens 524 and hence the overall focal length of optics 508 and sharpness characteristic shown as data points 1710A-1710L in FIG. 63. Twelve data points are shown in FIG. 63 corresponding to twelve images captured by first camera 502 (as opposed to the six shown in FIGS. 61 and 62). Curve 1712 may be a bell function or a parabola. An apex of curve 1712, designated by vertical line 1714, corresponds to the focal length of tunable lens 524 and hence the overall focal length of optics 508 that provides an image with the highest sharpness. This focal length may be used by exam logic 320 in block 1706 to determine a distance to eye 10 based on stored empirical data or a function.

Referring to FIG. 64A, an exemplary processing sequence 1720 for determining at least the separation between examination system 100 and eye 10 based on captured images, such as the images in FIG. 62, is shown. Exam logic 320 obtains image data of eye 10 at a plurality of focal depths, such as shown in FIGS. 61 and 62, as represented by block 1702. For each image, exam logic 320 determines a sharpness characteristic of the image, as represented by block 1704. Based on the determined sharpness characteristics, exam logic 320 determines a separation between eye 10 and examination system 100, as represented by block 1706. An exemplary method of determining the separation between eye 10 and examination system 100 is discussed above in relation to FIG. 63.

Returning to FIG. 64A, exam logic 320 further determines based on the sharpness characteristics of the plurality of images a focal depth range of examination system 100 based on a current positioning of eye 10 relative to examination system 100, as represented by block 1728. In embodiments, the focal depth range corresponds to focal depths satisfying a minimum level of sharpness. For example, in FIG. 63, an intersection of a line 1716 with curve 1712 represents the focal depths corresponding to a full width half-maximum of curve 1712. Other methods of identifying a minimum level of sharpness may be used. In FIG. 63, the images corresponding to sharpness levels 1710A, 1710K, and 1710L do not satisfy the minimum level of sharpness.

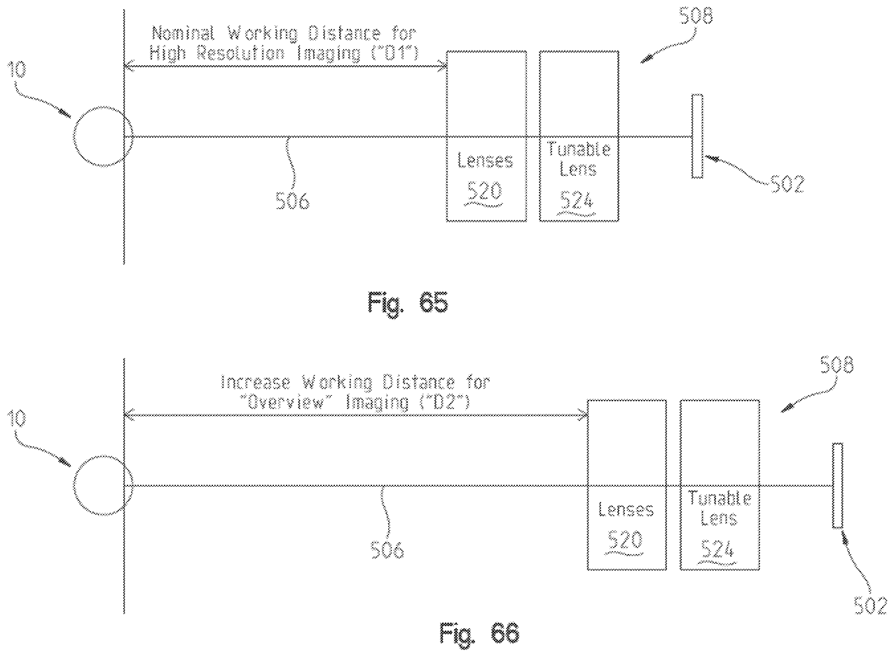

Referring to FIGS. 65 and 66, optics 508 and first camera 502 are shown positioned in front of eye 10 with axis 506 aligned relative to eye 10. In FIG. 65, optics 508 has a first separation (“D1”) from eye 10. In FIG. 66, optics 508 has a second separation (D2”) from eye 10, the second separation (“D2”) being greater than the first separation (“D1”). In embodiments, optics 508 and first camera 502 are movable relative to eye 10 through a movement of moveable base 140 relative to patient support 144. In both the position shown in FIG. 65 and the position shown in FIG. 66, examination system 100 is capable of obtaining images with first camera 502 at a plurality of focal depths by adjusting a characteristic of tunable lenses 524. While in the position shown in FIG. 65, examination system 100 may obtain several high-resolution images at different focal depths, each often depicting only a portion of eye 10 as described herein. While in the position shown in FIG. 66, examination system 100 may obtain several overview images at different focal depths, each showing the complete extent of eye 10.

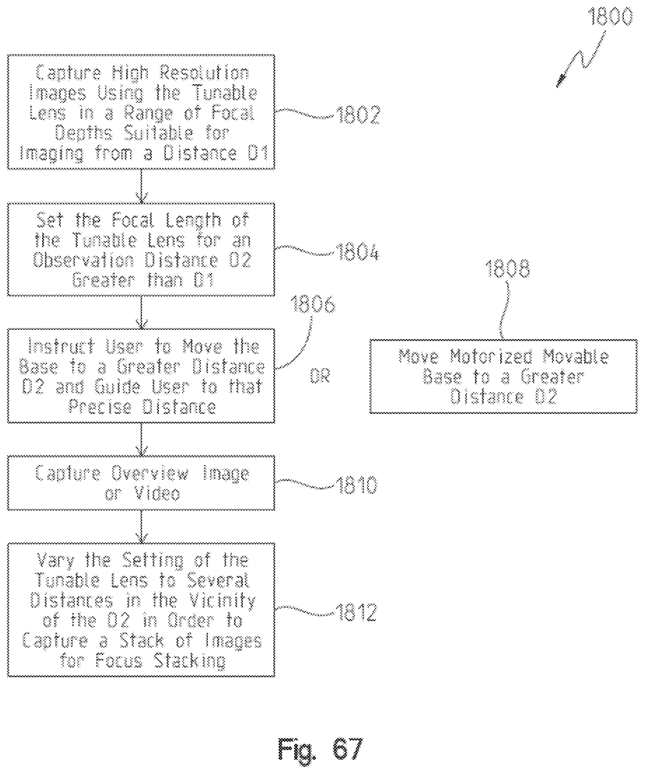

Referring to FIG. 67, an exemplary processing sequence 1800 of exam logic 320 is shown. Examination system 100 captures a plurality of images at different focal depths with second camera 504 and the initial separation of D1 in FIG. 65 by adjusting tunable lenses 524, as represented by block 1802. Tunable lens 524 is set to a second set of focal lengths more suitable for the separation D2 shown in FIG. 66 than D1 in FIG. 65, as represented by block 1804.

With tunable lenses 524 at a first focal length of the second set of focal lengths, exam logic 320 provides an instruction to an operator to move moveable base 140 relative to patient support 144, as represented by block 1806. Exemplary instructions may be visual or audio. An operator may use the method described in FIG. 10 or exam logic 320 may determine proper positioning with one or more of the methods disclosed herein and provide an indication to the operator when examination system 100 is in the correct position. Exemplary indications may be visual or audio. Alternatively, if moveable base 140 is controlled with controller 300, exam logic 320 may automatically move moveable base 140 to the correct position, as represented by block 1808.

With examination system 100 being in the arrangement of FIG. 66, first camera 502 captures an overview image or video of eye 10, as represented by block 1810. Further, tunable lenses 524 may be set to different focal lengths of the second set of focal lengths to obtain a series of images, as represented by block 1812.

In embodiments, examination system 100 provides a test pattern to be projected on eye 10 having a background intensity and at least two regions with a plurality of higher intensities. The at least two regions may form a compound slit.

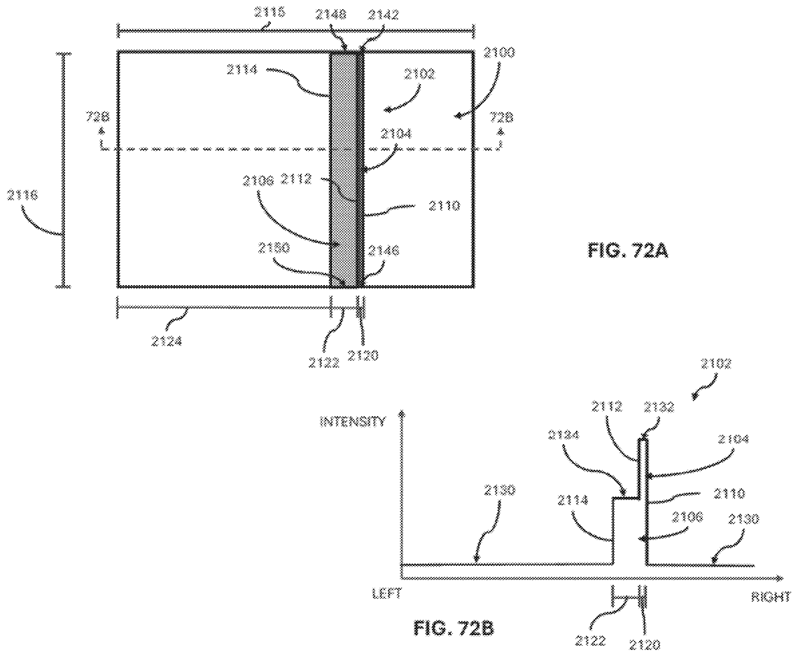

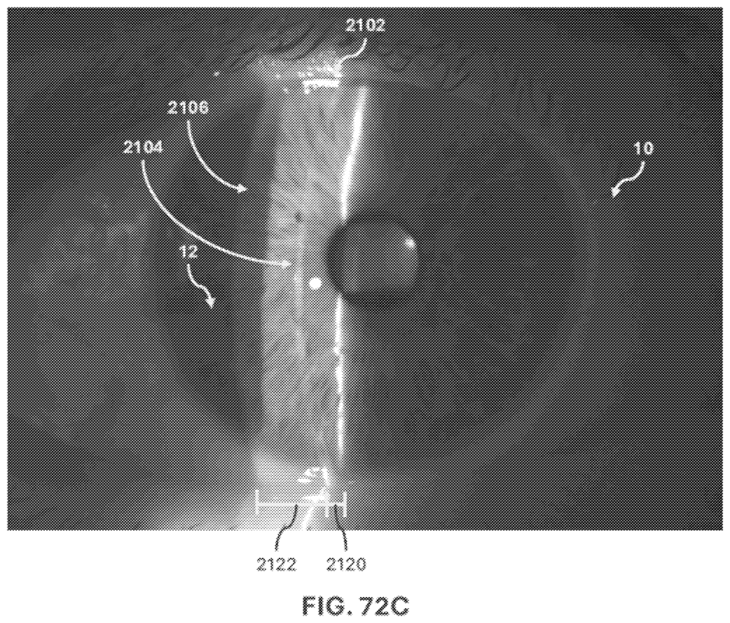

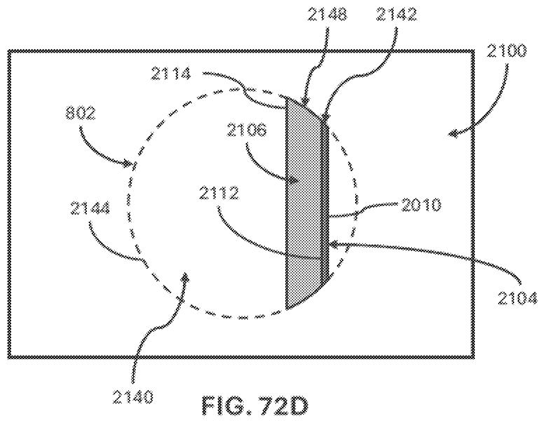

Referring to FIG. 72A, a first test pattern image 2100 of projector 402 is shown. First test pattern image 2100 includes an example of a first compound slit 2102. Compound slit 2102 includes a first region 2104 and a second region 2106. Each of first region 2104 and second region 2106 are rectilinear in shape. First region 2104 includes a first vertical edge 2110 and a second vertical edge 2112. Second region 2106 includes a first vertical edge 2114 and terminates at second vertical edge 2112 of first region 2104.

First region 2104 has a first width 2120 and extends a full height of the display area of projector 402. Second region 2106 has a second width 2122 and extends a full height of the display area of projector 402. The display area of projector 402 has an overall width 2115 and an overall height 2116. In embodiments, each of first region 2104 and second region 2106 may extend less than a full height of the display area of projector 402. In embodiments, each of first region 2104 and second region 2106 may extend generally a full height of the display area of projector 402. First region 2104 has an upper extent 2142 and a lower extent 2146. Similarly, controller 300 has an upper extent 2148 and a lower extent 2150. Edge 2114 of second region 2106 is offset from a leftside edge 2118 of the display area of projector 402 by an offset distance 2124.

As shown in FIG. 72A, an area of second region 2106 is larger than an area of first region 2104. In embodiments, second width 2122 of second region 2106 is greater than first width 2120 of first region 2104. In embodiments, second width 2122 of second region 2106 is more than twice first width 2120 of first region 2104. In embodiments, second width 2122 of second region 2106 is more than thrice first width 2120 of first region 2104. In embodiments, second width 2122 of second region 2106 is more than five times first width 2120 of first region 2104. In embodiments, second width 2122 of second region 2106 is more than ten times first width 2120 of first region 2104. In embodiments, second width 2122 of second region 2106 is more than twenty-five times first width 2120 of first region 2104. In embodiments, second width 2122 of second region 2106 is more than forty times first width 2120 of first region 2104. In embodiments, second width 2122 of second region 2106 is up to fifty times first width 2120 of first region 2104. In embodiments, second width 2122 of second region 2106 is in the range of about 0.5 millimeters to about 3.0 millimeters. In embodiments, first width 2120 of first region 2104 is in the range of about 0.04 millimeters to about 0.8 millimeters. In embodiments, an overall width of first region 2104 and second region 2106 is in the range of about 0.54 millimeters to about 3.8 millimeters. In embodiments, an overall width of first region 2104 and second region 2106 is in the range of about 1.5 millimeters to about 2.5 millimeters.

Referring to FIG. 72B, an intensity plot along lines 72B-72B in FIG. 72A is shown. The display area of projector 402 produces a background intensity 2130 in arear outside of first region 2104 and second region 2106. In embodiments, background intensity 2130 is zero. In embodiments, background intensity 2130 is a low level intensity. First region 2104 has a first intensity level 2132 across its width 2120 while second region 2106 has a second intensity level 2134 across its width 2122. Second intensity level 2134 of second region 2106 is less than first intensity level 2132 of first region 2104. Both of second intensity level 2134 of second region 2106 and first intensity level 2132 of first region 2104 are greater than background intensity 2130. 70. In embodiments, second intensity level 2134 of second region 2106 is less than 75 percent of first intensity level 2132 of first region 2104. In embodiments, second intensity level 2134 of second region 2106 is less than 50 percent of first intensity level 2132 of first region 2104. In embodiments, second intensity level 2134 of second region 2106 is less than 30 percent of first intensity level 2132 of first region 2104. In embodiments, second intensity level 2134 is at least ten times greater than background intensity 2130.

In embodiments, first region 2104 and second region 2106 each have an intensity profile that is uniform. In embodiments, first region 2104 and second region 2106 each have an intensity profile that is non-uniform along a height direction. In one example, a central position along a height of the respective first region 2104 and second region 2106 may have a first intensity value and a position closer to the respective upper extent 2142, upper extent 2148 and/or a position closer to the respective lower extent 2146, lower extent 2150 may have a second intensity value, the second intensity value being lower than the first intensity value. For example, a central portion of each of first region 2104 and second region 2106 may have a generally constant intensity value for a first height extent, such as 80 percent, a declining intensity value down to background intensity 2130 at a second height extent between the first height extent and each of upper extent 2142 and upper extent 2148, and a declining intensity value down to background intensity 2130 at a third height extent between the first height extent and each of lower extent 2146 and lower extent 2150.

Referring to FIG. 72C, first compound slit 2102 is shown projected onto eye 10. As shown in FIG. 72C, first region 2104 of first compound slit 2102 is focused by examination system 100 on cornea 12 of eye 10. In embodiments, for a requested focal depth controller 300 is configured to focus first region 2104 of first compound slit 2102 at the first focal depth. Second region 2106 may or may not be in focus depending on the width of second region 2106. Second region 2106 illuminates a broad area of cornea 12 and iris 16 while first region 2104 functions like a traditional slit of a slit lamp microscope. An advantage, among others, of first compound slit 2102 is the greater illumination of eye 10 while not washing out the images or impeding the examination of the eye with first region 2104. This allows more data to be captured about the eye in a given image, thus also likely leading to shorter examination times and reduced number of images needed to be stored to document an examination.

In embodiments, first compound slit 2102 is produced by projector 402 of illumination system 102 and the image of FIG. 72C is captured by observation system 104. As discussed herein, controller 300 of examination system 100 is configured to control a focal length of illumination system 102 and observation system 104 to focus a test pattern image, such as first test pattern image 2100 with first compound slit 2102, at a plurality of locations relative to eye 10. Additionally, controller 300 of examination system 100 is configured to have first compound slit 2102 positioned at different offset values 2124 from leftside edge 2118 to move first compound slit 2102 transversely across eye 10.

In embodiments, controller 300 is further configured to determine a portion of the first test pattern image 2100 corresponding to a first anatomical region of eye 10. Referring to FIG. 72D, in one example, the first anatomical region of eye 10 is the limbus and the portion of first test pattern image 2100 is an area bounded by 2144 which corresponds to the determined location of limbus 802. The location of limbus 802 may be determined as explained in the present disclosure. In FIG. 72D, controller 300 has limited first region 2104 along to have upper extent 2142 at a boundary 2144 of limbus 802 and lower extent 2146 at boundary 2144 of limbus 802. Similarly, controller 300 has limited second region 2106 along to have upper extent 2148 at boundary 2144 of limbus 802 and lower extent 2150 at boundary 2144 of limbus 802. In embodiments, controller 300 may set each of upper extent 2142 and lower extent 2146 of first region 2104 and upper extent 2148 and lower extent 2150 of second region 2106 to be within boundary 2144 of limbus 802, such as up to 10 percent of the overall length of the respective first region 2104 and second region 2106. In embodiments, controller 300 may set each of upper extent 2142 and lower extent 2146 of first region 2104 and upper extent 2148 and lower extent 2150 of second region 2106 to extend beyond boundary 2144 of limbus 802, such as up to 10 percent of the overall length of the respective first region 2104 and second region 2106. In embodiments, first region 2104 and second region 2106 each have an intensity profile that is uniform. In embodiments, first region 2104 and second region 2106 each have an intensity profile that is non-uniform. In one example, a central position along a height of the respective first region 2104 and second region 2106 may have a first intensity value and a position closer to the respective upper extent 2142, upper extent 2148 and/or a position closer to the respective lower extent 2146, lower extent 2150 may have a second intensity value, the second intensity value being lower than the first intensity value. For example, a central portion of each of first region 2104 and second region 2106 may have a generally constant intensity value for a first height extent, such as 80 percent, a declining intensity value down to background intensity 2130 at a second height extent between the first height extent and each of upper extent 2142 and upper extent 2148, and a declining intensity value down to background intensity 2130 at a third height extent between the first height extent and each of lower extent 2146 and lower extent 2150. In embodiments, wherein controller 300 extends each of first region 2104 and second region 2106 outside of boundary 2144, an intensity value of a respective position of first region 2104 and second region 2106 outside of boundary 2144 may have a lower intensity than a respective position of first region 2104 and second region 2106 within boundary 2144.

Referring to FIG. 73A, a second test pattern image 2200 of projector 402 is shown. Second test pattern image 2200 includes an example of a second compound slit 2202 which is analogous to compound slit 2102 with a space between first region 2104 and second region 2106. Compound slit 2202 includes first region 2104 and second region 2106 of compound slit 2102 and a third region 2204 positioned between first region 2104 and second region 2106. Third region 2204 is has a width 2206 bounded by second vertical edge 2112 of first region 2104 and an edge 2117 of second region 2106.

In embodiments, the intensity value of third region 2204 is less than second intensity level 2134 of second region 2106. Referring to FIG. 73B, third region 2204 has an intensity value equal to background intensity 2130. In embodiments, width 2206 of third region 2204 is equal to first width 2120 of first region 2104. In embodiments, width 2206 of third region 2204 is less than first width 2120 of first region 2104. In embodiments, width 2206 of third region 2204 is greater than first width 2120 of first region 2104. In embodiments, width 2206 of third region 2204 is more than twice first width 2120 of first region 2104. In embodiments, width 2206 of third region 2204 is more than thrice first width 2120 of first region 2104. In embodiments, width 2206 of third region 2204 is more than five times first width 2120 of first region 2104. In embodiments, width 2206 of third region 2204 is more than ten times first width 2120 of first region 2104. In embodiments, width 2206 of third region 2204 is more than twenty-five times first width 2120 of first region 2104. In embodiments, width 2206 of third region 2204 is more than forty times first width 2120 of first region 2104. In embodiments, width 2206 of third region 2204 is up to fifty times first width 2120 of first region 2104. In embodiments, width 2206 of third region 2204 is between about 0.5 to about 4.0 times first width 2120 of first region 2104. In embodiments, width 2206 of third region 2204 is in the range of about 0.5 millimeters to about 3.0 millimeters. In embodiments, width 2206 of third region 2204 is in the range of about 0.04 millimeters to about 0.8 millimeters. An advantage, among others, of including third region 2204 is improved contrast in the images captured by examination system 100.

Compound slit 2202, like first compound slit 2102, may have either uniform or non-uniform intensity profiles along a height direction. Additionally, a height extent of second compound slit 2202, like first compound slit 2102, may be limited by controller 300 based on determined location of one or more anatomical features of eye 10, such as the limbus.

Referring to FIG. 74A, a third test pattern image 2300 of projector 402 is shown. Third test pattern image 2300 includes an example of a third compound slit 2302 which is analogous to compound slit 2102 with a fourth region bounding the opposite side of second region 2106 from first region 2104. Compound slit 2302 includes first region 2104 and second region 2106 of compound slit 2102 and a fourth region 2304 positioned such that first region 2104 is bounded by first region 2104 and fourth region 2304. Fourth region 2304 has a width 2306 bounded by first vertical edge 2114 of second region 2106 and an edge 2308.

In embodiments, the intensity value 2310 of fourth region 2304 is greater than second intensity level 2134 of second region 2106. Referring to FIG. 74B, fourth region 2304 has an intensity value 2310 equal to first intensity level 2132. In embodiments, width 2306 of fourth region 2304 is equal to first width 2120 of first region 2104. In embodiments, width 2306 of fourth region 2304 is less than first width 2120 of first region 2104. In embodiments, width 2306 of fourth region 2304 is greater than first width 2120 of first region 2104. In embodiments, width 2306 of fourth region 2304 is in the range of about 0.04 millimeters to about 0.8 millimeters. In embodiments, second width 2122 of second region 2106 is more than twice width 2306 of fourth region 2304. In embodiments, second width 2122 of second region 2106 is more than thrice width 2306 of fourth region 2304. In embodiments, second width 2122 of second region 2106 is more than five times width 2306 of fourth region 2304. In embodiments, second width 2122 of second region 2106 is more than ten times width 2306 of fourth region 2304. In embodiments, second width 2122 of second region 2106 is more than twenty-five times width 2306 of fourth region 2304. In embodiments, second width 2122 of second region 2106 is more than forty times width 2306 of fourth region 2304. In embodiments, second width 2122 of second region 2106 is up to fifty times width 2306 of fourth region 2304. In embodiments, second width 2122 of second region 2106 is in the range of about 0.5 millimeters to about 3.0 millimeters. An advantage, among others, of including fourth region 2304 is to reduce the time needed to complete an examination as multiple thin slits (first region 2104 and fourth region 2304) are projected on eye 10. Due to the increased light from first region 2104 and fourth region 2304, the intensity value 2134 of second region 2106 may be less than in the case of first compound slit 2102.

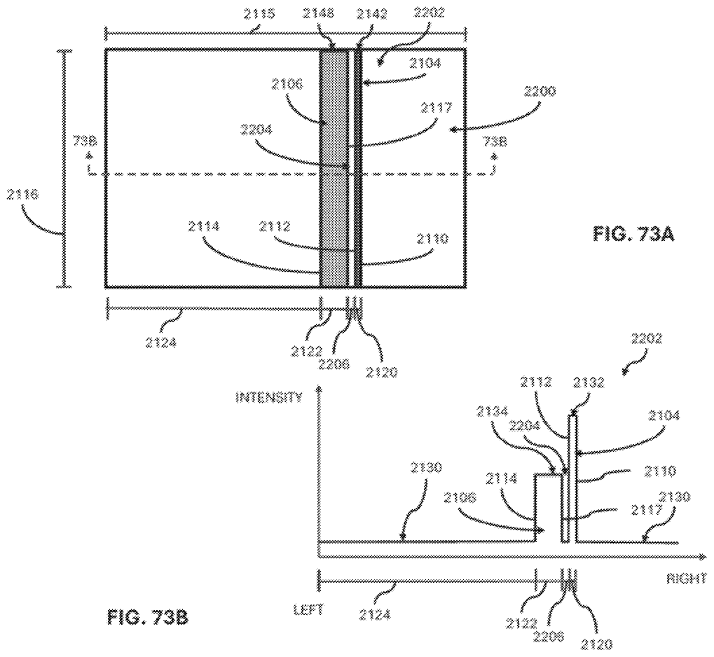

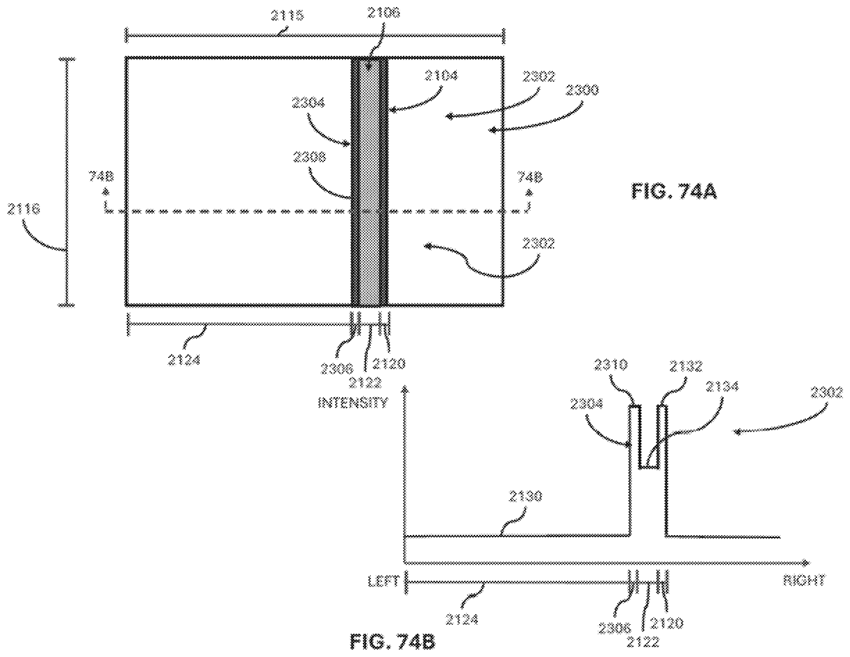

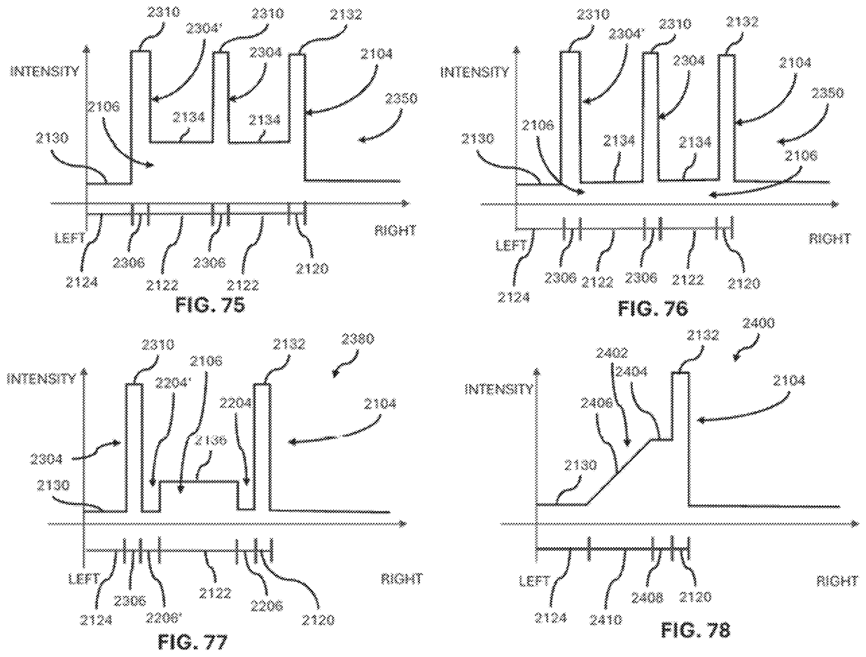

In embodiments, multiple instances of second region 2106 and fourth region 2304 are provided to further reduce the time needed to complete an examination. Referring to FIG. 75, an intensity profile for a fourth compound slit 2350 is shown having two instances of second region 2106 (2106 and 2106′) and two instances of fourth region 2304 (2304 and 2304′). Due to the increased light from first region 2104 and the two instances of fourth region 2304, the intensity value 2134 of second region 2106 may be less than in the case of first compound slit 2102 and the case of third compound slit 2302. In one example, shown in FIG. 76, the intensity value of the two instances of second region 2106 is reduced to equal background intensity 2130.

Compound slits 2302 and 2350, like first compound slit 2102, may have either uniform or non-uniform intensity profiles along a height direction. Additionally, a height extent of compound slits 2302 and 2350, like first compound slit 2102, may be limited by controller 300 based on determined location of one or more anatomical features of eye 10, such as the limbus. A non-uniform intensity profile might be useful to avoid oversaturation of portions of the image that can be caused by increased reflectivity of the perilimbal zone in conditions like arcus senilis.

Referring to FIG. 77, an intensity plot of a fifth compound slit 2380 is shown. Fifth compound slit 2380 is analogous to compound slit 2302 with third region 2204 between first region 2104 and second region 2106 and a second instance of third region 2204′ between second region 2106 and fourth region 2304.

Referring to FIG. 78, an intensity plot of a sixth compound slit 2400 is shown. Sixth compound slit 2400 is analogous to compound slit 2102 with a different second region 2402. Second region 2402 includes a non-uniform intensity profile along a width direction of 2124. Referring to FIG. 78, an exemplary intensity profile includes a uniform portion having a width 2408 and an intensity level 2404 which is less than intensity value 2132 and a linear ramp portion having a width of 2410 and an intensity value that transitions from background intensity 2130 to intensity level 2404. Other exemplary non-uniform profiles are contemplated. In embodiments, sixth compound slit 2400 includes a third region 2204 between first region 2104 and second region 2402.

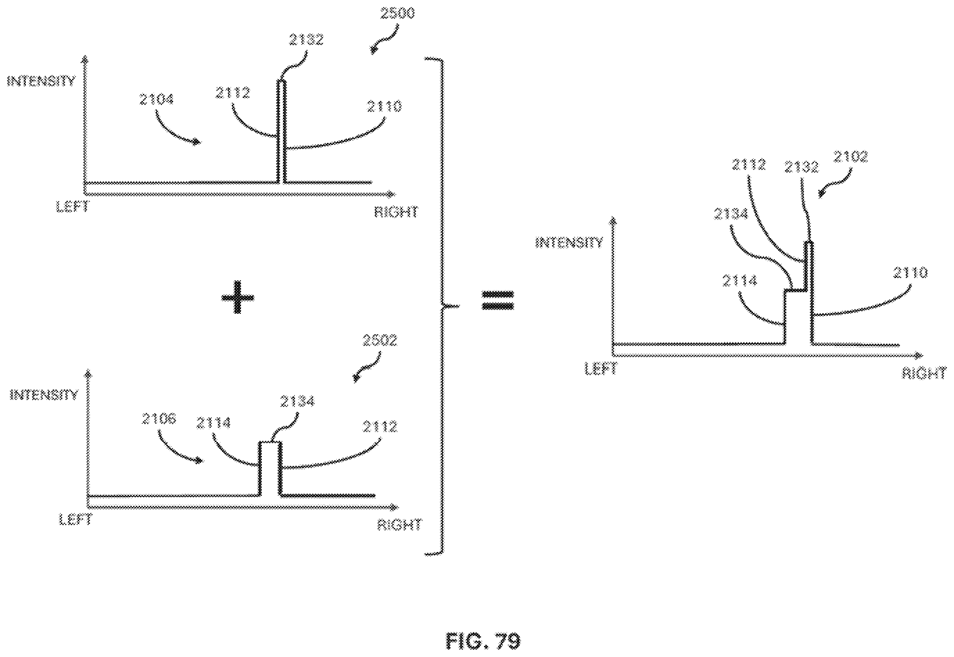

Referring to FIG. 79, in embodiments, controller 300 provides an image of compound slit 2102 by first illuminating eye 10 with a test pattern image having an intensity profile 2500 that includes a first portion of 2102, illustratively first region 2104. Controller 300 receives a first image of eye 10, such as of the corneal region, corresponding to eye 10 being illuminated with the test pattern image having the intensity profile 2500. Controller 300 illuminates eye 10 with a test pattern image having an intensity profile 2502 that includes a second portion of 2102, illustratively second region 2106. Controller 300 receives a second image of eye 10, such as of the corneal region, corresponding to eye 10 being illuminated with the test pattern image having the intensity profile 2502. Controller 300 combines the first image and the second image to produce an image of the eye illuminated by both the first portion of the compound slit 2102 and the second portion of the compound slit 2102 to emulate illuminating eye 10 with first test pattern image 2100 of FIG. 72A. In embodiments, the first and second images are registered and superimposed. In embodiments, the relative brightness of the first and second images can be adjusted during blending to increase the details of the eye 10 under examination. In embodiments, the first image acquired while illuminating with first region 2104 is extracted (image segmentation) and added to the second image acquired while illuminating with second region 2106. In embodiments the relative brightness of the extracted portion of the first image may be adjusted to optimize contrast. In embodiments, the extracted portion of the first image may undergo a non-linear intensity transformation such as gamma correction to enhance visibility of pathologies without image saturation. In embodiments, the other disclosed compound slits may be emulated in a similar way by combining two or more images.

In embodiments, the first portion of the compound slit shown in intensity profile 2500 corresponds to a first region with a first intensity and the second portion of the compound slit shown in intensity profile 2502 corresponds to a second region with a second intensity. The second intensity being less than the first intensity. An area of the second region being greater than an area of the first region. In examples, the first region of the compound slit abuts the second region of the compound slit. In examples, the first region of the compound slit is spaced apart from the second region of the compound slit. Each of the first region of the compound slit and the second region of the compound slit may be generally rectilinear in shape. Each of the first region of the compound slit and the second region of the compound slit may extend outside of the corneal region of the eye. Each of the first region of the compound slit and the second region of the compound slit may be confirmed generally to the corneal region of the eye.

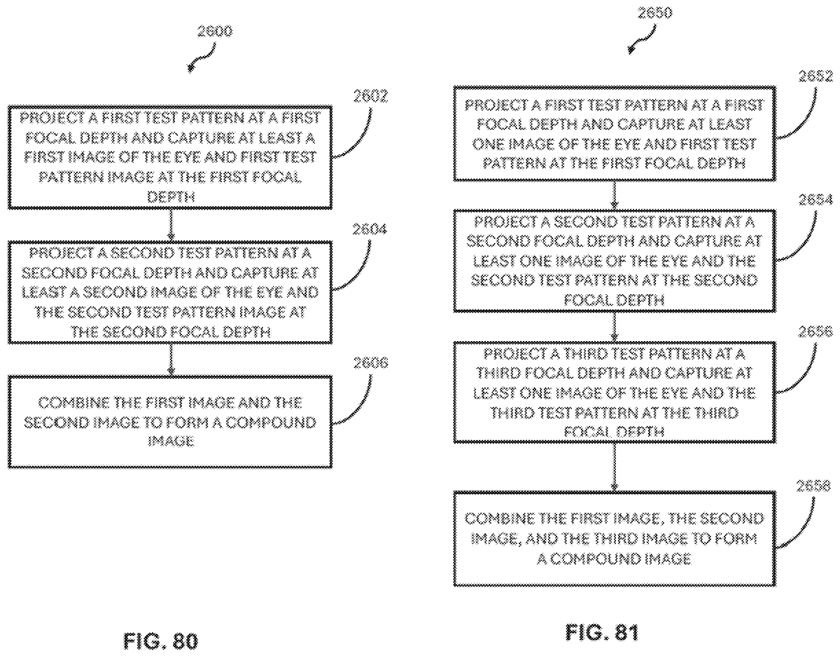

Referring to FIG. 80, exam logic 320 of controller 300 is configured to execute the processing sequence 2600. Controller 300 projects a first test pattern image with the illumination system 102 focused at a first focal depth and the observation system 104 focused at a first focal depth, as represented by block 2602. Controller 300 captures at least a first image with the observation system while the first test pattern image is being projected. Controller 300 projects a second test pattern image different from the first test pattern image with the illumination system 102 focused at a second focal depth and the observation system 104 focused at a second focal depth, as represented by block 2604. Controller 300 captures at least a second image with the observation system while the second test pattern image is being projected. Controller 300 combines the first image and the second image to form a compound image, as represented by block 2606. In embodiments, at least one of the first test pattern image and the second test pattern image includes a compound slit, such as compound slit 2102 or second compound slit 2202. In embodiments, the second test pattern image includes first compound slit 2102 and the first test pattern image includes only first region 2104 of first compound slit 2102 and omits second region 2106 of first compound slit 2102.

Referring to FIG. 81, exam logic 320 of controller 300 is configured to execute the processing sequence 2650. Controller 300 projects a first test pattern image with the illumination system 102 focused at a first focal depth and the observation system 104 focused at a first focal depth, as represented by block 2652. Controller 300 captures at least a first image with the observation system while the first test pattern image is being projected. Controller 300 projects a second test pattern image with the illumination system 102 focused at a second focal depth and the observation system 104 focused at a second focal depth, as represented by block 2654. Controller 300 captures at least a second image with the observation system while the second test pattern image is being projected. In embodiments, the second test pattern image is different than the first test pattern image. Controller 300 projects a third test pattern image with the illumination system 102 focused at a third focal depth and the observation system 104 focused at a third focal depth, as represented by block 2656. Controller 300 captures at least a third image with the observation system while the third test pattern image is being projected. Controller 300 combines the first image, the second image, and the third image to form a compound image, as represented by block 2658. In embodiments, the third test pattern image is the same as one of the first test pattern image and the second test pattern image. In embodiments, the third test pattern image is the same as the second test pattern image.

In embodiments, at least one of the first test pattern image and the second test pattern image includes a compound slit, such as compound slit 2102 or second compound slit 2202. In embodiments, the first test pattern image includes one of first compound slit 2102 and second compound slit 2202 and the second test pattern image includes only first region 2104 of first compound slit 2102 or second compound slit 2202 and omits second region 2106 of first compound slit 2102 or second compound slit 2202.

In embodiments, the first test pattern image includes one of first compound slit 2102 and second compound slit 2202 and both the second test pattern image and the third test pattern image includes only first region 2104 of first compound slit 2102 or second compound slit 2202 and omits second region 2106 of first compound slit 2102 or second compound slit 2202. In one example, the first focal depth of the illumination system corresponds generally to an anterior surface of a lens of the eye, the second focal depth of the illumination system corresponds generally to a cornea of the eye, and the third focal depth of the illumination system corresponds generally to a posterior surface of the lens of the eye.

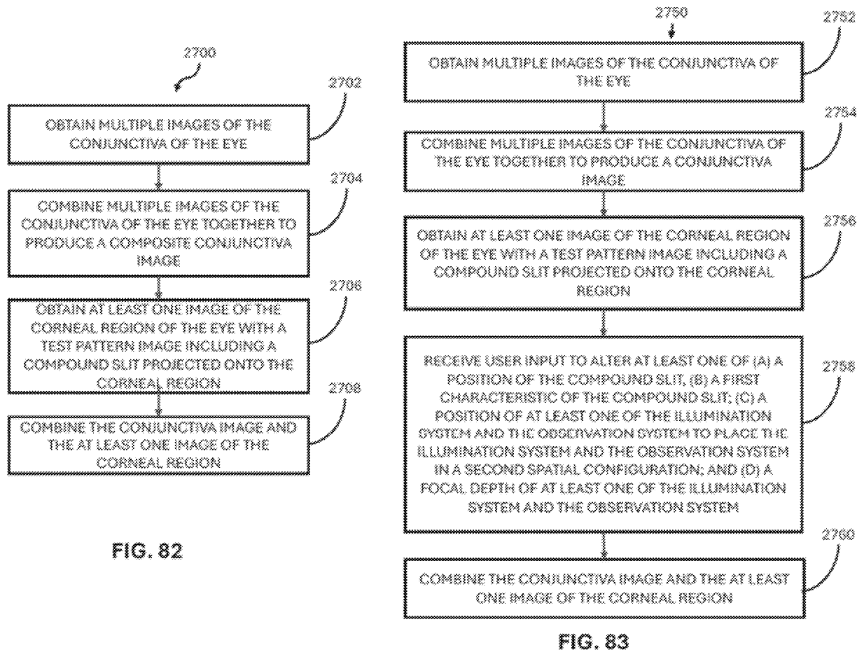

Referring to FIG. 82, exam logic 320 of controller 300 is configured to execute the processing sequence 2700. Controller 300 projects a first test pattern image with the illumination system 102. In embodiments, the first test pattern image has a constant intensity value and is used to image the conjunctiva of eye 10. Observation system 104 is focused by controller 300 on the conjunctiva of eye 10. Controller 300 obtains multiple images of the conjunctiva of eye 10, as represented by block 2702. In embodiments, controller 300 obtains at least four images of the conjunctiva of eye 10 including a leftward gaze of the eye, a rightward gaze of the eye, an upward gaze of the eye, and a downward gaze of the eye.

Controller 300 combines the multiple images of the conjunctiva of eye 10 together to produce a composite conjunctiva image, as represented by block 2704. In embodiments, controller 300 stitches together portions of the multiple images of the conjunctiva of eye 10 to produce the composite conjunctiva image wherein overlapping portions of the conjunctiva are aligned to produce an image of the conjunctiva that surrounds a corneal region of eye 10.

Controller 300 also obtain at least one image of the corneal region of eye 10 when the corneal region is illuminated by a test pattern image including a compound slit, such as first compound slit 2102 or second compound slit 2202, as represented by block 2706. In embodiments, the compound slit in the test pattern image is limited in height to the corneal region. Controller 300 then combines the composite conjunctiva image and the at least one image of the corneal region to produce a composite image of eye 10, as represented by block 2708. The composite image of eye 10 may be imported into a medical record for the patient.

In embodiments, controller 300 obtains multiple images of the corneal region and combines them with the composite conjunctiva image. For example, controller 300 make obtain images at different focal depths, such as a first image focused at the corneal surface, a second image focused at the anterior side of the lens, and a third image focused at a posterior side of the lens.

Referring to FIG. 83, exam logic 320 of controller 300 is configured to execute the processing sequence 2750. Controller 300 projects a first test pattern image with the illumination system 102. In embodiments, the first test pattern image has a constant intensity value and is used to image the conjunctiva of eye 10. Observation system 104 is focused by controller 300 on the conjunctiva of eye 10. Controller 300 obtains multiple images of the conjunctiva of eye 10, as represented by block 2752. In embodiments, controller 300 obtains at least four images of the conjunctiva of eye 10 including a leftward gaze of the eye, a rightward gaze of the eye, an upward gaze of the eye, and a downward gaze of the eye.

Controller 300 combines the multiple images of the conjunctiva of eye 10 together to produce a composite conjunctiva image, as represented by block 2754. In embodiments, controller 300 stitches together portions of the multiple images of the conjunctiva of eye 10 to produce the composite conjunctiva image wherein overlapping portions of the conjunctiva are aligned to produce an image of the conjunctiva that surrounds a corneal region of eye 10.

Controller 300 also obtain at least one image of the corneal region of eye 10 when the corneal region is illuminated by a test pattern image including a compound slit, such as first compound slit 2102 or second compound slit 2202, as represented by block 2756. In embodiments, the compound slit in the test pattern image is limited in height to the corneal region. In embodiments, controller 300 receives a user input, such as through input devices 360, to alter at least one of (a) a position of the compound slit, (b) a first characteristic of the compound slit; (c) a position of at least one of the illumination system and the observation system to place the illumination system and the observation system in a second spatial configuration; and (d) a focal depth of at least one of the illumination system and the observation system, as represented by block 2758. In examples, the displayed corneal image is a live image and the input is to examination system 100. In examples, the displayed corneal image is a stored image post examination and in response to the input controller 300 retrieves another image from a library of images obtained during examination based on the requested input and replaces the current corneal image. Systems and methods to classify, retrieve, and display images are disclosed in U.S. Pat. No. 11,911,109, the entire disclosure of which is expressly incorporated by reference herein.

Controller 300 then combines the composite conjunctiva image and the at least one image of the corneal region to produce a composite image of eye 10, as represented by block 2760. The composite image of eye 10 may be imported into a medical record for the patient.

In embodiments, controller 300 obtains multiple images of the corneal region and combines them with the composite conjunctiva image. For example, controller 300 make obtain images at different focal depths, such as a first image focused at the corneal surface, a second image focused at the anterior side of the lens, and a third image focused at a posterior side of the lens.

The following Examples provide non-limiting embodiments of the present disclosure.

Example 1. A method of examining an eye of a patient is provided. The method comprising the steps of: positioning an illumination system and an observation system of an examination system in a first spatial configuration relative to the eye; determining that a scan definition defining a plurality of images to be captured by at least one camera of the observation system may be executed with the illumination system and the observation system in the first spatial configuration, the plurality of images including a first image at a first focal plane of the eye and a second image at a second focal plane of the eye; and while maintaining the illumination system and the observation system in the first spatial configuration performing the following steps: (a) projecting a first pattern from the illumination system towards the eye, (b) receiving light reflected by the eye from the projecting step (a) with a first camera of the observation system to capture the first image of the plurality of images, the illumination system and the observation system both focused at the first focal plane relative to the eye during steps (a) and (b); (c) projecting a second pattern from the illumination system towards the eye, (d) receiving light reflected by the eye from the projecting step (c) with the first camera of the observation system to capture the second image of the plurality of images, the illumination system and the observation system both focused at the second focal plane relative to the eye during steps (c) and (d).

Example 2. The method of Example 1, further comprising tracking a position of the eye; determining a first movement of the eye prior to step (c); and adjusting at least one characteristic of the illumination system based on the determined movement of the eye.

Example 3. The method of Example 2, wherein the step of adjusting the at least one characteristic of the illumination system based on the determined first movement of the eye includes adjusting a focal plane of the illumination system.

Example 4. The method of any one of Examples 2 and 3, wherein the step of adjusting the at least one characteristic of the illumination system based on the determined first movement of the eye includes adjusting a feature of the second pattern.

Example 5. The method of Example 4, wherein the feature of the second pattern is a line of an image produced with a digital projector of the illumination system.

Example 6. The method of Example 5, wherein an offset of the line from an edge of the image is adjusted.

Example 7. The method of any of Examples 2-6, wherein the step of adjusting the at least one characteristic of the illumination system based on the determined first movement of the eye includes adjusting an adjustable optical assembly of the illumination system.

Example 8 The method of Example 7, wherein the step of adjusting the adjustable optical assembly of the illumination system includes the step of adjusting a focal length of a tunable lens.

Example 9 The method of Example 8, wherein the step of adjusting the adjustable optical assembly of the illumination system includes the step of adjusting a position of a movable lens.

Example 10. The method of any of Examples 2-6, further comprising the step of adjusting at least one characteristic of the observation system based on the determined first movement of the eye.

Example 11. The method of Example 10, wherein the step of adjusting the at least one characteristic of the observation system based on the determined movement of the eye includes adjusting a focal plane of the observation system.

Example 12. The method of any of Examples 10 and 11, wherein the step of adjusting the at least one characteristic of the observation system based on the determined first movement of the eye includes adjusting an adjustable optical assembly of the observation system.

Example 13. The method of Example 12, wherein the step of adjusting the adjustable optical assembly of the observation system includes the step of adjusting a focal length of a tunable lens.

Example 14. The method of Example 13, wherein the step of adjusting the adjustable optical assembly of the observation system includes the step of adjusting a position of a movable lens.

Example 15. The method of any of Examples 1-14, further comprising the steps of determining a second movement of the eye causes at least a portion of the scan definition to be outside of a scan envelope of the examination system with the illumination system and the observation system in the first spatial configuration; and adjusting a position of the illumination system and a position of the observation system of the examination system to a second spatial configuration relative to the eye whereat the portion of the scan definition is within the scan envelope of the examination system, the second spatial configuration being different than the first spatial configuration.

Example 16. The method of Example 15, wherein the step of adjusting the position of the illumination system and the observation system of the examination system to the second spatial configuration relative to the eye includes the step of adjusting a position of a movable base of the examination system supporting the illumination system and the observation system relative to a patient support of the examination system, the patient support being adapted to support the eye of the patient.

Example 17. The method of any one of Examples 11-16, wherein step of adjusting the focal plane of the observation system includes analyzing at least one image captured by a camera with an image sensor tilted relative to a focal plane of the observation system.

Example 18. The method of any one of Examples 11-16, wherein step of adjusting the focal plane of the observation system includes analyzing a plurality of images captured by a camera of the observation system, each image being at a respective focal depth and further comprising the step of analyzing a characteristic of each image to determine a position of the examination system relative to the eye.

Example 19. A medical device for imaging at least a portion of an eye of a patient is provided. The medical device comprising: an illumination system including a digital projector producing at least one test pattern image to illuminate the eye and a first adjustable optical assembly to project the at least one test pattern image towards the eye and an observation system including a second adjustable optical assembly positioned to receive imaging rays produced by reflection of light from the eye of the patient due to the projection of the at least one test pattern image towards the eye by the illumination system and a first camera configured receive the imaging rays from the second adjustable optical assembly. The illumination system and the observation system being positioned in a first spatial configuration relative to the eye. The medical device further comprising a controller operatively coupled to the illumination system and to the observation system, the controller configured to adjust the first adjustable optical assembly of the illumination system and the second adjustable optical system of the observation system to a plurality of focal depths relative to the eye while the illumination system and observation system are in the first spatial configuration. The controller further configured to (a) determine that a scan definition defining a plurality of images to be captured by at least one camera of the observation system may be executed with the illumination system and the observation system in the first spatial configuration, the plurality of images including a first image at a first focal plane and a second image at a second focal plane, the second focal plane being offset from the first focal plane; and while maintaining the illumination system and the observation system in the first spatial configuration the controller further configured to perform the following steps: (b) projecting a first pattern from the illumination system towards the eye with a first test pattern image of the at least one test pattern image produced by the digital projector, (c) receiving light reflected by the eye from the projecting step (b) with the first camera of the observation system to capture the first image of the plurality of images, the illumination system and the observation system both focused at the first focal plane relative to the eye during steps (b) and (c); (d) projecting a second pattern from the illumination system towards the eye with a second test pattern image of the at least one test pattern image produced by the digital projector, and (e) receiving light reflected by the eye from the projecting step (d) with the first camera of the observation system to capture the second image of the plurality of images, the illumination system and the observation system both focused at the second focal plane relative to the eye during steps (d) and (e).

Example 20. The medical device of Example 19, wherein the controller is further configured to perform the following steps: tracking a position of the eye; determining a first movement of the eye prior to step (d); and adjusting at least one characteristic of the illumination system based on the determined movement of the eye.

Example 21. The medical device of Example 20, wherein the adjusting of the at least one characteristic of the illumination system based on the determined first movement of the eye includes adjusting a focal plane of the illumination system.

Example 22. The medical device of any one of Examples 20 and 21, wherein adjusting the at least one characteristic of the illumination system based on the determined first movement of the eye includes adjusting a feature of the second pattern of the second test pattern image of the at least one test pattern image produced by the digital projector.

Example 23. The medical device of Example 22, wherein the feature of the second pattern is a line of the second test pattern image produced with the digital projector of the illumination system.

Example 24. The medical device of Example 23, wherein an offset of the line from an edge of the image is adjusted.

Example 25. The medical device of any of Examples 19-24, wherein the controller is further configured to perform the following step of adjusting at least one characteristic of the observation system based on the determined first movement of the eye.

Example 26. The medical device of Example 25, wherein the step of adjusting the at least one characteristic of the observation system based on the determined movement of the eye includes adjusting a focal plane of the observation system.

Example 27. The medical device of any of Examples 19-26, wherein the controller is further configured to perform the following steps determining a second movement of the eye causes at least a portion of the scan definition to be outside of a scan envelope of the examination system with the illumination system and the observation system in the first spatial configuration; and adjusting a position of the illumination system and a position of the observation system of the examination system to a second spatial configuration relative to the eye whereat the portion of the scan definition is within the scan envelope of the examination system, the second spatial configuration being different than the first spatial configuration.

Example 28. The medical device of Example 27, further comprising: a movable base supporting the illumination system and the observation system; and a patient support adapted to support the eye of the patient.

Example 29. The medical device of Example 28, wherein the controller is further configured to perform the step of adjusting a position of the movable base relative to the patient support to position the illumination system and the observation system in the second spatial configuration relative to the eye.

Example 30. A medical device for determining a separation from an eye. The medical device comprising: an optical system positioned to receive reflected light from the eye, the optical system having an optical axis; an image sensor positioned to detect an image formed by the optical system, the image sensor tilted relative to the optical axis of the optical system such that a first portion of the image sensor on a first side of a plane passing through the optical axis is closer to a rear face of the optical system than a second portion of the image sensor on a second side of the plane passing through the optical axis, the second side of the plane being opposite the first side of the plane; and a controller configured to receive image data of the detected image from the image sensor and to determine the separation from the eye based on analyzing multiple portions of image data including at least one of a first portion of the image data from the first portion of the image sensor and a second portion of the image data from the second portion of the image sensor.

Example 31. The medical device of Example 30, wherein the controller further analyzes at least a third portion of the image data, the third portion of image data being closer to the plane than the at least one of the first portion of the image data from the first portion of the image sensor and the second portion of the image data from the second portion of the image sensor.

Example 32. The medical device of any one of Examples 30 and 31, wherein the controller analyzes both the first portion of the image data from the first portion of the image sensor and the second portion of the image data from the second portion of the image sensor.

Example 33. The medical device of any one of Examples 30-32, wherein the controller analyzes a frequency component of the image data to determine the separation from the eye.

Example 34. The medical device of Example 33, wherein the frequency component is a frequency domain component.

Example 35. The medical device of any one of Examples 30-33, wherein the controller is configured to: (a) convert the image data to a grayscale image data; (b) determine a frequency domain of the grayscale image data; (c) apply a first high pass filter to the frequency domain of the grayscale image to produce a high frequency domain image; (d) apply a second high pass filter to the frequency domain of the grayscale image to produce a mid-to-high frequency domain image; (e) determine a first spatial image of the high frequency domain image; (f) determine a second spatial image of the mid-to-high frequency domain image; (g) for each pixel of the image data determine a ratio of the pixel value in the first spatial image to the pixel value in the second spatial image to produce a focus map; (h) determine a centroid of the focus map; and (i) based on a position of the centroid determine the separation from the eye.

Example 36. The medical device of 35, wherein the separation from the eye is determined by determining a pixel separation of the centroid from the plane.

Example 37. The medical device of Example 36, wherein the pixel separation is converted to a distance.

Example 38. A medical device for determining a separation from an eye. The medical device comprising: an optical system positioned to receive reflected light from the eye, the optical system having an optical axis and at least one adjustable lens; an image sensor positioned to detect a plurality of images formed by the optical system, each image of the plurality of images at a different focal length of the optical system; and a controller configured to receive image data of the detected plurality of images from the image sensor and to determine the separation from the eye based on analyzing the plurality of images.

Example 39. The medical device of Example 38, wherein the controller analyzes a frequency component of the image data of each of the plurality of images to determine the separation from the eye.

Example 40. The medical device of Example 39, wherein the frequency component is a frequency domain component.

Example 41. The medical device of Example 38, wherein the controller is configured to determine a sharpness characteristic for each image of the plurality of images.

Example 42. The medical device of Example 41, wherein controller determines the sharpness characteristic for each image of the plurality of images by: (a) converting the image data to a grayscale image data; (b) determining a frequency domain of the grayscale image data; (c) applying a first high pass filter to the frequency domain of the grayscale image to produce a high frequency domain image; (d) applying a second high pass filter to the frequency domain of the grayscale image to produce a mid-to-high frequency domain image; (e) determining a first spatial image of the high frequency domain image; (f) determining a second spatial image of the mid-to-high frequency domain image; (g) determine a characteristic based on the first spatial image and the second spatial image; and (h) based on the characteristic based on the first spatial image and the second spatial image determining the separation from the eye.

Example 43. The medical device of Example 42, wherein the characteristic based on the first spatial image and the second spatial image is a ratio of a summation of each pixel value in the first spatial image to a summation of each pixel value in the second spatial image.

Example 44. The medical device of any one of Examples 42 and 43, wherein a first image of the plurality of images is selected based on having an extreme value of the characteristic based on the first spatial image and the second spatial image and the separation from the eye is determined based on a characteristic of the at least one adjustable lens corresponding to the first image.

Example 45. The medical device of Example 41, wherein controller determines the sharpness characteristic for each image of the plurality of images by: (a) converting the image data to a grayscale image data; (b) normalize the image data; (c) determining a characteristic of the normalized image data; and (d) based on the characteristic of the normalized image data determining the separation from the eye.

Example 46. The medical device of Example 45, wherein the step of determining the characteristic of the normalized image data includes the steps of: determining a convolution of the image data with an edge detection kernel; and summing the pixel values resulting from the step of determining the convolution of the image data with the edge detection kernel.

Example 47. The medical device of Example 45, wherein the step of determining the characteristic of the normalized image data includes the step of determining a variance characteristic of the normalized image data.

Example 48. The medical device of any one of Examples 45-48, wherein a first image of the plurality of images is selected based on having an extreme value of the characteristic of the normalized image data and the separation from the eye is determined based on a characteristic of the at least one adjustable lens corresponding to the first image.

Example 49. The medical device of Example 48, wherein the extreme value of the characteristic of the normalized image data is a maximum value of the characteristic of the normalized image data relative to a remainder of the plurality of images.

Example 50. The medical device of any one of Examples 38-49, wherein the controller is further configured to determine based on the plurality of images a focal depth range of the medical device.

Example 51. The medical device of Example 50, wherein the focal depth range is determined by determining a characteristic of each image of the plurality of images and based on the determined characteristic of each image determining the separation from the eye.

Example 52. The medical device of Example 51, wherein the controller is further configured to determine a plurality of locations for illumination of the eye by the medical device, the plurality of locations being within the focal depth range.

Example 53. The medical device of any one of Examples 51 and 52, wherein the controller is further configured to determine a plurality of locations for imaging of the eye by the medical device, the plurality of locations being within the focal depth range.

Example 54. The medical device of any one of Examples 38-53, wherein the at least one adjustable lens of the optical system includes at least one tunable lens.

Example 55. The medical device of any one of Examples 38-54, wherein the at least one adjustable lens of the optical system includes at least one movable lens.

Example 56. The medical device of any one of Examples 38-55, further comprising: an illumination system including a digital projector producing at least one test pattern image to illuminate the eye and a first adjustable optical assembly to project the at least one test pattern image towards the eye; and an observation system including the optical system which is positioned to receive imaging rays produced by reflection of light from the eye of the patient due to the projection of the at least one test pattern image towards the eye by the illumination system and a first camera configured receive the imaging rays from the second adjustable optical assembly, the illumination system and the observation system being positioned in a first spatial configuration relative to the eye.

Example 57. The medical device of Example 56, wherein the at least one test pattern includes a vertical slit and in a first image of the at least one test pattern image the vertical slit is in a first transverse position and in a second image of the at least one test pattern image the vertical slit is in a second transverse position, the second traverse position in second image being spaced apart from the first transverse position in the first image.

Example 58. The medical device of Example 57, wherein the controller focuses the first adjustable optical assembly of the illumination system at a first focal depth of a plurality of focal depths when the first image of the at least one test pattern image is produced by the digital projector to focus the vertical slit in the first transverse position on a cornea of the eye at a first location offset from an apex of the cornea of the eye and focuses the first adjustable optical assembly of the illumination system at a second focal depth of the plurality of focal depths when the second image of the at least one test pattern image is produced by the digital projector to focus the vertical slit in the second transverse position on the cornea at a second location offset from the apex of the cornea, the second location on the cornea being offset from the first location on the cornea.

Example 59. The medical device of Example 58, wherein the controller further focuses the optical system of the observation system at the first focal depth of the plurality of focal depths when the first image of the at least one test pattern image is produced by the digital projector to focus the vertical slit in the first transverse position on the cornea at the first location offset from the apex of the cornea and focuses the optical system of the observation system at the second focal depth of the plurality of focal depths when the second image of the at least one image is produced by the digital projector to focus the vertical slit in the second transverse position on the cornea at the second location offset from the apex of the cornea.

Example 60. The medical device of Example 58, wherein the controller is configured to adjust the first adjustable optical assembly of the illumination system and the optical system of the observation system to a plurality of focal depths relative to an apex of a cornea of the eye while the illumination system and observation system are in the first spatial configuration, wherein a first subset of the plurality of focal depths are part of a first contour and a second subset of the plurality of focal depths are part of a second contour, the second contour being offset relative to the first contour, and wherein the digital projector produces the first test pattern image having the vertical slit in the first transverse position for a first focal depth of the first subset of the plurality of focal depths of the first contour and a first focal depth of the second subset of the plurality of focal depths of the second contour and the digital projector produces the second test pattern image having the vertical slit in the second transverse position for a second focal depth of the first subset of the plurality of focal depths and a second focal depth of second subset of the plurality of focal depths.

Example 61. The medical device of Example 60, wherein the first contour has a first shape and the second contour has a second shape, the second shape being different than the first shape.

Example 62. The medical device of Example 61, wherein the second shape is flatter than the first shape.

Example 63. The medical device of any one of Examples 56-62, further comprising a movable base, the illumination system being supported by the movable base and the observation system being supported by the movable base, the illumination system being movable relative to the movable base, the illumination system is rotatable relative to the observation system about a first rotation axis.

Example 64. The medical device of Example 63, wherein the controller is further configured to determine a movement of the eye during an examination and to alter a position of the movable base based on the movement of the eye.

Example 65. The medical device of Example 63, further comprising a patient support adapted to position the eye of the patient, wherein the movable base is moveable relative to the patient support.

Example 66. A medical device for imaging at least a portion of an eye of a patient is provided. The medical device comprising: an illumination system including a digital projector producing at least one test pattern image to illuminate the eye and a first adjustable optical assembly to project the at least one test pattern image towards the eye; an observation system including a second adjustable optical assembly positioned to receive imaging rays produced by reflection of light from the eye of the patient due to the projection of the at least one test pattern image towards the eye by the illumination system and a first camera configured receive the imaging rays from the second adjustable optical assembly; a movable base supporting the illumination system and the observation system; a patient support adapted to position the eye of the patient, wherein the movable base is moveable relative to the patient support; and a controller operatively coupled to the illumination system, the observation system, and the movable base, the controller configured to adjust the first adjustable optical assembly of the illumination system and the second adjustable optical system of the observation system to a plurality of focal depths relative to the eye with the movable base in a first position relative to the patient support and the controller further configured to adjust the second adjustable optical system of the observation system to a second plurality of focal depths with the movable base in a second position relative to the patient support, the second position being different than the first position, the second position being further from the patient support than the first position, the second plurality of focal depths each providing a respective overall image of the eye to be captured by the first camera.

Example 67. A medical device for imaging at least a portion of an eye of a patient is provided. The medical device comprising an illumination system including a digital projector producing at least one test pattern image to illuminate the eye and a first optical assembly to project a first test pattern image of the at least one test pattern image. The first test pattern image having an overall width and an overall height. The first test pattern image including a first region having a first intensity, a second region having a second intensity greater than the first intensity, and a third region having a third intensity greater than the second intensity. The medical device further comprising an observation system including a second optical assembly positioned to receive imaging rays produced by reflection of light from the eye of the patient due to the projection of the first test pattern image towards the eye by the illumination system and a first camera configured receive the imaging rays from the second optical assembly and a controller operatively coupled to the illumination system and to the observation system. The controller configured to adjust a focal depth of the illumination system and a focal depth of the observation system.

Example 68. The medical device of Example 67, wherein the first intensity is zero.

Example 69. The medical device of Example 67, wherein the first intensity is greater than zero.

Example 70. The medical device of any of Examples 67-69, wherein the second intensity of the second region is less than 75 percent of the third intensity of the third region.

Example 71. The medical device of any of Examples 67-69, wherein the second intensity of the second region is less than 50 percent of the third intensity of the third region.

Example 72. The medical device of any of Examples 67-69, wherein the second intensity of the second region is less than 30 percent of the third intensity of the third region.

Example 73. The medical device of any of Examples 67-72, wherein the first test pattern image includes a fourth region having a fourth intensity value, the fourth region positioned between the second region and the third region, the fourth intensity value being less than the second intensity value.

Example 74. The medical device of Example 73, wherein the fourth intensity value is generally equal to the first intensity value.

Example 75. The medical device of any of Examples 67-74, wherein the first test pattern image includes a fifth region having a fifth intensity value, the second region positioned between the third region and the fifth region, the fifth intensity value being greater than the second intensity value.

Example 76. The medical device of Example 75, wherein the fifth intensity value is generally equal to the third intensity value.

Example 77. The medical device of any of Examples 75 and 76, wherein the first test pattern image includes a sixth region having a sixth intensity value, the sixth region positioned between the second region and the fifth region, the sixth intensity value being less than the second intensity value.

Example 78. The medical device of Example 77, wherein the sixth intensity value is generally equal to the first intensity value.

Example 79. The medical device of any of Examples 67-74, wherein the first test pattern image includes a seventh region having a seventh width and positioned between the first region and the second region, the seventh region having an intensity profile along the seventh width which transitions from the first intensity value of the first region to the second intensity value of the second region.

Example 80. The medical device of Example 79, wherein the intensity profile of the seventh region is a linear profile.

Example 81. The medical device of any one of Examples 67-80, wherein the first region has a first height, the second region has a second height, and the third region has a third height, the second height of the second region being generally equal to the overall height of the first test pattern image and the third height of the third region being generally equal to the overall height of the first test pattern image.

Example 82. The medical device of Example 81, wherein at least one of (a) the second intensity is generally constant along the second height of the second region and (b) the third intensity is generally constant along the third height of the third region.

Example 83. The medical device of Example 81, wherein at least one of (a) the second intensity is part of a second intensity profile of the second region that has a first intensity value at a first height position along the second height and a second intensity value at a second height position along the second height spaced apart from the first height position, the second intensity value being less than the first intensity value and (b) the third intensity is part of a third intensity profile of the third region that has a third intensity value at a third height position along the third height and a fourth intensity value at a fourth height position along the third height spaced apart from the third height position, the fourth intensity value being less than the third intensity value.

Example 84. The medical device of Example 83, wherein the controller is further configured to determine a portion of the first test pattern image corresponding to a first anatomical region of the eye.

Example 85. The medical device of Example 84, wherein at least one of (a) both the first height position along the second height and the second height position along the second height are positioned within or on a boundary of the portion of the first test pattern image corresponding to the first anatomical region of the eye and (b) both the third height position along the third height and the fourth height position along the third height are positioned within or on a boundary of the portion of the first test pattern image corresponding to the first anatomical region of the eye.

Example 86. The medical device of Example 84, wherein at least one of (a) the first height position along the second height is positioned within or on a boundary of the portion of the first test pattern image corresponding to the first anatomical region of the eye and the second height position along the second height is positioned outside of the portion of the first test pattern image corresponding to the first anatomical region of the eye and (b) the third height position along the third height is positioned within or on a boundary of the portion of the first test pattern image corresponding to the first anatomical region of the eye and the fourth height is position outside of the portion of the first test pattern image corresponding to the first anatomical region of the eye.

Example 87. The medical device of any one of Examples 84-86, wherein the portion of the first test pattern image corresponds to a region bounded by the limbus of the eye.

Example 88. The medical device of any one of Examples 67-80, wherein at least the second region of the first test pattern image and the third region of the first test pattern image form a compound slit and the controller is further configured to determine a portion of the first test pattern image corresponding to a region bounded by the limbus of the eye and to limit a height of the compound slit to the portion of the first test pattern image corresponding to the region bounded by the limbus.

Example 89. The medical device of Example 88, wherein at least one of the fourth region, the fifth region, the sixth region, and the seventh region are part of the compound slit.

Example 90. The medical device of any one of Examples 88 and 89, wherein the compound slit has a generally constant width along a height direction.

Example 91. The medical device of any one of Examples 67-80, wherein at least the second region of the first test pattern image and the third region of the first test pattern image form a compound slit and the controller is further configured to determine a portion of the first test pattern image corresponding to a region bounded by the limbus of the eye and to conform the compound slit to generally coincide with a portion of a boundary of the region bounded by the limbus such that the compound slit is generally arcuate along the height direction.

Example 92. The medical device of any one of Examples 67-90, wherein the controller (a) projects the first test pattern image with the illumination system focused at a first focal depth and the observation system focused at a first focal depth, (b) captures at least a first image with the observation system while the first test pattern image is being projected, (c) projects a second test pattern image different from the first test pattern image with the illumination system focused at a second focal depth and the observation system focused at a second focal depth, and (d) captures at least a second image with the observation system while the second test pattern image is being projected.

Example 93. The medical device of Example 92, wherein the second test pattern image includes the third region of the first test pattern image and omits the second region of the first test pattern image.

Example 94. The medical device of any one of Examples 92 and 93, wherein the controller combines the at least first image and the at least second image to form a compound image.

Example 95. The medical device of any one of Examples 67-90, wherein the controller (a) projects the first test pattern image with the illumination system focused at a first focal depth and the observation system focused at a first focal depth, (b) captures at least a first image with the observation system while the first test pattern image is being projected, (c) projects a second test pattern image different from the first test pattern image with the illumination system focused at a second focal depth and the observation system focused at a second focal depth, (d) captures at least a second image with the observation system while the second test pattern image is being projected, (e) projects a third test pattern image different from the first test pattern image with the illumination system focused at a third focal depth and the observation system focused at a third focal depth, and (d) captures at least a third image with the observation system while the second test pattern image is being projected.

Example 96. The medical device of Example 95, wherein the third test pattern image is the same as the second test pattern image.

Example 97. The medical device of any one of Examples 95 and 96, wherein the second test pattern image includes the third region of the first test pattern image and omits the second region of the first test pattern image.

Example 98. The medical device of any one of Examples 95-97, wherein the controller combines the at least first image, the at least second image, and the at least third image to form a compound image.

Example 99. The medical device of any one of Examples 95-98, wherein the first focal depth of the illumination system corresponds generally to an anterior surface of a lens of the eye, the second focal depth of the illumination system corresponds generally to a cornea of the eye, and the third focal depth of the illumination system corresponds generally to a posterior surface of the lens of the eye.

Example 100. The medical device of any one of Examples 67-80, wherein the first region has a first height, the second region has a second height, and the third region has a third height, the second height of the second region being generally equal to the overall height of the first test pattern image and the third height of the third region being generally equal to the overall height of the first test pattern image.

Example 101. The medical device of any one of Examples 67-100, wherein the second region has a second width and the third region has a third width, the second width being about 0.5 millimeters to about 3.0 millimeters.

Example 102. The medical device of any one of Examples 67-101, wherein the third region has a third width and a third height, the third width being about 0.04 millimeters to about 0.8 millimeters.

Example 103. The medical device of any one of Examples 67-101, wherein the second region has a second width and the third region has a third width, the second width of the second region is greater than the third width of the third region.

Example 104. The medical device of Example 103, wherein the second width of the second region is at least twice the third width of the third region.

Example 105. The medical device of Example 103, wherein the second width of the second region is at least thrice the third width of the third region.

Example 106. The medical device of Example 103, wherein the second width of the second region is up to fifty times the third width of the third region.

Example 107. The medical device of any one of Examples 67-106, wherein the first optical assembly of the illumination system is a first adjustable optical assembly and the second optical assembly of the observation system is a second adjustable optical assembly.

Example 108. The medical device of Example 107, wherein the illumination system and the observation system being positioned in a first spatial configuration relative to the eye; and the controller is configured to adjust the first adjustable optical assembly of the illumination system and the second adjustable optical system of the observation system to a plurality of focal depths relative to the eye while the illumination system and observation system are in the first spatial configuration.

Example 109. The medical device of any one of Examples 67-108, wherein for a first focal depth the controller is configured to focus the third region of the test pattern image at the first focal depth.

Example 110. A medical device for imaging at least a portion of an eye of a patient is provided. The medical device comprising an illumination system including a digital projector producing at least one test pattern image to illuminate the eye and a first optical assembly to the at least one test pattern image towards the eye; an observation system including a second optical assembly positioned to receive imaging rays produced by reflection of light from the eye of the patient due to the projection of the first test pattern image towards the eye by the illumination system and a first camera configured receive the imaging rays from the second optical assembly; and a controller operatively coupled to the illumination system and to the observation system. The controller configured to receive at least one image of the corneal region of the eye from the observation system. The at least one image of the corneal region of the eye corresponding to the eye being illuminated with a compound slit produced by the digital projector of the illumination system. The compound slit having a first region with a first intensity and a second region with a second intensity. The second intensity being less than the first intensity. An area of the second region being greater than an area of the first region.

Example 111. The medical device of Example 110, wherein the controller is further configured to receive multiple images of the conjunctiva of the eye from the observation system, each image corresponding to a respective gaze of the eye.

Example 112. The medical device of Example 111, wherein the controller is further configured to combine the multiple images of the conjunctiva of the eye and the at least one image of the corneal region of the eye.

Example 113. The medical device of Example 112, wherein the multiple images of the conjunctiva of the eye are combined to form an image of the conjunctiva that surrounds the corneal region.

Example 114. The medical device of any one of Examples 110-113, wherein the controller receives an input to alter at least one of (a) a position of the compound slit, (b) a first characteristic of the compound slit; (c) a position of at least one of the illumination system and the observation system to place the illumination system and the observation system in a second spatial configuration; and (d) a focal depth of at least one of the illumination system and the observation system.

Example 115. The medical device of any one of Examples 110-114, wherein the first region of the compound slit abuts the second region of the compound slit.

Example 116. The medical device of any one of Examples 110-114, wherein the first region of the compound slit is spaced apart from the second region of the compound slit.

Example 117. The medical device of any one of Examples 110-116, wherein each of the first region of the compound slit and the second region of the compound slit are generally rectilinear in shape.

Example 118. The medical device of any one of Examples 110-117, wherein each of the first region of the compound slit and the second region of the compound slit extend outside of the corneal region of the eye.

Example 119. The medical device of any one of Examples 110-117, wherein each of the first region of the compound slit and the second region of the compound slit are confined generally to the corneal region of the eye.

Example 120. The medical device of any one of Examples 110-119, wherein for a first focal depth the controller is configured to focus the first region of the test pattern image at the first focal depth.

Example 121. A medical device for imaging at least a portion of an eye of a patient is provided. The medical device comprising an illumination system including a digital projector producing at least one test pattern image to illuminate the eye and a first optical assembly to the at least one test pattern image towards the eye; an observation system including a second optical assembly positioned to receive imaging rays produced by reflection of light from the eye of the patient due to the projection of the first test pattern image towards the eye by the illumination system and a first camera configured receive the imaging rays from the second optical assembly; and a controller operatively coupled to the illumination system and to the observation system. The controller configured to receive a first image of the corneal region of the eye corresponding to the eye being illuminated with a first portion of a compound slit produced by the digital projector of the illumination system, receive a second image of the corneal region of the eye corresponding to the eye being illuminated with a second portion of a compound slit produced by the digital projector of the illumination system, and combine the first image and the second image to produce an image of the eye illuminated by both the first portion of the compound slit and the second portion of the compound slit.

Example 122. The medical device of Example 121, wherein the first portion of the compound slit corresponds to a first region with a first intensity and the second portion of the compound slit corresponds to a second region with a second intensity, the second intensity being less than the first intensity, and an area of the second region being greater than an area of the first region.

Example 123. The medical device of Example 122, wherein the first region of the compound slit abuts the second region of the compound slit.

Example 124. The medical device of Example 122, wherein the first region of the compound slit is spaced apart from the second region of the compound slit.

Example 125. The medical device of any one of Examples 121-124, wherein each of the first region of the compound slit and the second region of the compound slit are generally rectilinear in shape.

Example 126. The medical device of any one of Examples 121-125, wherein each of the first region of the compound slit and the second region of the compound slit extend outside of the corneal region of the eye.

Example 127. The medical device of any one of Examples 121-125, wherein each of the first region of the compound slit and the second region of the compound slit are confined generally to the corneal region of the eye.

Example 128. The medical device of Example 66, wherein the first plurality of focal depths each providing a respective image of a portion of the eye to be captured by the first camera.

Example 129. The medical device of any one of Examples 66 and 128, wherein each of the respective overall images of the eye include a complete extent of the eye.

Example 130. A medical device for imaging at least a portion of an eye of a patient is provided. The medical device comprising an illumination system including a digital projector producing at least one test pattern image to illuminate the eye and a first adjustable optical assembly to project the at least one test pattern image towards the eye and a secondary illumination system including an illumination source and optics to project an output of the illumination source towards the eye. The medical device further comprising an observation system including a second adjustable optical assembly positioned to receive imaging rays produced by reflection of light from the eye of the patient due to the projection of the at least one test pattern image towards the eye by the illumination system when the illumination system, the secondary illumination system, and the observation system are positioned in a first spatial configuration relative to the eye and to receive imaging rays produced by reflection of light from the eye of the patient due to the projection of the output of the illumination source of the secondary illumination system when the illumination system, the secondary illumination system, and the observation system are positioned in a second spatial configuration relative to the eye. The observation system further including a first camera configured to receive the imaging rays from the second adjustable optical assembly. The medical device further comprising a controller operatively coupled to the illumination system, the secondary illumination system, and to the observation system. The controller configured to adjust the first adjustable optical assembly of the illumination system and the second adjustable optical system of the observation system to a plurality of focal depths relative to the eye while the illumination system, the secondary illumination system, and observation system are in the first spatial configuration.

Example 131. The medical device of Example 130, wherein the illumination source of the secondary illumination system is one of a laser diode, a fiber coupled diode laser, and a light emitting diode.

Example 132. The medical device of Example 130, wherein the illumination source of the secondary illumination system emits a white illumination, a red illumination, or a near infrared illumination.

Example 133. The medical device of any one of Examples 130-132, wherein a first optical axis directed at the eye of the illumination system and a second optical axis directed at the eye of the secondary illumination system are fixed relative to each other.

Example 134. The medical device of Example 133, wherein the illumination system and the secondary illumination system rotate together relative to the eye. In a first rotational position of illumination system and the secondary illumination system the illumination system is aligned with the eye. In a second rotational position of the illumination system and the secondary illumination system the secondary illumination system is aligned with eye.

Example 135. The medical device of Example 134, wherein the first optical axis directed at the eye of the illumination system and the second optical axis directed at the eye of the secondary illumination system are angled relative to each other. Optionally the first optical axis directed at the eye of the illumination system is angled at about 10 degrees to the second optical axis directed at the eye of the secondary illumination system.

Example 136. The medical device of Example 135, wherein in the second rotational position of the illumination system and the secondary illumination system the illumination system is outside of a field of view of the first camera and the optics of the secondary illumination system are within the field of view of the first camera.

Example 137. The medical device of any one of Examples 19-29 and 56-129, further comprising a secondary illumination system including an illumination source and optics to project an output of the illumination source towards the eye.

Example 138. The medical device of Example 137, wherein the illumination source of the secondary illumination system is one of a laser diode, a fiber coupled diode laser, and a light emitting diode.

Example 139. The medical device of Example 137, wherein the illumination source of the secondary illumination system emits a white illumination, a red illumination, or a near infrared illumination.

Example 140. The medical device of any one of Examples 137-139, wherein a first optical axis directed at the eye of the illumination system and a second optical axis directed at the eye of the secondary illumination system are fixed relative to each other.

Example 141. The medical device of Example 140, wherein the illumination system and the secondary illumination system rotate together relative to the eye. In a first rotational position of illumination system and the secondary illumination system the illumination system is aligned with the eye. In a second rotational position of the illumination system and the secondary illumination system the secondary illumination system is aligned with eye.

Example 142. The medical device of Example 141, wherein the first optical axis directed at the eye of the illumination system and the second optical axis directed at the eye of the secondary illumination system are angled relative to each other. Optionally, the first optical axis directed at the eye of the illumination system is angled at about 10 degrees to the second optical axis directed at the eye of the secondary illumination system.

Example 143. The medical device of Example 142, wherein in the second rotational position of the illumination system and the secondary illumination system the illumination system is outside of a field of view of the first camera and the optics of the secondary illumination system are within the field of view of the first camera.

Example 144. A medical device for imaging at least a portion of an eye of a patient is provided. The medical device comprising a first illumination system including a first illumination source and a second illumination system including a second illumination source. The first illumination system producing a first output to illuminate the eye. The first illumination system including a first optical assembly to project the first output towards the eye. The second illumination system producing a second output to illuminate the eye. The medical device further comprising an observation system including a second optical assembly positioned to receive imaging rays produced by reflection of light from the eye of the patient due to the first output of the first illumination system when the first illumination system, the second illumination system, and the observation system are positioned in a first spatial configuration relative to the eye and to receive imaging rays produced by reflection of light from the eye of the patient due to the second output of the second illumination system when the first illumination system, the second illumination system, and the observation system are positioned in a second spatial configuration relative to the eye. The second spatial configuration being different than the first spatial configuration. Both the first illumination system and the second illumination system being rotatable relative to the observation system. The observation system further including a first camera configured to receive the imaging rays from the second adjustable optical assembly. The medical device further comprising a controller operatively coupled to the first illumination system, the second illumination system, and to the observation system. The controller configured to selectively illuminate the eye with the first illumination system in the first spatial configuration and to illuminate the eye with the second illumination system in the second spatial configuration.

Example 145. The medical device of Example 144, wherein a first optical axis directed at the eye of the first illumination system and a second optical axis directed at the eye of the second illumination system are fixed relative to each other.

Example 146. The medical device of Example 145, wherein the first optical axis directed at the eye of the first illumination system and the second optical axis directed at the eye of the second illumination system are angled relative to each other. Optionally the first optical axis directed at the eye of the first illumination system is angled at about 10 degrees to the second optical axis directed at the eye of the second illumination system.

Example 147. The medical device of Example 146, wherein the first illumination system and the second illumination system rotate together relative to the eye.

Example 148. The medical device of any one of Examples 144-147, wherein in the second spatial configuration the first illumination system is outside of a field of view of the first camera of the observation system and a portion of the second illumination system is within the field of view of the first camera.

Example 149. The medical device of any one of Examples 144-147, wherein the first illumination system includes a first adjustable optical assembly and the observation system includes a second adjustable optical assembly. The controller being configured to adjust the first adjustable optical assembly of the first illumination system and the second adjustable optical system of the observation system to a plurality of focal depths relative to the eye while the first illumination system, the second illumination system, and observation system are in the first spatial configuration.

Example 150. The medical device of any one of Examples 144-149, wherein the second illumination source of the second illumination system is one of a laser diode, a fiber coupled diode laser, and a light emitting diode.

Example 151. The medical device of any one of Examples 144-150, wherein the second illumination source of the second illumination system emits a white illumination, a red illumination, or a near infrared illumination.

Example 152. A medical device for imaging at least a portion of an eye of a patient is provided. The medical device comprising an observation system including a first camera and an illumination system including a first illumination source. The observation system imaging the eye along a first axis. The first axis intersecting the eye at a first location. The first illumination system producing a first output to illuminate the eye and a first optical assembly to project the first output towards the eye. The first output projected to intersect the eye at a second location spaced apart from the first location. A portion of the first illumination system is within a field of view of the first camera of the observation system.

Example 153. The medical device of Example 152, wherein the portion is a first portion of the first optical assembly of the illumination system.

Example 154. The medical device of any one of Examples 152 and 153, wherein the first optical assembly expands an output of the first illumination source and focuses the expanded output at the second location.

Example 155. The medical device of any one of Examples 152-154, wherein the first location corresponds to an apex of a cornea of the eye and the second location corresponds to a portion of the cornea of the eye offset from the first axis.

Example 156. The medical device of any one of Examples 152-155, wherein the first output forms an illuminated area on a retina of the eye, the illuminated area including the first axis.

Example 157. The medical device of any one of Examples 152-156, wherein the second location is offset from the first location by about 1 millimeter.

Example 158. The medical device of any one of Examples 152-157, wherein the first output is angled relative to the first axis.

Example 159. The medical device of any one of Examples 152-158, wherein the first output is angled relative to the first axis by about 1 to about 3 degrees.

Example 160. The medical device of any one of Examples 152-159, wherein the first illumination source is one of a laser diode, a fiber coupled diode laser, and a light emitting diode.

Example 161. The medical device of any one of Examples 152-160, wherein the first illumination source emits a white illumination, a red illumination, or a near infrared illumination.

Example 162. A medical device for imaging at least a portion of an eye of a patient is provided. The medical device comprising an observation system including a first camera and an illumination system including a first illumination source. The observation system imaging the eye along a first axis. The first axis intersecting the eye at a first location corresponding to an apex of a cornea of the eye. The first illumination system producing a first output to illuminate the eye and a first optical assembly to project the first output towards the eye. The first output projected to intersect the eye at a second location spaced apart from the first location. The first output being focused at the second location to form an illuminated area on a retina of the eye. The illuminated area including the first axis.

Example 163. The medical device of Example 162, wherein the second location is offset from the first location by about 1 millimeter.

Example 164. The medical device of any one of Examples 162-163, wherein the first output is angled relative to the first axis.

Example 165. The medical device of any one of Examples 162-164, wherein the first output is angled relative to the first axis by about 1 to about 3 degrees.

Example 166. The medical device of any one of Examples 162-165, wherein the second illumination source of the second illumination system is one of a laser diode, a fiber coupled diode laser, and a light emitting diode.

Example 167. The medical device of any one of Examples 162-166, wherein the second illumination source of the second illumination system emits a white illumination, a red illumination, or a near infrared illumination.

Various modifications and additions can be made to the exemplary embodiments. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations of the disclosed embodiments together with all equivalents thereof.

1. A medical device for determining a separation from an eye, the medical device comprising:
an optical system positioned to receive reflected light from the eye, the optical system having an optical axis;
an image sensor positioned to detect an image formed by the optical system, the image sensor tilted relative to the optical axis of the optical system such that a first portion of the image sensor on a first side of a plane passing through the optical axis is closer to a rear face of the optical system than a second portion of the image sensor on a second side of the plane passing through the optical axis, the second side of the plane being opposite the first side of the plane; and
a controller configured to receive image data of the detected image from the image sensor and to determine the separation from the eye based on analyzing multiple portions of image data including at least one of a first portion of the image data from the first portion of the image sensor and a second portion of the image data from the second portion of the image sensor.

2. The medical device of claim 1, wherein the controller further analyzes at least a third portion of the image data, the third portion of image data being closer to the plane than the at least one of the first portion of the image data from the first portion of the image sensor and the second portion of the image data from the second portion of the image sensor.

3. The medical device of claim 2, wherein the controller analyzes both the first portion of the image data from the first portion of the image sensor and the second portion of the image data from the second portion of the image sensor.

4. The medical device of claim 1, wherein the controller analyzes both the first portion of the image data from the first portion of the image sensor and the second portion of the image data from the second portion of the image sensor.

5. The medical device of claim 1, wherein the controller analyzes a frequency component of the image data to determine the separation from the eye.

6. The medical device of claim 5, wherein the frequency component is a frequency domain component.

7. The medical device of claim 2, wherein the controller analyzes a frequency component of the image data to determine the separation from the eye.

8. The medical device of claim 7, wherein the frequency component is a frequency domain component.

9. The medical device of claim 3, wherein the controller analyzes a frequency component of the image data to determine the separation from the eye.

10. The medical device of claim 9, wherein the frequency component is a frequency domain component.

11. The medical device of claim 1, wherein the controller is configured to:
(a) convert the image data to a grayscale image data;
(b) determine a frequency domain of the grayscale image data;
(c) apply a first high pass filter to the frequency domain of the grayscale image to produce a high frequency domain image;
(d) apply a second high pass filter to the frequency domain of the grayscale image to produce a mid-to-high frequency domain image;
(e) determine a first spatial image of the high frequency domain image;
(f) determine a second spatial image of the mid-to-high frequency domain image;
(g) for each pixel of the image data determine a ratio of the pixel value in the first spatial image to the pixel value in the second spatial image to produce a focus map;
(h) determine a centroid of the focus map; and
(i) based on a position of the centroid determine the separation from the eye.

12. The medical device of claim 11, wherein the separation from the eye is determined by determining a pixel separation of the centroid from the plane.

13. The medical device of claim 12, wherein the pixel separation is converted to a distance.

14. The medical device of claim 1, further comprising:
an illumination system including a digital projector producing at least one test pattern image to illuminate the eye and a first adjustable optical assembly to project the at least one test pattern image towards the eye; and
an observation system including the optical system which is positioned to receive imaging rays produced by reflection of light from the eye of the patient due to the projection of the at least one test pattern image towards the eye by the illumination system and a first camera configured receive the imaging rays from the optical system the illumination system and the observation system being positioned in a first spatial configuration relative to the eye.

15. The medical device of claim 14, wherein the at least one test pattern includes a vertical slit and in a first image of the at least one test pattern image the vertical slit is in a first transverse position and in a second image of the at least one test pattern image the vertical slit is in a second transverse position, the second traverse position in the second image being spaced apart from the first transverse position in the first image.

16. The medical device of claim 15, wherein the controller focuses the first adjustable optical assembly of the illumination system at a first focal depth of a plurality of focal depths when the first image of the at least one test pattern image is produced by the digital projector to focus the vertical slit in the first transverse position on a cornea of the eye at a first location offset from an apex of the cornea of the eye and focuses the first adjustable optical assembly of the illumination system at a second focal depth of the plurality of focal depths when the second image of the at least one test pattern image is produced by the digital projector to focus the vertical slit in the second transverse position on the cornea at a second location offset from the apex of the cornea, the second location on the cornea being offset from the first location on the cornea.

17. The medical device of claim 16, wherein the controller further focuses the optical system of the observation system at the first focal depth of the plurality of focal depths when the first image of the at least one test pattern image is produced by the digital projector to focus the vertical slit in the first transverse position on the cornea at the first location offset from the apex of the cornea and focuses the optical system of the observation system at the second focal depth of the plurality of focal depths when the second image of the at least one image is produced by the digital projector to focus the vertical slit in the second transverse position on the cornea at the second location offset from the apex of the cornea.

18. The medical device of claim 16, wherein the controller is configured to adjust the first adjustable optical assembly of the illumination system and the optical system of the observation system to a plurality of focal depths relative to an apex of a cornea of the eye while the illumination system and observation system are in the first spatial configuration, wherein a first subset of the plurality of focal depths are part of a first contour and a second subset of the plurality of focal depths are part of a second contour, the second contour being offset relative to the first contour, and wherein the digital projector produces the first test pattern image having the vertical slit in the first transverse position for a first focal depth of the first subset of the plurality of focal depths of the first contour and a first focal depth of the second subset of the plurality of focal depths of the second contour and the digital projector produces the second test pattern image having the vertical slit in the second transverse position for a second focal depth of the first subset of the plurality of focal depths and a second focal depth of second subset of the plurality of focal depths.

19. The medical device of claim 18, wherein the first contour has a first shape and the second contour has a second shape, the second shape being different than the first shape.

20. The medical device of claim 19, wherein the second shape is flatter than the first shape.

21. The medical device of claim 14, further comprising a movable base, the illumination system being supported by the movable base and the observation system being supported by the movable base, the illumination system being movable relative to the movable base, the illumination system is rotatable relative to the observation system about a first rotation axis.

22. The medical device of claim 21, wherein the controller is further configured to determine a movement of the eye during an examination and to alter a position of the movable base based on the movement of the eye.

23. The medical device of claim 21, further comprising a patient support adapted to position the eye of the patient, wherein the movable base is moveable relative to the patient support.
